(19) United States
(12) Patent Application Publication
(10) Pub. No.: US 2018/0298447 A1
(43) Pub. Date: Oct. 18, 2018

(54) METHODS FOR ASSESSING THE TREATMENT RESPONSE OF CANCER PATIENTS AND FOR TREATING CANCER PATIENTS BY ANALYSING CPG METHYLATION

(71) Applicants: RHEINISCH FRIEDRICH-WILHELMS-UNIVERSITÄT BONN INSTITUT FÜR REKONSTRUKTIVE NEUROBIOLOGIE, Bonn (DE); MARTIN-LUTHER-UNIVERSITÄT HALLE-WITTENBERG, Halle (DE)

(72) Inventors: Michael FLEISCHHACKER, Berlin (DE); Bernd SCHMIDT, Berlin (DE); Dimo DIETRICH, Berlin (DE)

(21) Appl. No.: 15/524,619

(22) PCT Filed: Nov. 6, 2015
(86) PCT No.: PCT/EP2015/075870

§ 371 Date: May 4, 2017

Related U.S. Application Data
(60) Provisional application No. 62/076,674, filed on Nov. 7, 2014.

(51) Int. Cl. C12Q 1/6886

(52) U.S. Cl. CPC C12Q 1/6886; C12Q 2600/154; C12Q 2600/106

(57) ABSTRACT

The present invention relates to the field of pharmacogenomics and in particular to assessing the response of a cancer patient to a treatment by analysing CpG methylation in the shox2 gene. Depending on the result of the analysis, the treatment can be continued or altered, thereby exploiting the therapeutic window better than conventional methods of assessing a treatment response.

### FIELD OF THE INVENTION

The present invention relates to the field of pharmacogenomics and in particular to assessing the response of a cancer patient to a treatment by analysing CpG methylation in the shox2 gene. Depending on the result of the analysis, the treatment can be continued or changed, thereby exploiting the therapeutic window better than conventional methods of assessing a treatment response.

### BACKGROUND OF THE INVENTION

Lung cancer is still a huge health problem world-wide. In the US alone there will be approximately 224,210 new lung cancer cases in 2014. Lung cancer is expected to account for 13% of all new cancer diagnoses and 27% of all cancer related deaths in the US in 2014. The five-year relative survival rates for lung cancer for all stages is 16% and only slightly better than it was 30 years ago (Siegel R, Naishadham D, Jemal A. (2013) Cancer statistics, 2013. CA Cancer J Clin 63: 11-30). This poor outcome is mainly caused by the fact that the majority of patients (61%) have distant metastases at the time of diagnosis and palliative treatment remains the only option. In recent years several new therapy regimens were introduced, including a variety of different multimodal treatments in patients with locally advanced, late stage and metastatic disease (Reck M, Heigener D F, Mok T, Soria J C, Rabe K F. (2013) Management of non-small cell lung cancer: recent developments. Lancet 382: 709-719). These include the recent introduction of targeted therapies for molecularly selected patient subgroups like patients with an EGFR activation mutation, which has been made a standard for these patients. Additionally, several combination chemotherapies were introduced (Soria J C, Mauguen A, Reck M, Sandler A B, Saijo N, et al. (2013) Systematic review and meta-analysis of randomised, phase II/III trials adding bevacizumab to platinum based chemotherapy as first-line treatment in patients with advanced non-small-cell lung cancer. Ann Oncol 24: 20-30) and the maintenance regimens for patients with advanced stage non-small-cell lung carcinoma (NSCLC) have shown beneficial effects. There is also a survival advantage for patients which are treated with second-line chemotherapy as compared to best supportive care alone, and clinical trials testing new combinations in the second line setting for refractory disease were initiated (Fathi A T, Brahmer J R. (2008) Chemotherapy for advanced stage non-small cell lung cancer. Semin Thorac Cardiovasc Surg 20: 210-216). In addition, there is an increase in the number of active and more tolerable agents for treating NSCLC patients following induction therapy with a continuation maintenance or switch maintenance regimen (Varughese S, Jahangir K S, Simpson C E, Boulmay B C. (2012) A paradigm shift in the treatment of advanced non-small cell lung cancer. Am J Med Sci 344: 147-150). Since the therapeutic window is nevertheless rather small, it is of utmost importance to choose the best care for the patients, which includes the therapy regime with the highest probability of response and the search for strategies for an early and continuous assessment of treatment response. Advances in the systemic therapies not only lead to an improved survival but also to a reduction of cancer-related symptoms and a higher quality of life (Scheff R J, Schneider B J. (2013) Non-Small-Cell Lung Cancer: Treatment of Late Stage Disease: Chemotherapeutics and New Frontiers. Semin Intervent Radiol 30: 191-198).

Current standard of care consists of re-staging after two to four cycles of systemic therapy (i.e. after 6 to 12 weeks) using appropriate imaging techniques (CT, MRI, PET). The standard procedure for advanced stage lung cancer patients after induction of therapy is a CT scan to evaluate the tumor response (Goeckenjan G, Sitter H, Thomas M, Branscheid D, Flentje M, et al. (2010) Prevention, diagnosis, therapy, and follow-up of lung cancer. Pneumologie 64 Suppl 2: e1-164). Apart from the relatively high cost for a CT, the sensitivity of this imaging technique is rather low. The use of an automatic volumetry software tool makes it possible to detect an increase in tumor size of 26%, but whether this sensitivity is sufficient for judging a therapy response has to be demonstrated in the future. In addition, the inter-observer variability in the measurement of the size is prone to misinterpretation of tumor response (Erasmus J J, Gladish G W, Broemeling L, Sabloff B S, Truong M T, et al. (2003) Interobserver and intraobserver variability in measurement of non-small-cell carcinoma lung lesions: implications for assessment of tumor response. J Clin Oncol 21: 2574-2582).

So far there are a few potentially useful biomarkers that sometimes correlate with response to a given therapy (although to the inventors' knowledge there are no clear clinical data demonstrating their usefulness); like CYFRA-21, SCCA, CEA and CA-125 for NSCLC patients and ProGRP and NSE for small-cell lung carcinoma (SCLC) patients (reviewed in Cho W C. (2007) Potentially useful biomarkers for the diagnosis, treatment and prognosis of lung cancer. Biomed Pharmacother 61: 515-519). Apart from the fact that there is no universal marker useful for all different lung cancer histologies, the published data still remain controversial and so far there is not enough evidence for any of them to be routinely used in the clinic. Amongst the biomarkers that have been tested for clinical usefulness to predict a response to chemotherapy, but with limited success, were eight immunohistochemical biomarkers, none of which could predict chemotherapy response and survival rate (Toffart A C, Timsit J F, Couraud S, Merle P, Moro-Sibilot D, et al. (2013) Immunohistochemistry evaluation of biomarker expression in non-small cell lung cancer (Pharmacogeno scan study). Lung Cancer) nor prove a strong correlation between marker level and treatment response. When neuron specific enolase (NSE) was used for monitoring SCLC patients, this marker was found to be only useful in patients with an increased pre-treatment level (Splinter T A, Carney D N, Teeling M, Peake M D, Kho G S, et al. (1989) Neuron specific enolase can be used as the sole guide to treat small-cell lung cancer patients in common clinical practice. J Cancer Res Clin Oncol 115: 400-401). A similar observation was published by Johnson et al who found that NSE, lactate dehydrogenase (LDH) and chromogranin A did not correlate with treatment response (Johnson P W, 315 Joel S P, Love S, Butcher M, Pandian M R, et al. (1993) Tumour markers for prediction of survival and monitoring of remission in small cell lung cancer. Br J Cancer 67: 760-766). A longitudinal measurement of serum soluble interleukin 2 receptor demonstrated a reduction in serum concentration under therapy, but this was not a sign for disease remission (Brunetti G, Bossi A, Baiardi P, Jedrychowska I, Pozzi U, et al. (1999) Soluble interleukin 2 receptor (sIL2R) in monitoring advanced lung cancer during chemotherapy. Lung Cancer 23: 1-9). Thymidine kinase (TK) was unable to discriminate between the various response groups of lung cancer patients (Holdenrieder S, von P J, Duell T, Feldmann K, Raith H, et al. (2010) Clinical relevance of thymidine kinase for the diagnosis, therapy monitoring and prognosis of non-operable lung cancer. Anticancer Res 30: 1855-1862). In contrast to these reports there are a few biomarkers for therapy monitoring in lung cancer patients, like CYFRA 21-1 and nucleosome levels, but none of them is routinely used in the clinic (Holdenrieder S, Stieber P, von P J, Raith H, Nagel D, et al. (2004) Circulating nucleosomes predict the response to chemotherapy in patients with advanced non-small cell lung cancer. Clin Cancer Res 10: 5981-5987; Holdenrieder S, von P J, Dankelmann E, Duell T, Faderl B, et al. (2009) Nucleosomes and CYFRA 21-1 indicate tumor response after one cycle of chemotherapy in recurrent non-small cell lung cancer. Lung Cancer 63: 128-135; Holdenrieder S, von P J, Dankelmann E, Duell T, Faderl B, et al. (2008) Nucleosomes, ProGRP, NSE, CYFRA 21-1, and CEA in monitoring first-line chemotherapy of small cell lung cancer. Clin Cancer Res 14: 7813-7821).

In order to optimize the selection of treatment options, a rapid, specific and sensitive method for the assessment of a therapy response is of crucial importance.

More than 65 years ago Mandel and Metais described for the first time their observation of the presence of extracellular nucleic acids in humans (Mandel P, Metais P. Les acides nucleiques du plasma sanguin chez l'homme. C. R. Acad. Sci. Paris 142, 241-243. 1948) and more than four decades later it could be clearly demonstrated that tumor-associated genetic alterations can be found in cell-free nucleic acids isolated from plasma, serum and other body fluids (Fleischhacker M, Schmidt B. (2007) Circulating nucleic acids (CNAs) and cancer—a survey. Biochim Biophys Acta 1775: 181-232; Jung K, Fleischhacker M, Rabien A. (2010) Cell-free DNA in the blood as a solid tumor biomarker-a critical appraisal of the literature. Clin Chim Acta 411: 1611-1624). According to our current knowledge it seems as if some tumor-associated alterations found in tumor cells can also be detected in extracellular nucleic acids. This includes epigenetic alterations observed in different forms of malignant tumors. A hallmark of mammalian chromatin is DNA methylation and it is known that cytosine methylation in the context of a CpG dinucleotide plays a role in the regulation of development and is important in basic biological processes like embryogenesis and cell differentiation (Smith Z D, Meissner A. (2013) DNA methylation: roles in mammalian development. Nat Rev Genet 14: 204-220; Gibney E R, Nolan C M. (2010) Epigenetics and gene expression. Heredity (Edinb) 105: 4-13). As such, methylation not only regulates gene transcription, but also plays a role in maintaining genome stability, imprinting and X-chromosome inactivation. Epigenetic alterations in oncogenes and tumor suppressor genes are of key importance in the development of cancer (Suva M L, Riggi N, Bernstein B E. (2013) Epigenetic reprogramming in cancer. Science 339: 1567-1570).

An assay for the quantitative determination of extracellular methylated SHOX2 DNA (mSHOX2) is known for the discrimination of patients with a benign lung disease from patients with lung cancer (Schmidt B, Liebenberg V, Dietrich D, Schlegel T, Kneip C, et al. (2010) SHOX2 DNA methylation is a biomarker for the diagnosis of lung cancer based on bronchial aspirates. BMC Cancer 10: 600; Kneip C, Schmidt B, Seegebarth A, Weickmann S, Fleischhacker M, et al. (2011) SHOX2 DNA methylation is a biomarker for the diagnosis of lung cancer in plasma. J Thorac Oncol 6: 1632-1638). Recently, it had been shown that the quantification of mSHOX2 is also a useful tool for improving the accuracy in lung cancer staging by endobronchial ultrasound with transbronchial needle aspiration (Darwiche K, Zarogoulidis P, Baehner K, Welter S, Tetzner R, et al. (2013) Assessment of SHOX2 methylation in EBUS-TBNA specimen improves accuracy in lung cancer staging. Ann Oncol).

The present inventors unexpectedly found that a quantitative determination of mSHOX2 is useful for the determination of a treatment response for advanced stage lung cancer patients. The principle is to use free circulating tumor DNA from the lung cancer marker mSHOX2 that is released from the tumor into the blood as an indicator for lung tumor load in the body of the patient. In patients that have measurable mSHOX2 levels in the blood pre-treatment, mSHOX2 levels determined in the blood after start of the treatment can be used to monitor the treatment response. In responders, a substantial decrease in mSHOX2 levels in the blood can be observed reflecting tumor shrinkage—even before this is detected with other methods. One reason for the fast decrease of the plasma mSHOX2 values in patients responding to the therapy is the short half-life of extracellular nucleic acids, which was determined to be less than six hours in an animal model (Rago C, Huso D L, Diehl F, Karim B, Liu G, et al. (2007) Serial assessment of human tumor burdens in mice by the analysis of circulating DNA. Cancer Res 67: 9364-9370). There are additional advantages for a treatment monitoring using mSHOX2 quantification. The method is useful even for patients with a very low pre-therapeutic mSHOX2 value of percent of methylated reference (PMR) ≥1% (whereby PMR is the amount of methylated marker DNA in relation to a control reference measured in parallel representing the total DNA in percent). For patients that are eligible for monitoring, a single measurement at a defined time point after the start of a therapy is able to determine a response earlier and more reliable than other methods. The earlier assessment is thought to be possible because the mSHOX2 quantification is surprisingly far more sensitive regarding tumor size change than imaging techniques. Also the method is equally well suited for the monitoring of NSCLC and SCLC patients alike.

The ability to isolate and to characterize extracellular nucleic acids from tumor patients with very sensitive and highly specific methods led to the concept of “liquid biopsy”. Therefore, follow-up analysis of tumor patients is possible by longitudinally analyzing the extracellular nucleic acids to follow the reaction of a tumor to a given therapy, or the development of resistance mechanisms. As a result, physicians no longer depend exclusively on a single examination of tissue biopsies (usually at the time of diagnosis) and CT scans done for re-staging the patients. Also, the treatment response can be assessed far earlier than by conventional methods. This allows for improved care for cancer patients, since the treatment can be adapted to the patient response far earlier than before, providing for a much better use of the small therapeutic window.

### SUMMARY OF THE INVENTION

In a first aspect, the present invention relates to a method for monitoring a cancer under treatment, comprising determining the amount of hypermethylated shox2 genomic DNA in a first and one or more further test samples of a cancer patient taken subsequently.

In a second aspect, the present invention relates to a method for predicting the effect of a cancer treatment, comprising determining the amount of hypermethylated shox2 genomic DNA in a first and one or more further test samples of a cancer patient taken subsequently, wherein the change in the amount of hypermethylated shox2 genomic DNA indicates the effect of the treatment.

In a third aspect, the present invention relates to a method for identifying a patient as a responder to a cancer treatment, comprising determining the amount of hypermethylated shox2 genomic DNA in a first and one or more further test samples of a cancer patient taken subsequently, wherein a substantial decrease in the amount of hypermethylated shox2 genomic DNA indicates a response to the treatment.

In a fourth aspect, the present invention relates to a method for identifying a patient as a non-responder to a cancer treatment, comprising determining the amount of hypermethylated shox2 genomic DNA in a first and one or more further test samples of a cancer patient taken subsequently, wherein an increase, a stagnation or a non-substantial decrease in the amount of hypermethylated shox2 genomic DNA indicates a non-response to the treatment.

In a fifth aspect, the present invention relates to a method for treating cancer, comprising the steps:
(i) determining the amount of hypermethylated shox2 genomic DNA in a first test sample of a cancer patient;
(ii) starting treatment of said patient with a first treatment regimen comprising one or more anti-cancer agents or therapies,
(iii) determining the amount of hypermethylated shox2 genomic DNA in one or more subsequently taken further test samples of said patient;
(iv) optionally repeating steps (ii) and (iii) one or more times;
(v) continuing treating the patient with the first treatment regimen if there is a substantial decrease in the amount of hypermethylated shox2 genomic DNA, or terminating treating the patient with the first treatment regimen and treating the patient instead with a second treatment regimen comprising one or more anti-cancer agents or therapies not comprised in the first treatment regimen if there is an increase, a stagnation or a non-substantial decrease in the amount of hypermethylated shox2 genomic DNA.

### LEGENDS TO THE FIGURES

FIG. 1: Trend curves of mSHOX2 percent of methylated reference (PMR) with linear scale axis (top plot A) and logarithmic scale (bottom plot B) for patients responding and not responding to the therapy. Patients who do not respond to therapy are shown in gray and patients who do respond to therapy are shown in black. The patients included in this figure are limited to the ones with a baseline mSHOX2 PMR of at least 1% PMR. The first blood draw (x=0) is the point of diagnosis, i.e. before treatment and defines the baseline methylation of shox2 for each patient. The logarithmic scale on the bottom is based on a started log (log10 of PMR +0.1%) to circumvent the impossibility of log (0). For the first eight blood draws Bonferroni corrected p-values from unpaired two sample Wilcox tests are given at the bottom.

FIG. 2: Paired Boxplots of mSHOX2 PMRs for patients responding and not responding to the therapy (corresponding to data in FIG. 1 top) at 9 time points of blood draws: at base line and at time point 1-8. Only patients with a baseline PMR ≥1% were included.

FIG. 3: ROC curves for the discrimination of responders from non-responder at different blood draws. Only patients with a baseline PMR ≥1% were included. The classification of the patients in either group is based on observation of the tumors by CT scans as the gold standard. This was performed by the local tumor board and completely independent from the mSHOX2 measurements. The first blood draw (time 0) is the point before treatment (=baseline methylation). Blood draws 1 to 8 were done during the therapy of the patients at intervals of 7 to 10 days.

FIG. 4: Regions of interest (ROIs), bisulfite sequenced regions (see Example 2) and assays (see Example 1) in SHOX2 (respective SEQ ID NOs in brackets, compare Table 3).

FIG. 5: DNA methylation of CpGs in 6 amplificates assessed for SHOX2 using Direct Bisulfite Sequencing (11 lung cancer patients and 12 healthy patients). Each column is a tissue from an individual that either was a lung cancer patient (group on the left side) or healthy (group on the right side). Each row is a CpG, whereby CpGs are grouped by amplificate and ordered by their position in the amplificates with the Ids as given on the left. The Amplificates are ordered by their position in SHOX2 (compare FIG. 4). The CpG methylation is coded in a greyscale from light grey (0%) to dark grey (100%)—completely white parts lack measurements for CpGs. P-values from Wilcox tests for cancer vs. healthy are given on the right for each individual CpG where such test was possible.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

The specification refers to thirty six SEQ IDs. An overview and explanation of these SED IDs is given in the following Table 1:

TABLE 1
SEQ IDs of the specification. rev.comp. means
reverse complement, converted (C−>T) or simply
(C−>T) means bisulfite conversion of cytosines outside
of CpG context into uracile and replaced by thymidine in
subsequent amplification. Subregions of shox2 are further described in
Table 3. Conversion of C−>T is only for cytosines in a non-CpG context.

| Seq ID | derived from | Type |
|---|---|---|
| 1 | | genomic shox2 |
| 2 | 1 | converted strand (C−>T) |
| 3 | 1 | (C−>T) and rev.comp. |
| 4 | 1 | rev. comp. and (C−>T) |
| 5 | 1 | rev. comp. (C−>T) rev.comp. |
| 6 | | genomic (shox2 subregion) |
| 7 | 6 | (C−>T) |
| 8 | 6 | (C−>T) and rev.comp. |
| 9 | 6 | rev. comp. and (C−>T) |
| 10 | 6 | rev. comp. (C−>T) rev.comp. |
| 11 | | genomic (shox2 subregion) |
| 12 | 11 | (C−>T) |
| 13 | 11 | (C−>T) and rev.comp. |
| 14 | 11 | rev. comp. and (C−>T) |
| 15 | 11 | rev. comp. (C−>T) rev.comp. |
| 16 | | genomic (shox2 subregion) |
| 17 | 16 | (C−>T) |
| 18 | 16 | (C−>T) and rev.comp. |
| 19 | 16 | rev. comp. and (C−>T) |
| 20 | 16 | rev. comp. (C−>T) rev.comp. |
| 21 | | genomic (shox2 subregion) |
| 22 | 21 | (C−>T) |
| 23 | 21 | (C−>T) and rev.comp. |
| 24 | 21 | rev. comp. and (C−>T) |
| 25 | 21 | rev. comp. (C−>T) rev.comp. |
| 26 | | genomic (shox2 subregion) |
| 27 | 26 | (C−>T) |
| 28 | 26 | (C−>T) and rev.comp. |
| 29 | 26 | rev. comp. and (C−>T) |
| 30 | 26 | rev. comp. (C−>T) rev.comp. |
| 31 | | shox2 forward primer |
| 32 | | shox2 forward blocker |
| 33 | | shox2 reverse primer |
| 34 | | shox2 probe |
| 35 | | shox2 reverse primer B |
| 36 | | shox2 reverse blocker |

The authors of the present invention found that the response to the treatment of cancer can be assessed much earlier than conventionally possible by analyzing the methylation status of the shox2 gene. According to these findings, in a first aspect, the present invention relates to a method for monitoring a cancer under treatment, comprising determining the amount of methylated shox2 genomic DNA in a first and one or more further test samples of a cancer patient taken subsequently.

The term “monitoring” as used herein refers to the accompaniment of a diagnosed cancer during a treatment procedure or during a certain period of time, typically during at least 1 week, 2 weeks, 3 weeks, 4 weeks, 1 month, 2 months, 3 months, 4 months, 5 months, 6 months, 1 year, 2 years, 3 years, 5 years, 10 years, or any other period of time. The term “accompaniment” means that states of and, in particular, changes of these states of a cancer may be detected based on the amount of hypermethylated shox2 genomic DNA, particular based on changes in the amount in any type of periodical time segment, determined e.g., daily or 1, 2, 3, 4, 5, 6, 7, 8, 9, 10, 11, 12, 13, 14 or 15 times per month (no more than one determination per day) over the course of the treatment, which may be up to 1, 2, 3, 4, 5, 6, 7, 8, 9, 10, 11, 12, 15 or 24 months. Amounts or changes in the amounts can also be determined at treatment specific events, e.g. before and/or after every treatment cycle or drug/therapy administration.

A cycle is the time between one round of treatment until the start of the next round. Cancer treatment is usually not a single treatment, but a course of treatments. A course usually takes between 3 to 6 months, but can be more or less than that. During a course of treatment, there are usually between 4 to 8 cycles of treatment. Usually a cycle of treatment includes a treatment break to allow the body to recover.

The term “cancer” as used herein refers to a large family of diseases which involve abnormal cell growth with the potential to invade or spread to other parts of the body. The cells form a subset of neoplasms or tumors. A neoplasm or tumor is a group of cells that have undergone unregulated growth, and will often form a mass or lump, but may be distributed diffusely. Preferably, the term “cancer” is defined by one or more of the following characteristics:

self-sufficiency in growth signalling,
insensitivity to anti-growth signals,
evasion of apoptosis,
enabling of a limitless replicative potential,
induction and sustainment of angiogenesis, and/or
activation of metastasis and invasion of tissue.

The cancer may be selected from the group consisting of Adrenal Cancer, Anal Cancer, Bile Duct Cancer, Bladder Cancer, Bone Cancer, Brain/CNS Tumors, Breast Cancer, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Liver Cancer, Lung Cancer, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Non-Hodgkin Lymphoma, Oral Cavity and Oropharyngeal Cancer, Osteo sarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor. In a preferred embodiment, the cancer is cancer comprising cancer cells in which the gene shox2 is hypermethylated. In a more preferred embodiment, the cancer is lung cancer. The lung cancer may be small cell lung cancer (SCLC) or non-small cell lung cancer (NSCLC).

The term “SCLC” or “small cell lung cancer”, as used herein, refers to an undifferentiated neoplasm, preferably composed of primitive- or embryonic-appearing cells. As the name implies, the cells in small-cell carcinomas are smaller than normal cells and barely have room for any cytoplasm.

The term “NSCLC” or “non-small cell lung cancer”, as used herein, refers to a group of heterogeneous diseases grouped together because their prognosis and management is roughly identical and includes, according to the histological classification of the World Health Organization/International Association for the Study of Lung Cancer (Travis W D et al. Histological typing of lung and pleural tumors. 3^{rd} ed. Berlin: Springer-Verlag, 1999):
(i) Squamous cell carcinoma (SCC), accounting for 30% to 40% of NSCLC, starts in the larger breathing tubes but grows slower meaning that the size of these tumors varies on diagnosis.
(ii) Adenocarcinoma is the most common subtype of NSCLC, accounting for 50% to 60% of NSCLC, which starts near the gas-exchanging surface of the lung and which includes a subtype, the bronchioalveolar carcinoma, which may have different responses to treatment.
(iii) Large cell carcinoma is a fast-growing form that grows near the surface of the lung. It is primarily a diagnosis of exclusion, and when more investigation is done, it is usually reclassified to squamous cell carcinoma or adenocarcinoma.
(iv) Adenosquamous carcinoma is a type of cancer that contains two types of cells: squamous cells (thin, flat cells that line certain organs) and gland-like cells.
(v) Carcinomas with pleomorphic, sarcomatoid or sarcomatous elements. This is a group of rare tumors reflecting a continuum in histological heterogeneity as well as epithelial and mesenchymal differentiation.
(vi) Carcinoid tumor is a slow-growing neuroendocrine lung tumor and begins in cells that are capable of releasing a hormone in response to a stimulus provided by the nervous system.
(vii) Carcinomas of salivary gland type begin in salivary gland cells located inside the large airways of the lung.
(viii) Unclassified carcinomas include cancers that do not fit into any of the aforementioned lung cancer categories.

In one embodiment, the NSCLC is a squamous cell carcinoma, adenocarcinoma, large cell (undifferentiated) carcinoma, adenosquamous carcinoma and sarcomatoid carcinoma. The NSCLC may be a stage 0, IA, IB, IIa, IIb, Ma, Mb or IV NSCLC.

The term “stage I NSCLC”, as used herein, refers to tumor which is present in the lungs but the cancer has not been found in the chest lymph nodes or in other locations outside of the chest. Stage I NSCLC is subdivided into stages IA and IB, usually based upon the size of the tumor or involvement of the pleura, which is lining along the outside of the lung. In Stage IA, the tumor is 3 centimeters (cm) or less in size and has invaded nearby tissue minimally, if at all. The cancer has not spread to the lymph nodes or to any distant sites. In Stage IB, the tumor is more than 3 cm in size, has invaded the pleural lining around the lung, or has caused a portion of the lung to collapse. The cancer has not spread to the lymph nodes or to any distant sites. Stage IA corresponds to stages T1N0M9 of the TNM classification. Stage IB corresponds to T2M0N0 of the TNM classification.

The term “Stage II NSCLC”, as used herein, refers to a cancer which has either begun to involve the lymph nodes within the chest or has invaded chest structures and tissue more extensively. However, no spread can be found beyond the involved side of the chest, and the cancer is still considered a local disease. Stage II is subdivided into stages IIA and IIB. Stage IIA refers to tumors which are 3 cm or smaller and have invaded nearby tissue minimally, if at all. One or more lymph nodes on the same side of the chest are involved, but there is no spread to distant sites. Stage IIB is assigned in two situations: when there is a tumor larger than 3 cm with some invasion of nearby tissue and involvement of one or more lymph nodes on the same side of the chest; or for cancers that have no lymph node involvement, but have either invaded chest structures outside the lung or are located within 2 cm of the carina (the point at which the trachea, or the tube that carries air to the lungs, splits to reach the right and left lungs). Stage IIA corresponds to T1N1M0 or T2N1M0 of the TNM classification. Stage IIB corresponds to T3N0M0 according to the TNM classification.

The term “Stage III NSCLC”, as used herein, refers to tumors which have invaded the tissues in the chest more extensively than in stage II, and/or the cancer has spread to lymph nodes in the mediastinum. However, spread (metastasis) to other parts of the body is not detectable. Stage III is divided into stages IIIA and IIIB. Stage IIIA refers to a single tumor or mass that is not invading any adjacent organs and involves one or more lymph nodes away from the tumor, but not outside the chest. Stage IIIB refers to a cancer which has spread to more than one area in the chest, but not outside the chest. Stage IIIA corresponds to T1N2M0, T2N2M0, T3N1M0, T3N2M0, T4N0M0 or T4N1M0 according to the TNM classification. Stage IIIB corresponds to T1N3M0, T2N3M0, T3N3M0, T4N2M0 or T4N3M0 according to the TNM classification.

The term “Stage IV NSCLC”, as used herein, refers to a cancer which has spread, or metastasized, to different sites in the body, which may include the liver, brain or other organs. Stage IV corresponds to any T or any N with M1.

The TNM classification is a staging system for malignant cancer. As used herein the term “TNM classification” refers to the 6^{th} edition of the TNM stage grouping as defined in Sobin et al. (International Union Against Cancer (UICC), TNM Classification of Malignant tumors, 6^{th} ed. New York; Springer, 2002, pp. 191-203) (TNM6) and AJCC Cancer Staging Manual 6th edition; Chapter 19; Lung—original pages 167-177 whereby the tumors are classified by several factors, namely, T for tumor, N for nodes, M for metastasis as follows:

T: Primary tumor cannot be assessed, or tumor proven by the presence of malignant cells in sputum or bronchial washings but not visualized by imaging or bronchoscopy:
T0: No evidence of primary tumor,
T is: Carcinoma in situ,
T1: Tumor 3 cm or less in greatest dimension, surrounded by lung or visceral pleura, without bronchoscopic evidence of invasion more proximal than the lobar bronchus (for example, not in the main bronchus),
T2: Tumor more than 3 cm but 7 cm or less or tumor with any of the following features (T2 tumors with these features are classified T2a if 5 cm or less): involves main bronchus, 2 cm or more distal to the carina; invades visceral pleura (PL1 or PL2); associated with atelectasis or obstructive pneumonitis that extends to the hilar region but does not involve the entire lung,
T3: Tumor more than 7 cm or one that directly invades any of the following: parietal pleural (PL3), chest wall (including superior sulcus tumors), diaphragm, phrenic nerve, mediastinal pleura, parietal pericardium; or tumor in the main bronchus less than 2 cm distal to the carina but without involvement of the carina; or associated atelectasis or obstructive pneumonitis of the entire lung or separate tumor nodule(s) in the same lobe and
T4: Tumor of any size that invades any of the following: mediastinum, heart, great vessels, trachea, recurrent laryngeal nerve, esophagus, vertebral body, carina, separate tumor nodule(s) in a different ipsilateral lobe.

### N (Regional Lymph Nodes):

NX: Regional lymph nodes cannot be assessed
N0: No regional lymph node metastases
N1: Metastasis in ipsilateral peribronchial and/or ipsilateral hilar lymph nodes and intrapulmonary nodes, including involvement by direct extension
N2: Metastasis in ipsilateral mediastinal and/or subcarinal lymph node(s)
N3: Metastasis in contralateral mediastinal, contralateral hilar, ipsilateral or contralateral scalene, or supraclavicular lymph node(s)
   M: Distant metastasis
M0: No distant metastasis
M1: Distant metastasis

In one embodiment, the lung cancer is an advanced stage lung cancer. An advanced SCLC or NSCLC is a cancer that has spread to other regions of the chest or other parts of the body. An advanced NSCLC is preferably a stage IIIb or IV NSCLC. In another embodiment, the advanced lung cancer shows lung or pleura metastasis.

The term “treatment” as used herein refers to a therapeutic treatment, wherein the goal is to reduce progression of cancer. Beneficial or desired clinical results include, but are not limited to, release of symptoms, reduction of the length of the disease, stabilized pathological state (specifically not deteriorated), slowing down of the disease's progression, improving the pathological state and/or remission (both partial and total), preferably detectable. A successful treatment does not necessarily mean cure, but it can also mean a prolonged survival, compared to the expected survival if the treatment is not applied. In a preferred embodiment, the treatment is a first line treatment, i.e. the cancer was not treated previously.

The term “determining the amount” as used herein refers to a quantification of hypermethylated shox2 genomic DNA and can be performed as described below. The quantification is preferably absolute, e.g. in pg per mL or ng per mL sample, copies per mL sample, number of PCR cycles etc., or relative, e.g. 10 fold higher than in a control sample or as percentage of methylation of a reference control.

The term “hypermethylated” as used herein relates to “methylation” or “DNA methylation”, which refers to a biochemical process involving the addition of a methyl group to the cytosine or adenine DNA nucleotides. DNA methylation at the 5 position of cytosine, especially in promoter regions, can have the effect of reducing gene expression and has been found in every vertebrate examined. In adult non-gamete cells, DNA methylation typically occurs in a CpG site. The term “CpG site” or “CpG dinucleotide”, as used herein, refers to regions of DNA where a cytosine nucleotide occurs next to a guanine nucleotide in the linear sequence of bases along its length. “CpG” is shorthand for “C-phosphate-G”, that is, cytosine and guanine separated by only one phosphate; phosphate links any two nucleosides together in DNA. The “CpG” notation is used to distinguish this linear sequence from the CG base-pairing of cytosine and guanine. Cytosines in CpG dinucleotides can be methylated to form 5-methylcytosine. The term “hypermethylation” refers to an aberrant methylation pattern or status (i.e. the presence or absence of methylation of one or more nucleotides), wherein one or more nucleotides, preferably C(s) of a CpG site(s), are methylated compared to the same genomic DNA from a non-cancer cell of the patient or a subject not suffering or having suffered from the cancer the patient is treated for, preferably any cancer (healthy control). In particular, it refers to an increased presence of 5-mCyt at one or a plurality of CpG dinucleotides within a DNA sequence of a test DNA sample, relative to the amount of 5-mCyt found at corresponding CpG dinucleotides within a healthy control DNA sample. Hypermethylation as a methylation status/pattern can be determined at one or more CpG site(s). If more than one CpG site is used, hypermethylation can be determined at each site separately or as an average of the CpG sites taken together. Alternatively, all assessed CpG sites must be methylated such that the requirement hypermethylation is fulfilled.

The term “shox2” or “SHOX2” as used herein refers to the shox2 (short stature homeobox 2, NCBI gene ID 6474, genomic location 3q25.32) gene, also designated homeobox protein Og12X or paired-related homeobox protein SHOT. It is a member of the homeobox family of genes that encode proteins containing a 60-amino acid residue motif that represents a DNA-binding domain. Homeobox proteins have been characterized extensively as transcriptional regulators involved in pattern formation in both invertebrate and vertebrate species. The genomic DNA sequence of human shox2 (chromosome 3 position 158095954 to 158106503 in GRCh38 genome build) is shown in SEQ ID NO: 1.

The term “mshox2” or “mSHOX2” as used herein refers to methylated shox2. It does not necessarily refer to fully methylated shox2, but to shox2 which is methylated at the CpG sites to be assessed. In one embodiment, it refers to hypermethylated shox2 as defined herein.

The CpG sites comprised in SEQ ID NO: 1 are as shown in Table 2.

TABLE 2
Position of CpGs in SHOX2 genomic DNA sequence (SEQ ID NO:
1) chromosome 3 position 158095954 to 158106503 in GRCh38
genome build (the position denotes the C of the CpG).

| N | Pos. |
|---|---|
| 1 | 14 |
| 2 | 49 |
| 3 | 68 |
| 4 | 89 |
| 5 | 149 |
| 6 | 172 |
| 7 | 281 |
| 8 | 380 |
| 9 | 584 |
| 10 | 655 |
| 11 | 761 |
| 12 | 782 |
| 13 | 787 |
| 14 | 1136 |
| 15 | 1239 |
| 16 | 1308 |
| 17 | 1322 |
| 18 | 1343 |
| 19 | 1351 |
| 20 | 1360 |
| 21 | 1403 |
| 22 | 1466 |
| 23 | 1514 |
| 24 | 1521 |
| 25 | 1658 |
| 26 | 1669 |
| 27 | 1733 |
| 28 | 1746 |
| 29 | 1761 |
| 30 | 1763 |
| 31 | 1811 |
| 32 | 1840 |
| 33 | 1852 |
| 34 | 1865 |
| 35 | 1874 |
| 36 | 1881 |
| 37 | 1900 |
| 38 | 1917 |
| 39 | 1930 |
| 40 | 1940 |
| 41 | 1959 |
| 42 | 1971 |
| 43 | 1978 |
| 44 | 1997 |
| 45 | 2008 |
| 46 | 2013 |
| 47 | 2015 |
| 48 | 2022 |
| 49 | 2038 |
| 50 | 2040 |
| 51 | 2049 |
| 52 | 2051 |
| 53 | 2066 |
| 54 | 2072 |
| 55 | 2091 |
| 56 | 2094 |
| 57 | 2121 |
| 58 | 2124 |
| 59 | 2154 |
| 60 | 2163 |
| 61 | 2167 |
| 62 | 2176 |
| 63 | 2181 |
| 64 | 2184 |
| 65 | 2191 |
| 66 | 2197 |
| 67 | 2199 |
| 68 | 2206 |
| 69 | 2211 |
| 70 | 2215 |
| 71 | 2223 |
| 72 | 2230 |
| 73 | 2254 |
| 74 | 2256 |
| 75 | 2260 |
| 76 | 2262 |
| 77 | 2272 |
| 78 | 2290 |
| 79 | 2292 |
| 80 | 2296 |
| 81 | 2304 |
| 82 | 2323 |
| 83 | 2347 |
| 84 | 2352 |
| 85 | 2402 |
| 86 | 2405 |
| 87 | 2447 |
| 88 | 2480 |
| 89 | 2551 |
| 90 | 2558 |
| 91 | 2581 |
| 92 | 2588 |
| 93 | 2616 |
| 94 | 2661 |
| 95 | 2727 |
| 96 | 2749 |
| 97 | 2814 |
| 98 | 2858 |
| 99 | 2880 |
| 100 | 2932 |
| 101 | 3094 |
| 102 | 3130 |
| 103 | 3238 |
| 104 | 3447 |
| 105 | 3741 |
| 106 | 3936 |
| 107 | 3951 |
| 108 | 3978 |
| 109 | 4033 |
| 110 | 4076 |
| 111 | 4342 |
| 112 | 4446 |
| 113 | 4785 |
| 114 | 5065 |
| 115 | 5540 |
| 116 | 5624 |
| 117 | 5699 |
| 118 | 5731 |
| 119 | 5751 |
| 120 | 5778 |
| 121 | 5847 |
| 122 | 6023 |
| 123 | 6104 |
| 124 | 6146 |
| 125 | 6158 |
| 126 | 6183 |
| 127 | 6187 |
| 128 | 6273 |
| 129 | 6372 |
| 130 | 6445 |
| 131 | 6646 |
| 132 | 6703 |
| 133 | 6732 |
| 134 | 6740 |
| 135 | 6753 |
| 136 | 6771 |
| 137 | 6781 |
| 138 | 6784 |
| 139 | 6799 |
| 140 | 6849 |
| 141 | 6855 |
| 142 | 6883 |
| 143 | 6899 |
| 144 | 6912 |
| 145 | 6926 |
| 146 | 6952 |
| 147 | 6962 |
| 148 | 6974 |
| 149 | 6990 |
| 150 | 7002 |
| 151 | 7006 |
| 152 | 7042 |
| 153 | 7072 |
| 154 | 7087 |
| 155 | 7104 |
| 156 | 7120 |
| 157 | 7128 |
| 158 | 7147 |
| 159 | 7151 |
| 160 | 7195 |
| 161 | 7202 |
| 162 | 7210 |
| 163 | 7236 |
| 164 | 7277 |
| 165 | 7292 |
| 166 | 7316 |
| 167 | 7321 |
| 168 | 7359 |
| 169 | 7361 |
| 170 | 7390 |
| 171 | 7413 |
| 172 | 7437 |
| 173 | 7455 |
| 174 | 7491 |
| 175 | 7504 |
| 176 | 7520 |
| 177 | 7531 |
| 178 | 7543 |
| 179 | 7549 |
| 180 | 7555 |
| 181 | 7562 |
| 182 | 7577 |
| 183 | 7622 |
| 184 | 7627 |
| 185 | 7635 |
| 186 | 7639 |
| 187 | 7645 |
| 188 | 7649 |
| 189 | 7659 |
| 190 | 7665 |
| 191 | 7690 |
| 192 | 7693 |
| 193 | 7702 |
| 194 | 7721 |
| 195 | 7736 |
| 196 | 7742 |
| 197 | 7746 |
| 198 | 7756 |
| 199 | 7776 |
| 200 | 7786 |
| 201 | 7794 |
| 202 | 7802 |
| 203 | 7812 |
| 204 | 7843 |
| 205 | 7852 |
| 206 | 7854 |
| 207 | 7861 |
| 208 | 7906 |
| 209 | 7955 |
| 210 | 8060 |
| 211 | 8064 |
| 212 | 8078 |
| 213 | 8113 |
| 214 | 8138 |
| 215 | 8177 |
| 216 | 8186 |
| 217 | 8245 |
| 218 | 8249 |
| 219 | 8252 |
| 220 | 8273 |
| 221 | 8276 |
| 222 | 8283 |
| 223 | 8285 |
| 224 | 8291 |
| 225 | 8304 |
| 226 | 8330 |
| 227 | 8355 |
| 228 | 8416 |
| 229 | 8426 |
| 230 | 8435 |
| 231 | 8450 |
| 232 | 8469 |
| 233 | 8695 |
| 234 | 8791 |
| 235 | 8835 |
| 236 | 8944 |
| 237 | 8966 |
| 238 | 9015 |
| 239 | 9075 |
| 240 | 9078 |
| 241 | 9091 |
| 242 | 9113 |
| 243 | 9118 |
| 244 | 9123 |
| 245 | 9127 |
| 246 | 9139 |
| 247 | 9160 |
| 248 | 9232 |
| 249 | 9239 |
| 250 | 9252 |
| 251 | 9270 |
| 252 | 9274 |
| 253 | 9313 |
| 254 | 9325 |
| 255 | 9347 |
| 256 | 9350 |
| 257 | 9379 |
| 258 | 9385 |
| 259 | 9387 |
| 260 | 9397 |
| 261 | 9401 |
| 262 | 9407 |
| 263 | 9416 |
| 264 | 9423 |
| 265 | 9437 |
| 266 | 9457 |
| 267 | 9484 |
| 268 | 9486 |
| 269 | 9501 |
| 270 | 9513 |
| 271 | 9550 |
| 272 | 9562 |
| 273 | 9621 |
| 274 | 9640 |
| 275 | 9650 |
| 276 | 9663 |
| 277 | 9667 |
| 278 | 9678 |
| 279 | 9685 |
| 280 | 9698 |
| 281 | 9704 |
| 282 | 9720 |
| 283 | 9730 |
| 284 | 9737 |
| 285 | 9739 |
| 286 | 9748 |
| 287 | 9765 |
| 288 | 9768 |
| 289 | 9771 |
| 290 | 9788 |
| 291 | 9792 |
| 292 | 9800 |
| 293 | 9819 |
| 294 | 9849 |
| 295 | 9858 |
| 296 | 9861 |
| 297 | 9864 |
| 298 | 9870 |
| 299 | 9879 |
| 300 | 9882 |
| 301 | 9885 |
| 302 | 9891 |
| 303 | 9894 |
| 304 | 9898 |
| 305 | 9902 |
| 306 | 9910 |
| 307 | 9920 |
| 308 | 9924 |
| 309 | 9927 |
| 310 | 9929 |
| 311 | 9934 |
| 312 | 9939 |
| 313 | 9948 |
| 314 | 9952 |
| 315 | 9966 |
| 316 | 9968 |
| 317 | 9973 |
| 318 | 9978 |
| 319 | 9995 |
| 320 | 10000 |
| 321 | 10006 |
| 322 | 10050 |
| 323 | 10054 |
| 324 | 10057 |
| 325 | 10072 |
| 326 | 10075 |
| 327 | 10078 |
| 328 | 10082 |
| 329 | 10090 |
| 330 | 10093 |
| 331 | 10118 |
| 332 | 10360 |
| 333 | 10370 |
| 334 | 10401 |
| 335 | 10406 |
| 336 | 10408 |
| 337 | 10446 |
| 338 | 10467 |
| 339 | 10470 |
| 340 | 10475 |
| 341 | 10506 |
| 342 | 10509 |
| 343 | 10541 |
| 344 | 10548 |

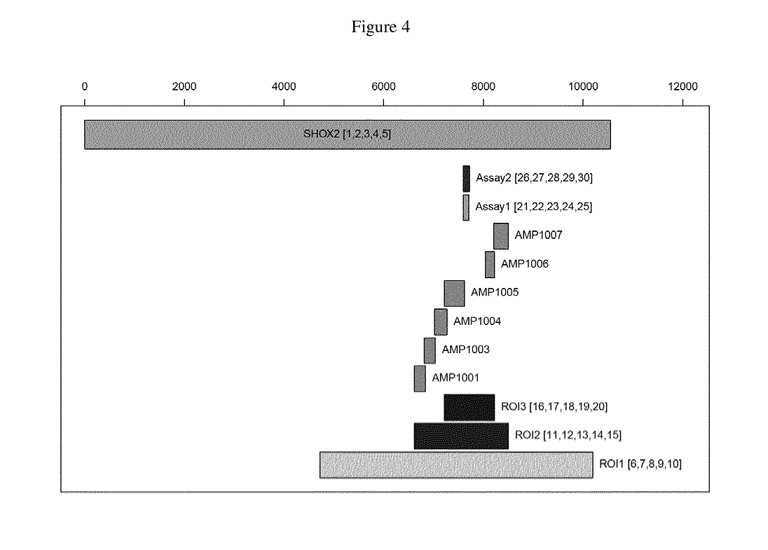

A hypermethylation in shox2 according to the invention is preferably determined at 1 or more, 2 or more, 3 or more, 4 or more, 5 or more, 6 or more, 10 or more, 15 or more or 30 or more of the CpG sites comprised in SEQ ID NO: 1, more preferably in SEQ ID NO: 6, even more preferably in SEQ ID NO: 11, and most preferably in SEQ ID NO: 16. Particular sequence regions for the determination are also the regions according to SEQ ID NO: 21 and 26 as well as nt 6611 to 6837 of SEQ ID NO: 1, nt 6814 to 7037 of SEQ ID NO: 1, nt 7016 to 7270 of SEQ ID NO: 1, nt 7215 to 7618 of SEQ ID NO: 1, nt 8038 to 8222 of SEQ ID NO: 1, and nt 8207 to 8501 of SEQ ID NO: 1. In a preferred embodiment, the methylation of CpG sites that are co-methylated (preferably within SEQ ID NO: 1) in cancer, in particular in lung cancer, may also or instead be determined. Detailed information on the above sequences and regions and their relationship can be found in the following Table 3. The same is visualized in FIG. 4.

TABLE 3
Positions of SHOX2 and regions of interest (ROIs), bisulfite
sequenced regions (see Example 2) and assays
(see Example 1) in SHOX2 and CpGs in SHOX2 sequence.

| | | | | | CpGs in |
|---|---|---|---|---|---|
| Name | Seq Ids | in | from | to | SHOX2 |
| SHOX2 | 1, 2, 3, 4, 5 | Chrom. 3 | 158095954 | 158106503 | 1 to 344 |
| ROI1 | 6, 7, 8, 9, 10 | SHOX2 | 4722 | 10194 | 113 to 331 |
| R012 | 11, 12, 13, | SHOX2 | 6611 | 8501 | 131 to 232 |
| | 14, 15 | | | | |
| R0I3 | 16, 17, 18, | SHOX2 | 7215 | 8222 | 163 to 216 |
| | 19, 20 | | | | |
| AMP1001 | | SHOX2 | 6611 | 6837 | 131 to 139 |
| AMP1003 | | SHOX2 | 6814 | 7037 | 140 to 151 |
| AMP1004 | | SHOX2 | 7016 | 7270 | 152 to 163 |
| AMP1005 | | SHOX2 | 7215 | 7618 | 163 to 182 |
| AMP1006 | | SHOX2 | 8038 | 8222 | 210 to 216 |
| AMP1007 | | SHOX2 | 8207 | 8501 | 217 to 232 |
| Assay1 | 21, 22, 23, | SHOX2 | 7597 | 7708 | 183 to 193 |
| | 24, 25 | | | | |
| Assay2 | 26, 27, 28, | SHOX2 | 7597 | 7720 | 183 to 193 |
| | 29, 30 | | | | |

Accordingly, the invention also relates to a nucleic acid comprising at least 16 contiguous nucleotides of a sequence selected from the group consisting of SEQ ID NOs 2-5, 7-10, 12-15, 17-20, 22-25 and 27-30, in particular for the use in monitoring a cancer under treatment, predicting the effect of a cancer treatment, identifying a patient as a responder to a cancer treatment, identifying a patient as a non-responder to a cancer treatment and/or treating cancer as described herein, in particular in the methods according to aspects 1 to 5 described herein, respectively.

The term “genomic DNA” as used herein refers to chromosomal DNA and is used to distinguish from coding DNA. As such, it includes exons, introns as well as regulatory sequences, in particular promoters, belonging to a gene.

The term “test sample” as used herein refers to biological material isolated from a patient. The test sample comprises cells of the cancer or free genomic DNA from cancer cells, preferably circulating genomic DNA from cancer cells. It can be derived from any suitable tissue or biological fluid such as blood, saliva, plasma, serum, urine, cerebrospinal liquid (CSF), feces, a buccal or buccal-pharyngeal swab, a surgical specimen, a specimen obtained from a biopsy, or a tissue sample embedded in paraffin. Methods for deriving samples are well known to those skilled in the art. Preferably, the sample is a tumor biopsy or a body liquid sample. The body liquid sample is preferably blood, blood serum, blood plasma, or urine. Most preferably, it is blood plasma. In case the cancer is lung cancer, it is also envisaged that the sample comprises matter derived from bronchoscopy, including bronchial lavage, bronchial alveolar lavage, bronchial brushing or bronchial abrasion, or from sputum or saliva.

The term “patient” as used herein refers to an individual, such as a human, a non-human primate (e.g. chimpanzees and other apes and monkey species); farm animals, such as birds, fish, cattle, sheep, pigs, goats and horses; domestic mammals, such as dogs and cats; laboratory animals including rodents, such as mice, rats and guinea pigs. The term does not denote a particular age or sex. In a particular embodiment of the invention, the subject is a mammal. In a preferred embodiment of the invention, the subject is a human. In one embodiment, the patient has not been treated before for the cancer to be treated.

The term “subsequently” as used herein refers to the samples being taken at different time points, i.e. one after the other. Preferably, the time between samples being taken is at least 1, 2, 3, 4, 5, 6, or 7 days and up to 3, 2 or 1 month(s) or up to 12, 8, 4, 2 or 1 week, more preferably 2-20, 4-16 or 7-12 days. The term may also mean that the samples are taken at treatment specific events, e.g. before and/or after every treatment cycle or drug/therapy administration.

In a second aspect, the present invention relates to a method for predicting the effect of a cancer treatment, comprising determining the amount of hypermethylated shox2 genomic DNA in a first and one or more further test samples of a cancer patient taken subsequently, wherein a change in or a stagnation of the amount of hypermethylated shox2 genomic DNA indicates the effect of the treatment.

The term “predicting the effect of a cancer treatment” as used herein refers to the expected outcome of the cancer disease in response to the treatment and relates to the assessment of its state of development, progression, or of its regression, and/or the prognosis of the course of the cancer in the future. As will be understood by persons skilled in the art, such assessment normally may not be correct for 100% of the patients, although it preferably is correct. The term, however, requires that a correct prediction can be made for a statistically significant part of the subjects. Whether a part is statistically significant can be determined easily by the person skilled in the art using several well known statistical evaluation tools, for example, determination of confidence intervals, determination of p values, Student's t-test, Mann-Whitney test, etc. Details are provided in Dowdy and Wearden, Statistics for Research, John Wiley & Sons, New York 1983. The preferred confidence intervals are at least 50%, at least 60%, at least 70%, at least 80%, at least 90%, at least 95%. The p values are preferably 0.05, 0.01, or 0.005. The prediction of the treatment effect can be done using any assessment criterion used in oncology and known to the person skilled in the art. The assessment parameters useful for describing the progression of a disease include, for example, relapse-free survival (RFS). Generally, the effect of the treatment can be assessed by determining the tumor size and/or the number of cancer cells. A stagnation and/or a decrease, preferably a decrease, of one or both would be a positive effect of the cancer treatment, wherein an increase of one or both would be a negative effect of the cancer treatment.

The term “change in the amount” as used herein refers to an increase or decrease, preferably a substantial or non-substantial decrease as defined below.

The term “stagnation of the amount” as used herein refers to a stagnation as defined below.

In a third aspect, the present invention relates to a method for identifying a patient as a responder to a cancer treatment, comprising determining the amount of hypermethylated shox2 genomic DNA in a first and one or more further test samples of a cancer patient taken subsequently, wherein a substantial decrease in the amount of hypermethylated shox2 genomic DNA indicates a response to the treatment.

The term “responder to a cancer treatment” as used herein refers to a patient on which the treatment has an effect as defined above. Preferably, a responder shows complete or partial remission of the tumor.

The term “decrease” as used herein refers to a lower amount of hypermethylated shox2 genomic DNA in a further test sample compared to the first test sample and/or one or more previously taken further samples.

The term “substantial decrease” as used herein refers generally to a decrease which is found in a group of responders to a cancer treatment compared to a decrease which is or may be found in a group of non-responders to a cancer treatment. A threshold value can be determined by the skilled person by monitoring the treatment of a cohort of patients according to the invention. In a particular embodiment, a “substantial decrease” can mean one or more of:

a decrease as defined herein, preferably with respect to the first sample and/or one or more previously taken further samples (more preferably a second, third, fourth, and/or fifth sample) in at least 1, 2, 3, 4, 5, 6, 7, 8, 9, or 10 further samples, or in all further samples compared only to the first sample, preferably starting at further sample 1, 2, 3, 4 or 5;
a decrease to or under a defined threshold or the limit of detection, wherein the defined threshold preferably is between PMR values of 0.1-2% and the limit of detection preferably is between PMR values, as defined herein, of 0.1%-5%, 0.1%-4%, 0.1%-3%, 0.1%-2%, 0.1%-1%, or more preferably is a PMR value of <0.1%
a decrease to or under a defined threshold of the limit of detection as defined above in at least 1, 2, 3, 4, 5, 6, 7, 8, 9 or 10 further samples;
a decrease from a minimum baseline amount of hypermethylated shox2 genomic DNA for the first sample taken before treatment that is within a range starting at the limit of detection of the detection method and preferably ending at about 125%, 150%, 175%, 200%, 250%, 300%, 400%, 500%, or more (e.g. 1000%, 1500% or 5000%) of the limit of detection, to an amount of hypermethylated shox2 genomic DNA below that level in one or more further samples, preferably at least 2, 3, 4, or 5 further samples, to a level that is distinguishable by the detection method used from the baseline amount or is below the limit of detection.
a decrease from a discrete PMR value between 5 and 0.1% for the first sample (the lower range dependent on the limit of detection, i.e. the technical sensitivity of the detection method) to a PMR value below this discrete value in one or more further samples, preferably at least 2, 3, 4, or 5 further samples, or to a PMR value below the discrete value in one and all subsequent further samples (sustained reduction); or
a decrease from a PMR value≥1% for the first sample to a PMR value<1% in one or more further samples, preferably at least 2, 3, 4, or 5 further samples, or to a PMR value<1% in all subsequent further samples (sustained reduction); or
a decrease by at least 10, 20, 30, 40, 50, 60, 70, 80, 90, 95, 97 or 99% in at least 1, 2, 3, 4, 5, 6, 7, 8, 9 or 10 further samples compared to the first sample or one or more (e.g. 2, 3, 4, 5, 6, 7, 8, 9 or 10) previously taken further samples;

In a fourth aspect, the present invention relates to a method for identifying a patient as a non-responder to a cancer treatment, comprising determining the amount of hypermethylated shox2 genomic DNA in a first and one or more further test samples of a cancer patient taken subsequently, wherein an increase, a stagnation or a non-substantial decrease in the amount of hypermethylated shox2 genomic DNA indicates a non-response to the treatment.

The term “non-responder to a cancer treatment” as used herein refers to a patient on which the treatment has no effect as defined above. Preferably, a non-responder shows progression of the tumor.

The term “increase” as used herein refers to a higher amount of hypermethylated shox2 genomic DNA in a further test sample compared to the first test sample and/or one or more previously taken further samples.

The term “stagnation” as used herein refers to an amount which has not changed, preferably not changed statistically significant between a further test sample compared to the first test sample and/or one or more earlier taken further samples.

The term “non-substantial decrease” as used herein refers to a decrease which is not a substantial decrease as defined above.

Statistical significance, as used herein, can be determined easily by the person skilled in the art using several well known statistical evaluation tools, for example, determination of confidence intervals, determination of p values, Student's t-test, Mann-Whitney test, etc. Details are provided in Dowdy and Wearden, Statistics for Research, John Wiley & Sons, New York 1983. The preferred confidence intervals are at least 50%, at least 60%, at least 70%, at least 80%, at least 90%, at least 95%. The p values are preferably 0.05, 0.01, or 0.005.

In one embodiment of the methods of aspect one to four of the invention, the first test sample is taken before the start of the treatment and the one or more further test samples are taken during the course of the treatment, i.e. after the start of the treatment. The patient may or may not have been treated with a different treatment before said start of the treatment, preferably he has not been treated before.

In a fifth aspect, the present invention relates to a method for treating cancer, comprising the steps:
(i) determining the amount of hypermethylated shox2 genomic DNA in a first test sample of a cancer patient;
(ii) treating said patient with a first treatment regimen comprising one or more anti-cancer agents or therapies,
(iii) determining the amount of hypermethylated shox2 genomic DNA in one or more subsequently taken further test samples of said patient;
(iv) optionally repeating steps (ii) and (iii) one or more times;
(v) continuing treating the patient with the first treatment regimen if there is a substantial decrease in the amount of hypermethylated shox2 genomic DNA, or terminating treating the patient with the first treatment regimen and treating the patient instead with a second treatment regimen comprising one or more anti-cancer agents or therapies not comprised in the first treatment regimen if there is an increase, a stagnation or a non-substantial decrease in the amount of hypermethylated shox2 genomic DNA.

The term “treatment regimen” as used herein refers to how the patient is treated in view of the disease and available procedures and medication. Non-limiting examples of cancer treatment regimes are chemotherapy, surgery and/or irradiation or combinations thereof. It particular, it refers to administering one or more anti-cancer agents or therapies as defined below.

The term “anti-cancer agent or therapy” as used herein refers to chemical, physical or biological agents or therapies, or surgery, including combinations thereof, with antiproliferative, antioncogenic and/or carcinostatic properties.

A chemical anti-cancer agent or therapy may be selected from the group consisting of alkylating agents, antimetabolites, plant alkaloyds and terpenoids and topoisomerase inhibitors. Preferably, the alykylating agents are platinum-based compounds. In one embodiment, the platinum-based compounds are selected from the group consisting of cisplatin, oxaliplatin, eptaplatin, lobaplatin, nedaplatin, carboplatin, iproplatin, tetraplatin, lobaplatin, DCP, PLD-147, JM1 18, JM216, JM335, and satraplatin.

A physical anti-cancer agent or therapy may be selected from the group consisting of radiation therapy (e.g. curative radiotherapy, adjuvant radiotherapy, palliative radiotherapy, teleradiotherapy, brachytherapy or metabolic radiotherapy), phototherapy (using, e.g. hematoporphoryn or photofrin II), and hyperthermia.

Surgery may be a curative resection, palliative surgery, preventive surgery or cytoreductive surgery. Typically, it involves an excision, e.g. intracapsular excision, marginal, extensive excision or radical excision as described in Baron and Valin (Rec. Med. Vet, Special Canc. 1990; 11(166):999-1007).

A biological anti-cancer agent or therapy may be selected from the group consisting of antibodies (e.g. antibodies stimulating an immune response destroying cancer cells such as retuximab or alemtuzubab, antibodies stimulating an immune response by binding to receptors of immune cells an inhibiting signals that prevent the immune cell to attack “own” cells, such as ipilimumab, antibodies interfering with the action of proteins necessary for tumor growth such as bevacizumab, cetuximab or panitumumab, or antibodies conjugated to a drug, preferably a cell-killing substance like a toxin, chemotherapeutic or radioactive molecule, such as Y-ibritumomab tiuxetan, I-tositumomab or ado-trastuzumab emtansine), cytokines (e.g. interferons or interleukins such as INF-alpha and IL-2), vaccines (e.g. vaccines comprising cancer-associated antigens, such as sipuleucel-T), oncolytic viruses (e.g. naturally oncolytic viruses such as reovirus, Newcastle disease virus or mumps virus, or viruses genetically engineered viruses such as measles virus, adenovirus, vaccinia virus or herpes virus preferentially targeting cells carrying cancer-associated antigens such as EGFR or HER-2), gene therapy agents (e.g. DNA or RNA replacing an altered tumor suppressor, blocking the expression of an oncogene, improving a patient's immune system, making cancer cells more sensitive to chemotherapy, radiotherapy or other treatments, inducing cellular suicide or conferring an anti-angiogenic effect) and adoptive T cells (e.g. patient-harvested tumor-invading T-cells selected for antitumor activity, or patient-harvested T-cells genetically modified to recognize a cancer-associated antigen).

In one embodiment, the one or more anti-cancer drugs is/are selected from the group consisting of Abiraterone Acetate, ABVD, ABVE, ABVE-PC, AC, AC-T, ADE, Ado-Trastuzumab Emtansine, Afatinib Dimaleate, Aldesleukin, Alemtuzumab, Aminolevulinic Acid, Anastrozole, Aprepitant, Arsenic Trioxide, Asparaginase Erwinia chrysanthemi, Axitinib, Azacitidine, BEACOPP, Belinostat, Bendamustine Hydrochloride, BEP, Bevacizumab, Bexarotene, Bicalutamide, Bleomycin, Bortezomib, Bosutinib, Brentuximab Vedotin, Busulfan, Cabazitaxel, Cabozantinib-S-Malate, CAFCapecitabine, CAPDX, Carboplatin, CARBOPLATIN-TAXOL, Carfilzomib, Carmustine, Carmustine Implant, Ceritinib, Cetuximab, Chlorambucil, CHLORAMBUCIL-PREDNISONE, CHOP, Cisplatin, Clofarabine, CMF, COPP, COPP-ABV, Crizotinib, CVP, Cyclophosphamide, Cytarabine, Cytarabine, Liposomal, Dabrafenib, Dacarbazine, Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, Doxorubicin Hydrochloride Liposome, Eltrombopag Olamine, Enzalutamide, Epirubicin Hydrochloride, EPOCH, Eribulin Mesylate, Erlotinib Hydrochloride, Etoposide Phosphate, Everolimus, Exemestane, FEC, Filgrastim, Fludarabine Phosphate, Fluorouracil, FU-LV, Fulvestrant, Gefitinib, Gemcitabine Hydrochloride, GEMCITABINE-CISPLATIN, GEMCITABINE-OXALIPLATIN, Gemtuzumab Ozogamicin, Glucarpidase, Goserelin Acetate, HPV Bivalent Vaccine, Recombinant HPV Quadrivalent Vaccine, Hyper-CVAD, Ibritumomab Tiuxetan, Ibrutinib, ICE, Idelalisib, Ifosfamide, Imatinib, Mesylate, Imiquimod, Iodine 131 Tositumomab and Tositumomab, Ipilimumab, Irinotecan Hydrochloride, Ixabepilone, Lapatinib Ditosylate, Lenalidomide, Letrozole, Leucovorin Calcium, Leuprolide Acetate, Liposomal Cytarabine, Lomustine, Mechlorethamine Hydrochloride, Megestrol Acetate, Mercaptopurine, Mesna, Methotrexate, Mitomycin C, Mitoxantrone Hydrochloride, MOPP, Nelarabine, Nilotinib, Obinutuzumab, Ofatumumab, Omacetaxine Mepesuccinate, OEPA, OFF, OPPA, Oxaliplatin, Paclitaxel, Paclitaxel Albumin-stabilized Nanoparticle Formulation, PAD, Palifermin, Palonosetron Hydrochloride, Pamidronate Disodium, Panitumumab, Pazopanib Hydrochloride, Pegaspargase, Peginterferon Alfa-2b, Pembrolizumab, Pemetrexed Disodium, Pertuzumab, Plerixafor, Pomalidomide, Ponatinib Hydrochloride, Pralatrexate, Prednisone, Procarbazine Hydrochloride, Radium 223 Dichloride, Raloxifene Hydrochloride, Ramucirumab, Rasburicase, R-CHOP, R-CVP, Recombinant HPV Bivalent Vaccine, Recombinant HPV Quadrivalent Vaccine, Recombinant Interferon Alfa-2b, Regorafenib, Rituximab, Romidepsin, Romiplostim, Ruxolitinib Phosphate, Siltuximab, Sipuleucel-T, Sorafenib Tosylate, STANFORD V, Sunitinib Malate, TAC, Talc, Tamoxifen Citrate, Temozolomide, Temsirolimus, Thalidomide, Topotecan Hydrochloride, Toremifene, Tositumomab and I 131 Iodine Tositumomab, TPF, Trametinib, Trastuzumab, Vandetanib, VAMP, VeIP, Vemurafenib, Vinblastine Sulfate, Vincristine Sulfate, Vincristine Sulfate Liposome, Vinorelbine Tartrate, Vismodegib, Vorinostat, XELOX, Ziv-Aflibercept, and Zoledronic Acid.

Generally, in the methods of aspect one to five of the invention, the amount of hypermethylated shox2 genomic DNA in the sample is determined by a process selected from the group consisting of COBRA, restriction ligation-mediated PCR, Ms-SNuPE, ion-pair reverse-phase high performance liquid chromatography, denaturing high performance liquid chromatography, any bisulfite sequencing method, e.g. direct bisulfite sequencing with the Sanger method or sequencing methods of the 2^{nd} or 3^{rd} generation (NexGen sequencing), or any pyrosequencing method, DNA sequencing methods that can per se distinguish between methylated and unmethylated cytosines (e.g. using Nanopores and/or enzymes used as sensors), MALDI-TOF, QM™ or real-time PCR, preferably MethyLight™ or HeavyMethyl™ or a combination thereof

The term “COBRA” (Combined Bisulfite Restriction Analysis) refers to the art-recognized methylation assay described by Xiong and Laird, Nucleic Acids Res. 25:2532-2534, 1997, which is herein incorporated by reference.

The term “restriction ligation-mediated PCR” refers to the art-recognized methylation assay described by Steigerwald et al. (Nucleic Acids Res. Mar. 25, 1990; 18(6): 1435-1439), which is herein incorporated by reference.

The term “ion-pair reverse-phase high performance liquid chromatography” refers to the art-recognized methylation assay described by Matin et al. (Hum Mutat. 2002 Oct; 20(4):305-11), which is herein incorporated by reference.

The term “denaturing high performance liquid chromatography” refers to the art-recognized methylation assay described by Hattori et al. (Genet Test Mol Biomarkers. 2009 Oct; 13(5):623-30), which is herein incorporated by reference.

The term “pyrosequencing” refers to the art-recognized methylation assay described by Colella et al. (BioTechniques 35:146-150, 2003), which is herein incorporated by reference.

The term “MALDI-TOF” refers to the art-recognized methylation assay described by Ehrich et al. (PNAS vol. 102, no. 44, 15785-15790), which is herein incorporated by reference.

The term “Ms-SNuPE” (Methylation-sensitive Single Nucleotide Primer Extension) refers to a quantitative method for assessing methylation differences at specific CpG sites based on bisulfite treatment of DNA, followed by single-nucleotide primer extension (Gonzalgo and Jones, Nucleic Acids Res. 25:2529-2531, 1997, herein incorporated by reference). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

The term “QM™” refers to a quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides.

The term “MethyLight™” refers to a high-throughput quantitative methylation assay that utilizes fluorescence-based real-time PCR technology that requires no further manipulations after the PCR step (Eads et al., Cancer Res. 59:2302-2306, 1999, incorporated herein by reference). Briefly, the MethyLight™ process begins with a mixed sample of genomic DNA that is converted, e.g. in a sodium bisulfite reaction, to a mixed pool of methylation-dependent sequence differences according to standard procedures (the bisulfite process converts unmethylated cytosine residues to uracil). Fluorescence-based PCR is then performed in a “biased” (with PCR primers that overlap known CpG dinucleotides) reaction. Sequence discrimination can occur both at the level of the amplification process and at the level of the fluorescence detection process. It may be used as a quantitative test for methylation patterns in the genomic DNA sample, wherein sequence discrimination occurs at the level of probe hybridization. In this quantitative version, the PCR reaction provides for a methylation specific amplification in the presence of a fluorescent probe that overlaps a particular putative methylation site. An unbiased control for the amount of input DNA is provided by a reaction in which neither the primers, nor the probe overlie any CpG dinucleotides.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

In a preferred embodiment of the methods of aspect one to five of the invention, determining the amount of hypermethylated shox2 genomic DNA comprises a step of converting, in the genomic DNA, cytosine unmethylated at the 5-position to uracil or another base that does not hybridize to guanine. This step of converting involves chemically treating the DNA in such a way that all or substantially all of the unmethylated cytosine bases are converted to uracil bases, or another base which is dissimilar to cytosine in terms of base pairing behaviour, while the 5-methylcytosine bases remain unchanged. The conversion of unmethylated, but not methylated, cytosine bases within the DNA sample is conducted with a converting agent. The term “converting agent” as used herein relates to a reagent capable of converting an unmethylated cytosine to uracil or to another base that is detectably dissimilar to cytosine in terms of hybridization properties. The converting agent is preferably a bisulfite such as disulfite, or hydrogen sulfite. The reaction is performed according to standard procedures (Frommer et al., 1992, Proc Natl Acad Sci USA 89:1827-31; Olek, 1996, Nucleic Acids Res 24:5064-6; EP 1394172). It is also possible to conduct the conversion enzymatically, e.g. by use of methylation specific cytidine deaminases. Most preferably, the converting agent is sodium bisulfite or bisulfite.

In a more preferred embodiment, determining the amount of hypermethylated shox2 genomic DNA comprises a further step of amplifying at least a fragment of shox2 genomic DNA in a methylation dependent manner. The fragment comprises at least the region or the CpG sites for which the amount of hypermethylation is to be determined. For practical reasons, the fragment is at least 50, 100, 150, 200 or 300 base pairs (bp) long and/or not longer than 500, 600, 700, 800, 900 or 1000 bp. The amplification is preferably performed by methylation-specific PCR (i.e. an amplificate is produced depending on whether one or more CpG sites are converted or not), more preferably using primers which are methylation-specific (i.e. hybridize to converted or non-converted CpG sites) or not methylation-specific, but specific to bisulfite-converted DNA (i.e. hybridize to converted DNA not comprising any CpG sites). In case of the latter, methylation-specificity is achieved, e.g., by using methylation-specific blocker oligonucleotides, which hybridize to converted or non-converted CpG sites and thereby terminate the PCR polymerization. In a most preferred embodiment, the step of amplifying comprises a real-time PCR, in particular MethyLight™ or HeavyMethyl™ MethyLight™ as described above.

The term “hybridization”, when used with respect to an oligonucleotide, is to be understood as a bond of an oligonucleotide to a complementary sequence along the lines of the Watson-Crick base pairings in the sample DNA, forming a duplex structure, under moderate or stringent hybridization conditions. When it is used with respect to a single nucleotide or base, it refers to the binding according to Watson-Crick base pairings, e.g. C-G, A-T and A-U. Stringent hybridization conditions involve hybridizing at 68° C. in 5×SSC/5× Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderate conditions involve washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

In another preferred embodiment of the methods of aspect one to five of the invention, determining the amount of hypermethylated shox2 genomic DNA in the test sample comprises normalizing for the amount of total DNA in the sample. Normalizing for the amount of total DNA in the test sample preferably comprises calculating the ratio of the amount of hypermethylated shox2 genomic DNA and the amount of genomic DNA of a reference gene. The reference gene is preferably a housekeeping gene.

A housekeeping gene is a constitutively expressed gene involved in or required for the maintenance of basic cellular function and is expressed in all cells of an organism under normal and patho-physiological conditions. In humans alone, there are more than 2000 housekeeping genes (see Chang et al., PLoS ONE 6(7): e22859. doi:10.1371/journal.pone.0022859, which is hereby incorporated by reference), which may all be used according to the invention. None-limiting examples are Human acidic ribosomal protein (HuPO), β-Actin (BA), Cyclophylin (CYC), Glyceraldehyde-3-phosphate dehydrogenase (GAPDH), Phosphoglycerokinase (PGK), β2-Microglobulin (B2M), β-Glucuronidase (GUS), Hypoxanthine phosphoribosyltransferase (HPRT), Transcription factor IID TATA binding protein (TBP), Transferrin receptor (TfR), Elongation factor-1-α (EF-1-α), Metastatic lymph node 51 (MLN51) and Ubiquitin conjugating enzyme (UbcH5B).

The amount of hypermethylated shox2 genomic DNA in the test sample, in a preferred embodiment of the methods of aspect one to five of the invention, is the proportion of hypermethylated shox2 genomic DNA relative to the amount of hypermethylated shox2 genomic DNA in a reference sample comprising substantially fully methylated genomic DNA. Preferably, determining the proportion of hypermethylated shox2 genomic DNA comprises determining the amount of hypermethylated shox2 genomic DNA of a reference gene in a reference sample, and dividing the ratio derived from the test sample by the corresponding ratio derived from the reference sample. The proportion can be expressed as a percentage or PMR as defined below by multiplying the result of the division by 100.

Alternatively, in particular if the amount of hypermethylated shox2 genomic DNA is determined by real-time PCR, it may be calculated by using the cycle threshold (Ct) values for shox2 and a housekeeping gene (hkg) from samples of patients and the reference (ref) sample (methylated at least at the shox2 locus) as follows: amount=100*x^{-((Ctshox2-Cthkg)-(Ctshox2ref-Cthkgref))}, which x preferably is between 1 to 3 and more preferably is 2.

The term “reference sample” refers to a sample comprising control DNA with known DNA concentration and known SHOX2 methylation state. The control DNA is preferably, but not necessarily, human DNA that is artificially methylated, preferably substantially fully methylated. In a preferred embodiment, the artificial methylation is achieved by using DNA-Methyltransferases. The DNA itself can be, for example, cell line DNA, plasmid DNA, artificial DNA, or combinations/mixtures thereof.

Substantially fully methylated genomic DNA preferably is DNA, particularly genomic DNA, which has all or substantially all CpG sites methylated. “Substantially all” in this respect means at least 95%, 96%, 97%, 98%, 99%, 99.5% or 99.9%. In a preferred embodiment, the methylation of all or substantially all CpG sites is achieved by treating the DNA with a CpG methyltransferase in a manner that provides for the methylation of all or substantially all CpG sites.

DNA methylated at the SHOX2 locus is preferably cell line DNA from one or more cell lines, preferably of those that are well characterized and of which the genomic SHOX2 methylation state is known and/or of which SHOX2 is known to be substantially fully methylated.

In a most preferred embodiment of the methods of aspect one to five of the invention, the amount of hypermethylated shox2 genomic DNA is expressed as a PMR value. The term “PMR”, “Percentage of Methylated Reference”, or “Percentage of fully Methylated Reference” describes the degree of methylation and is usually calculated by dividing the gene to reference ratio by the gene to completely methylated reference ratio (obtained, e.g. by CpG methyltransferase, for example SssI treatment of the normally unmethylated reference) and multiplying by 100. The determination of the PMR is described in detail in Ogino et al. (JMD May 2006, Vol. 8, No. 2), which is incorporated by reference. The PMR may alternatively be calculated with the ΔΔCt method by using the real-time PCR cycle threshold (Ct) values for shox2 and a housekeeping gene (hkg) from samples of patients and the reference (ref) sample (methylated at the shox2 locus) as follows: ΔΔCt=((Ctₛₕₒₓ2-Ct_{hkg})-(Ct_{shox2ref}-Ct_{hkgref})); PMR=100*x^{-ΔΔCt}, wherein x is the assumed PCR efficiency. Generally, the PCR efficiency is assumed to be between 1-3, preferably it is 2 or nearly 2. Preferably, PMRs indicated herein are the median PMR over at least 3, more preferably 4-8, most preferably 6 experimental repetitions or parallel experiments.

In a preferred embodiment of the methods of aspect one to five of the invention, the PMR value derived from the first test sample must be at least 1%.

In a preferred embodiment of the methods of aspect one to five of the invention, the amount of hypermethylated shox2 genomic DNA is determined in the first and one or more further test samples before a change in tumor size or in the amount of tumor cells (a) is determined, (b) would be determined, or (c) can be determined by conventional re-staging, respectively. Conventional tumor re-staging or conventional determination of tumor size is usually done by imaging tests like positron emission tomography (PET), computed tomography (CT) or magnetic resonance imaging (MRI). It is an advantage of the present invention that a response to treatment can be assessed before a change in tumor size can be detected by such imaging tests. Therefore, the amount of hypermethylated shox2 genomic DNA is determined before the imaging tests are usually carried out. In one embodiment, it is determined after 4, 3, 2 or preferably 1 treatment cycle. In another embodiment, it is determined 3, 2, or 1 months, or 12, 8, 6, 5, 4, 3, 2 or 1 weeks, or 14, 13, 12, 11, 10, 9, 8, 7, 6, 5, 4, 3, 2 or 1 days after the start of the treatment, preferably within 4 or 1-2, more preferably 2-4 weeks after the start of the treatment, most preferably as early as 6-12 days after the start of treatment.

The invention is described by way of the following examples which are to be construed as merely illustrative and not limitative of the scope of the invention.

### Example 1

### Patients

The inventors prospectively enrolled 36 patients referred to an outpatient clinic for diagnosis and treatment of lung cancer. From this group five patients were excluded from the analysis since they had received a treatment before enrollment in our study (second line). The details of the clinical data of the 31 patients who were evaluated are summarized in Table 4. The specimens for the histopathological diagnosis were obtained by bronchoscopy and/or computed tomography (CT). All but one patient received a standard platinum-based combination chemotherapy and if necessary an additional radiotherapy according to existing guidelines (Goeckenjan G, Sitter H, Thomas M, Branscheid D, Flentje M, et al. (2010)

Prevention, diagnosis, therapy, and follow-up of lung cancer. Pneumologie 64 Suppl 2: e1-164). Patient UKH 010 demonstrated an activating EGFR mutation and was treated with a TKI (Erlotinib). After three therapy cycles the patients were re-staged by physicians of the local tumor board based on repeat-CT. The study was approved by the Ethics Committee of the Universitätsklinikum Halle/Saale and all patients provided informed consent to participate in this investigation.

TABLE 4
Clinical data of the patients included in the analysis. The upper
24 patients demonstrated a PMR baseline value of ≥1%,
whereas patients 018, 019, 024, 029, 031, 034
and 038 demonstrated a baseline mSHOX2
PMR ≤1%, which is the threshold level for
technical/biological variance.

| | | | | State | |
|---|---|---|---|---|---|
| Patient | Gen- | | Survival | at end | Response |
| ID | der | Histology | [months] | of study | Group |
| UKH003 | female | adenocarcinoma | 5 | dead | Non- |
| | | | | | Responder |
| UKH005 | male | small-cell lung | 2 | dead | Non- |
| | | cancer | | | Responder |
| UKH007 | male | small-cell lung | 27 | alive | Responder |
| | | cancer | | | |
| UKH009 | female | small-cell lung | 1 | dead | Non- |
| | | cancer | | | Responder |
| UKH011 | male | large-cell lung | 1 | dead | Non- |
| | | cancer | | | Responder |
| UKH012 | male | small-cell lung | 6 | dead | Responder |
| | | cancer | | | |
| UKH014 | male | undifferentiated | 5 | dead | Non- |
| | | | | | Responder |
| UKH015 | male | adenocarcinoma | 7 | dead | Responder |
| UKH016 | male | undifferentiated | 8 | dead | Non- |
| | | | | | Responder |
| UKH017 | female | adenocarcinoma | 3 | dead | Non- |
| | | | | | Responder |
| UKH018 | male | squamous-cell | 3 | dead | Non- |
| | | lung cancer | | | Responder |
| UKH019 | male | adenocarcinoma | 2 | dead | Non- |
| | | | | | Responder |
| UKH022 | male | undifferentiated | 22 | alive | Responder |
| UKH023 | male | undifferentiated | 8 | dead | Non- |
| | | | | | Responder |
| UKH024 | male | adenocarcinoma | 9 | dead | Non- |
| | | | | | Responder |
| UKH025 | male | small-cell lung | 12 | dead | Responder |
| | | cancer | | | |
| UKH026 | male | adenocarcinoma | 18 | alive | Responder |
| UKH027 | male | small-cell lung | 8 | dead | Non- |
| | | cancer | | | Responder |
| UKH028 | male | squamous-cell | 1 | dead | Non- |
| | | lung cancer | | | Responder |
| UKH029 | male | adenocarcinoma | 19 | alive | Responder |
| UKH030 | male | adenocarcinoma | 11 | dead | Responder |
| UKH031 | female | adenocarcinoma | 19 | alive | Responder |
| UKH033 | male | squamous-cell | 5 | dead | Non- |
| | | lung cancer | | | Responder |
| UKH034 | female | adenocarcinoma | 19 | alive | Responder |
| UKH035 | male | adenocarcinoma | 2 | dead | Non- |
| | | | | | Responder |
| UKH036 | male | adenocarcinoma | 5 | dead | Non- |
| | | | | | Responder |
| UKH038 | male | adenocarcinoma | 8 | dead | Non- |
| | | | | | Responder |
| UKH039 | female | adenocarcinoma | 5 | dead | Non- |
| | | | | | Responder |
| UKH040 | male | adenocarcinoma | 17 | alive | Responder |
| UKH041 | male | adenocarcinoma | 6 | dead | Non- |
| | | | | | Responder |
| UKH042 | male | squamous-cell | 16 | alive | Responder |
| | | lung cancer | | | |

TABLE 5
PMR values for the 31 patients. The values shown in italics refer to patients
responding to the therapy while numbers shown in regular font refer to non-responding
patients. The data from the patients with a PMR value below 1% at base line (lower
part of the Table) are included in Table 4. For the analysis of the data, only
patients with a PMR value above 1% at base line were used.

| | Time point of blood draw | | | | | | | | |
|---|---|---|---|---|---|---|---|---|---|
| Patient ID | 0 | 1 | 2 | 3 | 4 | 5 | 6 | 7 | 8 |
| UKH003 | 1.97 | 2.40 | 0.00 | 0.56 | 1.47 | 0.92 | 0.93 | 5.82 | 15.99 |
| UKH005 | 362.30 | 187.97 | 39.30 | 164.72 | 36.26 | 28.74 | 13.91 | 417.36 | 233.87 |
| UKH007 | 26.03 | 0.65 | 0.00 | 0.00 | 0.00 | 0.00 | 0.00 | 0.00 | 0.00 |
| UKH009 | 313.93 | 119.43 | 32.30 | 8.83 | 1.06 | N/A | N/A | N/A | N/A |
| UKH011 | 1.07 | 18.87 | 0.09 | 0.47 | 1.55 | N/A | N/A | N/A | N/A |
| UKH012 | 101.12 | 44.39 | 0.74 | 0.19 | 0.00 | 0.00 | 0.00 | 0.00 | 0.29 |
| UKH014 | 85.29 | 19.97 | 7.78 | 49.72 | 2.92 | 3.06 | 63.78 | 20.24 | 46.04 |
| UKH015 | 7.61 | 5.40 | 3.75 | 1.57 | 2.00 | 1.57 | 1.53 | 1.49 | 1.16 |
| UKH016 | 90.91 | 16.16 | 14.97 | 4.17 | 3.97 | 3.43 | 3.22 | 5.02 | 11.34 |
| UKH017 | 42.22 | 6.54 | 9.20 | 5.78 | 37.87 | 22.05 | 49.56 | 7.72 | 2.84 |
| UKH018 | 0.13 | 0.06 | 0.00 | 0.67 | 0.62 | 0.00 | 0.00 | 0.58 | 0.84 |
| UKH019 | 0.47 | 5.14 | 9.42 | 32.07 | 32.07 | N/A | N/A | N/A | N/A |
| UKH022 | 1.17 | 0.32 | 0.23 | 0.00 | 1.24 | 0.81 | 0.00 | 0.00 | 0.74 |
| UKH023 | 36.10 | 68.31 | 40.97 | 38.71 | 38.62 | 27.00 | 42.73 | 65.58 | 27.89 |
| UKH024 | 0.45 | 1.93 | 1.38 | 10.02 | 0.00 | 0.00 | 0.53 | 5.06 | 2.23 |
| UKH025 | 321.00 | 0.30 | 0.00 | 0.00 | 0.00 | 0.00 | 0.00 | 0.00 | 0.00 |
| UKH026 | 2.28 | 0.59 | 1.78 | 0.97 | 1.15 | 0.00 | 0.00 | 0.00 | 0.00 |
| UKH027 | 219.33 | 44.02 | 83.95 | 61.89 | 39.01 | 35.50 | 21.31 | 13.18 | 13.04 |
| UKH028 | 27.45 | 6.25 | 5.24 | 7.96 | 1.33 | N/A | N/A | N/A | N/A |
| UKH029 | 0.46 | 0.00 | 0.02 | 0.00 | 0.00 | 0.00 | 0.00 | 0.00 | 0.00 |
| UKH030 | 5.84 | 1.16 | 0.00 | 0.00 | 0.00 | 0.00 | 0.28 | 0.00 | 0.37 |
| UKH031 | 0.00 | 0.21 | 0.00 | 0.11 | 0.00 | 0.00 | 0.00 | 0.00 | 0.00 |
| UKH033 | 26.45 | 25.77 | 26.25 | 25.06 | 26.27 | 21.53 | 25.60 | 24.33 | 35.21 |
| UKH034 | 0.05 | 0.18 | 0.00 | 0.00 | 0.00 | 0.00 | 0.00 | 0.03 | N/A |
| UKH035 | 3.48 | 5.29 | 4.08 | 1.40 | 0.96 | N/A | N/A | N/A | N/A |
| UKH036 | 1.58 | 6.86 | 0.00 | 1.32 | 1.32 | 1.05 | 3.45 | 4.52 | 3.39 |
| UKH038 | 0.00 | 0.00 | 0.00 | 0.77 | 0.00 | 0.34 | 0.59 | 0.55 | 2.87 |
| UKH039 | 1.08 | 0.28 | 0.18 | 0.21 | 0.12 | 0.20 | N/A | N/A | N/A |
| UKH040 | 1.98 | 1.17 | 0.67 | 0.84 | 0.96 | 0.26 | 0.00 | 0.00 | 0.01 |
| UKH041 | 118.37 | 169.09 | 107.01 | 26.93 | 9.17 | 29.10 | 88.67 | 47.89 | 98.01 |
| UKH042 | 15.95 | 1.75 | 0.91 | 1.50 | 0.59 | 0.48 | 0.01 | 1.14 | 1.95 |

### Preparation of Plasma Samples

The inventors obtained 2×8.5 mL EDTA blood from all patients at the time of diagnosis (pre-therapy) and every time the patients were checked for their blood counts or when they received a chemotherapy treatment (usually at intervals of 7 to 10 days). The patients were followed until the end of three therapy cycles, i.e. the time of re-staging (approx. three months). The plasma was prepared by spinning the blood samples (within 1 to 2 hrs after blood drawing) for 15 min at 500× g. After careful transfer of the plasma supernatant into a new tube the sample was spun for a second time for 15 min at 2500× g. All samples were stored in 3-4 mL aliquots at −80° C. till use.

Real-Time Quantification of mSHOX2 Plasma DNA

Free-circulating DNA from 3.5 mL plasma samples was isolated and bisulfite converted using the Epi proColon Plasma Quick Kit (Epigenomics AG, Berlin, Germany). DNA isolation and bisulfite conversion was carried out following the instruction for use with minor modifications. The DNA was finally eluted from the beads with 68 μL elution buffer and each sample was analyzed with six replicates. Together with the patient samples the inventors measured a calibrator sample (i.e. 5 ng artificially methylated bisulfite converted DNA). The sensitive and quantitative qPCR analysis of mSHOX2 was carried out as previously described (Kneip C, Schmidt B, Seegebarth A, Weickmann S, Fleischhacker M, et al. (2011) SHOX2

DNA methylation is a biomarker for the diagnosis of lung cancer in plasma. J Thorac Oncol 6: 1632-1638; Dietrich D, Jung M, Puetzer S, Leisse A, Holmes E E, et al. (2013) Diagnostic and Prognostic Value of SHOX2 and SEPT9 DNA Methylation and Cytology in Benign, Paramalignant and Malignant Pleural Effusions. PLoS One 8: e84225). The following oligos were used in two assays (Assay1 and Assay2):

shox2 forward primer Assay1/2: gttttttgga tagttaggta at (SEQ ID NO: 31)
shox2 forward HeavyMethyl blocker Assay1/2: taatttttgt tttgtttgtt tgattggggt tgtatga (SEQ ID NO: 32)
shox2 reverse MSP primer Assay1: taacccgact taaacgacga (SEQ ID NO: 33)
shox2 MethyLight probe Assay1/2: ctcgtacgac cccgatcg (SEQ ID NO: 34)
shox2 reverse primer A Assay2: cctcctacct tctaaccc (SEQ ID NO: 35)
shox2 reverse HeavyMethyl blocker Assay2: acccaactta aacaacaaac ccttta (SEQ ID NO: 36)

See also Assay1 and Assay2, respectively, of Table 3. Each sample was measured in six PCR replicates and a relative methylation value (=PMR, percent methylation reference) for mSHOX2 was calculated as described before using the adapted ΔΔCT method (Kneip C, Schmidt B, Seegebarth A, Weickmann S, Fleischhacker M, et al. (2011) SHOX2 DNA methylation is a biomarker for the diagnosis of lung cancer in plasma. J Thorac Oncol 6: 1632-1638). The mSHOX2 DNA quantification was performed after all prospectively collected plasma samples were complete, i.e. making this analysis an observational study.

### Statistics

Differences of methylation levels (PMR) in blood plasma of reponders and non-responders at base line and follow-up time point 1-8 were tested using unpaired two sample Wilcox tests (Mann Whitney) given that the PMR data was not normally distributed. The p-values were Bonferroni corrected. Other descriptive data characteristics used were median and median absolute deviation (MAD). Responder Operator Characteristics (ROC) curves were used to visualize the capability of the shox2 marker to discriminate between responders and non-responders at different time points. All analyses were carried out using R (R project for Statistical Computing. (2014) http://www r-project org/Available: http://www.r-project.org/).

### Results

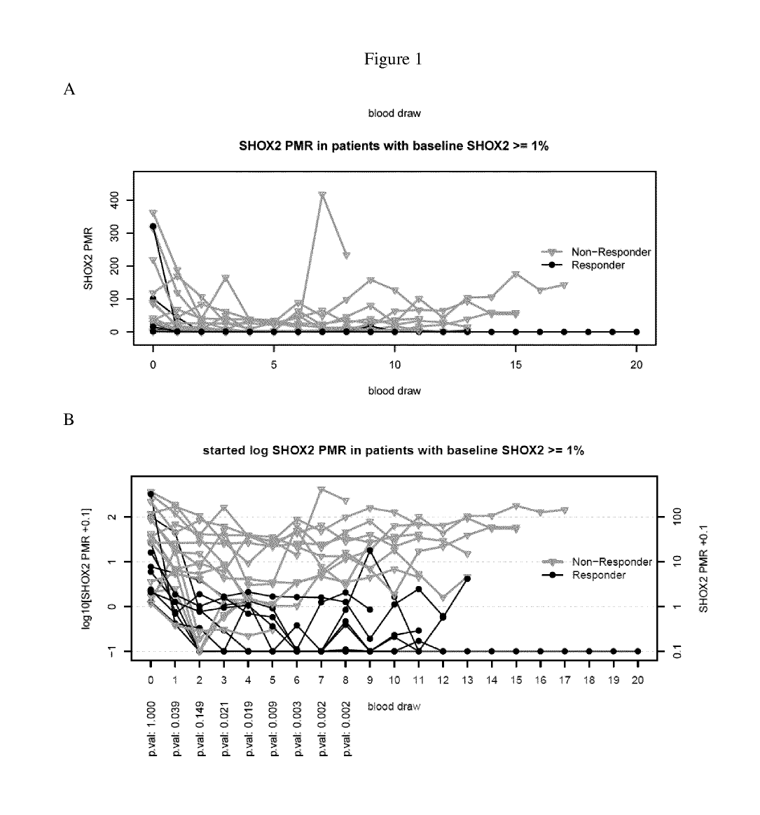

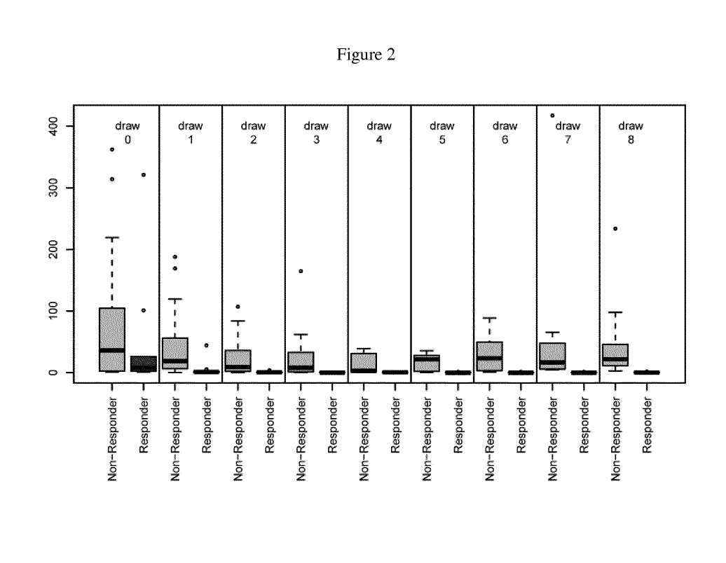

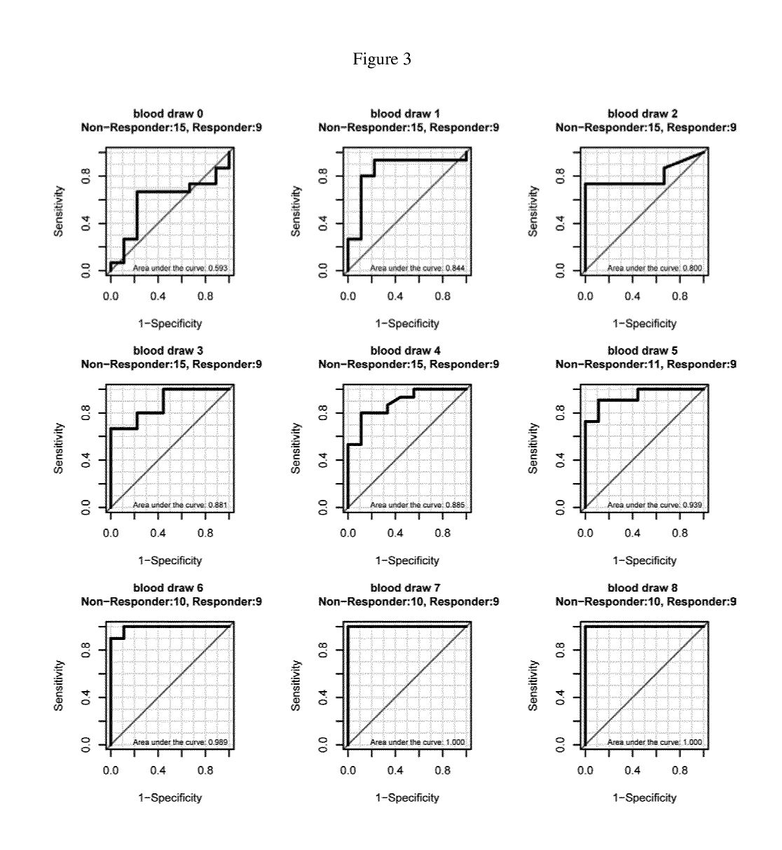

From the 36 patients who were prospectively enrolled, the clinical data for all patients were obtained and assigned into responders and non-responders, respectively. This assignment was the result of the CT-based re-staging of the local tumor board and was completely independent of the mSHOX2 analysis. Five patients were removed from the data set since they had been treated before enrollment into the study. All other 31 patients received a first-line therapy. Seven of these 31 patients demonstrated a PMR value below 1% at base line (i.e. before treatment) which is assumed to be the level of technical/biological variance. The data from all 31 patients including the 24 patients with a PMR value above 1% at base line (nine patients who responded to the therapy and 15 patients who were assigned as non-responders) which were used for the calculation of PMRs and ROC curves are summarized in Table 4. From the nine patients who responded to the therapy, three were diagnosed with SCLC, four with adenocarcinoma, one with a squamous cell carcinoma, and one was diagnosed with an undifferentiated carcinoma. In the non-responder group with PMR baseline value of ≥1%, three patients had a SCLC, six an adenocarcinoma, two patients had a squamous cell carcinoma, three patients were diagnosed with undifferentiated carcinoma and one patient had a large-cell lung carcinoma. All patients who clinically responded to the therapy demonstrated a decrease of their mSHOX2 plasma DNA (FIGS. 1 and 2). In this group a decrease of mSHOX2 DNA was seen in all nine patients already at the time of first blood draw (i.e. day 7-12 after start of therapy). In 7/9 patients who responded to the therapy the mSHOX2 level decreased to a PMR below 1% at blood draw two after therapy start but only 3/15 non-responders showed a decrease below 1%. This trend of a strong decrease for responders and only moderate or temporary decrease in most non-responders is continued: Median PMR for responders drops below 2% at blood draw 1 and below 1% at blood draw 2 and stays at the level <1%, while median PMR for non-responders is still at 18.9% at blood draw 1 and 9.2% at blood draw 2, later increases again and at blood draw 5-8 is back at values around 20% (FIG. 1, FIG. 2 and Table 5). Though the median PMR for mSHOX2 at baseline was 4.06% for the 12 responders and 26.45% for the 19 non-responders, ROC curve analysis for baseline data on all 31 patients showed that the mSHOX2 baseline values did not discriminate between responders and non-responders (area under the curve 0.581, data not shown, p-value from Wilcox test: 0.465). In contrast, ROC curve analyses for the discrimination of the two patient populations that had at least 1% PMR at baseline obtained with data after the start of the therapy demonstrated a high sensitivity and specificity already starting with blood draw one with an AUC of 0.844 which increased to 1.000 at blood draw 7 (FIG. 3). While the Bonferroni corrected p-value for a Wilcox test for responders vs. non-responders at base line is 1.0, p-values decrease thereafter and except for at time point 2 (p-value of 0.149) are <=0.05 for blood draw 1-8 (see FIG. 1).

These data illustrate that it is possible to discriminate responding from non-responding patients based on their mSHOX2 values already at the first and second blood draw with a very high sensitivity and specificity.

### Example 2

This example shows that hypermethylation in SHOX2 in lung cancer is not restricted to the specific CpG sites of covered by the real-time PCR assays of Example 1. In order to characterize SHOX2 DNA-methylation in lung cancer and healthy lung on individual CpG level, the neighbourhood of the loci of these assays was investigated broadly by direct bisulfite sequencing (DBS, Lewin J, Schmitt A O, Adorjan P, Hildmann T, Piepenbrock C. Quantitative DNA methylation analysis based on four-dye trace data from direct sequencing of PCR amplificates. Bioinformatics. 2004 Nov. 22; 20(17):3005-12. Epub 2004 Jul9. PM ID: 15247106; Lewin J Method development for quantitative methylation analysis by direct bisulfite sequencing, raw data processing 2007, URN: urn:nbn:de:bsz:291-scidok-14308). The locations of the bisulfite amplificates that span more than a kilobase around the assay locations are shown in Table 3 and in FIG. 4.

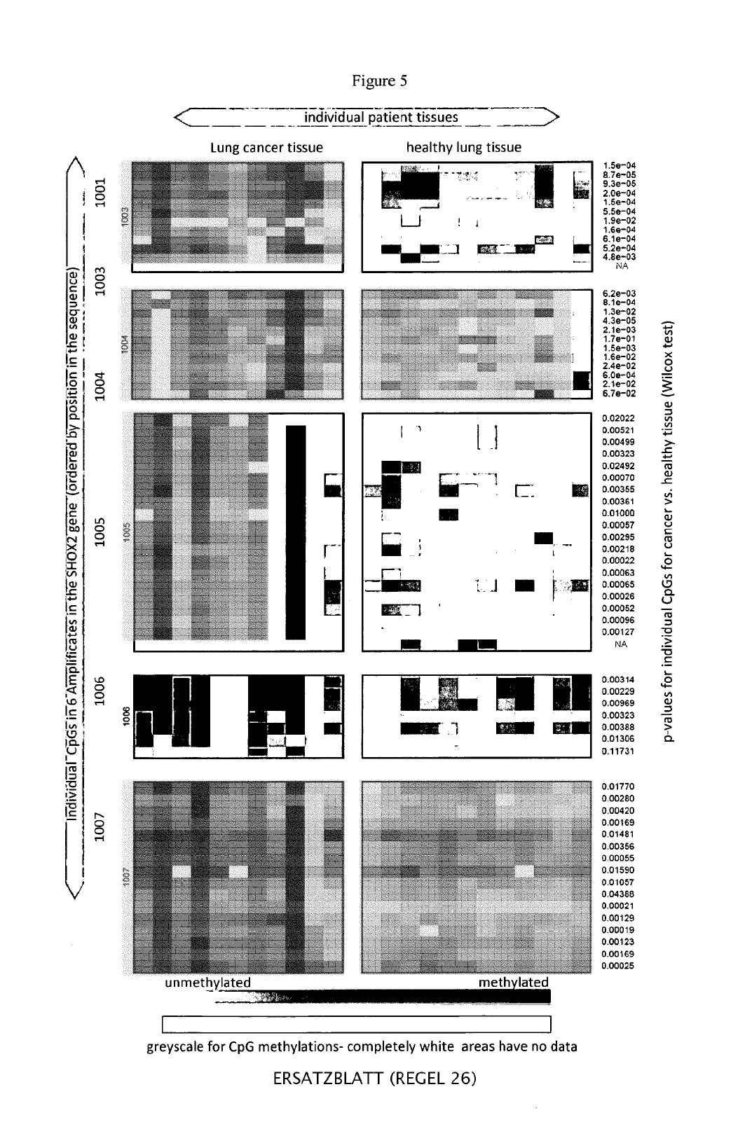

The sequencing data provides quantitative DNA methylation data on the CpG level. Within the amplificates shown in Table 3 and in FIG. 4, it is clearly seen that the differential methylation between lung cancer vs. healthy lung is a biological information that is not restricted to the location of the assessed regions of Example 1. In fact, the diagnostic value is seen in a long range of co-methylated area (see FIG. 5). All six amplificates clearly show differential methylation for lung cancer (from 11 lung cancer patients) vs. healthy lung tissue (from 12 different healthy individuals) over their whole range. P-values for individual CpGs (Wilcox test) clearly indicate that the differences are significant over the whole investigated range. It is expected that the methylation profile is extended beyond the assessed regions. SHOX2 DNA released from a lung cancer is expected to be identifiable due to its methylation status far beyond the assays in Example 1. This shows the phenomenon of co-methylation in SHOX2 and that it needs to be expected that CpG sites within the whole genomic sequence of SHOX2 can be used in accordance with the invention.

1. A method for monitoring a cancer under treatment, comprising determining the amount of hypermethylated shox2 genomic DNA in a first and one or more further test samples of a cancer patient taken subsequently.

2. A method for predicting the effect of a cancer treatment, comprising determining the amount of hypermethylated shox2 genomic DNA in a first and one or more further test samples of a cancer patient taken subsequently, wherein a change in or a stagnation of the amount of hypermethylated shox2 genomic DNA indicates the effect of the treatment.

3. A method for identifying a patient as a responder to a cancer treatment, comprising determining the amount of hypermethylated shox2 genomic DNA in a first and one or more further test samples of a cancer patient taken subsequently, wherein a substantial decrease in the amount of hypermethylated shox2 genomic DNA indicates a response to the treatment.

4. A method for identifying a patient as a non-responder to a cancer treatment, comprising determining the amount of hypermethylated shox2 genomic DNA in a first and one or more further test samples of a cancer patient taken subsequently, wherein an increase, a stagnation or a non-substantial decrease in the amount of hypermethylated shox2 genomic DNA indicates a non-response to the treatment.

5. A method for treating cancer, comprising the steps:
(i) determining the amount of hypermethylated shox2 genomic DNA in a first test sample of a cancer patient;
(ii) starting treatment of said patient with a first treatment regimen comprising one or more anti-cancer agents or therapies,
(iii) determining the amount of hypermethylated shox2 genomic DNA in one or more subsequently taken further test samples of said patient;
(iv) optionally repeating steps (ii) and (iii) one or more times;
(v) continuing treating the patient with the first treatment regimen if there is a substantial decrease in the amount of hypermethylated shox2 genomic DNA, or amending the treatment or terminating treating the patient with the first treatment regimen and treating the patient instead with a second treatment regimen comprising one or more anti-cancer agents or therapies not comprised in the first treatment regimen if there is an increase, a stagnation or a non-substantial decrease in the amount of hypermethylated shox2 genomic DNA.

6. The method of claim 1, wherein the cancer is selected from the group consisting of Adrenal Cancer, Anal Cancer, Bile Duct Cancer, Bladder Cancer, Bone Cancer, Brain/CNS Tumors, Breast Cancer, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Liver Cancer, Lung Cancer, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

7. The method of claim 6, wherein said cancer is lung cancer.

8. The method of claim 7, wherein said lung cancer is an advanced stage lung cancer.

9. The method of claim 1, wherein the test sample is derived from a body liquid.

10. The method of claim 9, wherein the test sample is blood plasma or blood serum.

11. The method of claim 1, wherein the treatment is a first line treatment.

12. The method of claim 1, wherein the first test sample is taken before the start of the treatment.

13. The method of claim 1, wherein the amount of hypermethylated shox2 genomic DNA in the sample is determined by a process selected from the group consisting of COBRA, restriction ligation-mediated PCR, Ms-SNuPE, ion-pair reverse-phase high performance liquid chromatography, denaturing high performance liquid chromatography, any bisulfite sequencing method, e.g. direct bisulfite sequencing with Sanger method or NexGen sequencing, or any pyrosequencing method, DNA sequencing methods that can per se distinguish between methylated and unmethylated cytosines, MALDI-TOF, QM™ and real-time PCR, preferably MethyLight™ or HeavyMethyl™ or a combination thereof.

14. The method of claim 1, wherein determining the amount of hypermethylated shox2 genomic DNA comprises a step of converting, in the genomic DNA, cytosine unmethylated in the 5-position to uracil or another base that does not hybridize to guanine.

15. The method of claim 14, wherein determining the amount of hypermethylated shox2 genomic DNA comprises a further step of amplifying at least a fragment of shox2 genomic DNA in a methylation dependent manner.

16. The method of claim 1, wherein determining the amount of hypermethylated shox2 genomic DNA comprises normalizing for the amount of total DNA in the sample.

17. The method of claim 16, wherein normalizing for the amount of total DNA in the test sample preferably comprises normalizing for the amount of a reference site in the genome.

18. The method of claim 17, wherein the reference site is a housekeeping gene or in proximity to a housekeeping gene.

19. The method of claim 16, wherein determining the amount of hypermethylated shox2 genomic DNA further comprises normalizing for the amount of shox2 hypermethylation of a fully methylated reference sample.

20. The method of claim 19, wherein the amount of hypermethylated shox2 genomic DNA is expressed as a PMR (percentage of methylated reference) value.

21. The method of claim 20, wherein the PMR value derived from the first test sample must be at least 1%.

22. The method of claim 1, wherein the amount of hypermethylated shox2 genomic DNA is determined in the first and one or more further test samples before a change in tumor size or in the amount of tumor cells (a) is determined, (b) would be determined, or (c) can be determined by conventional re-staging, respectively.


SEQUENCE LISTING (36 sequences)

SEQ ID NO: 1 - DNA, 10550 bp, Homo sapiens
ggaaagactatcccggatattttaatagtaacaggaacaaagatgactcgaaccccatcaaatgaaacgatattttcattcaattgatcgcaaagtgtcttcaattaataacttggcacttatttaaaaggtttaacagaattggaggcgacacattttgtactgacaacacgtcatgagataacatcttttgtttaaaataacttaatacactagaaaaaaatatgtcaaatataaataattttgttttcatggagaacaaatagttacaaagctcaaccgcataccaaagttcagttagaagagcttttcctctttcttgtaaattagaaggtaaaacaaaaacaaaaacaaaaccccacaaacttaggcttgctagcgaattgggatacagactggcttagtccaaggtatcccagtttaaatagctacatgaattgaaacaaacaaccaaaagaaaaaaaaaaacaaaaaagaaacaaacaaacaaaaaaaaaaggttacttgaatagatacacctttgtgagataaaatgcaaatgttgaaagtcagtccacacattatagtcaaaataaactttgttcgtgtgtatcaacatatcttacacaaacctagtgcctgtatctagctgggtgttatttaaggtgaatttgacgggatagagagggggaaataaaccactgtcttcaaataggacttcaggaaaccaacaagaaggaacacaagaaaaggggaatggtgggaactaatactgattgagcgcctactgtgtctggcacagcgaggcgctgtaccttcattatttcatttggtggtcttgttttctttttcctttccccacatattctgcccacctctgtcttcttaggacaatgagtgtaattttttccctttctgacttttttcttttgttccccaggatcaaagacagggcaaatatatatatatatatatatatatatatatatatatatatatggcaaatatatgatatatatatatggatatatatatatcaatttccagatacttttggtattgtttttcatagtgaagatgaaaatggagtgagtgcatgggagataagggggtgggaggagacacaccatcaatggaatacaagtaacatgcaaaccggaatttactgtcagcagtccagacagttttgccataataatttctcagagaaatcattgtctgtggcatttttttgtgttaatattatttttctctctcctcgttttaatattttctcttttcttgctcttccatacatggtgggtttaaactcaaagcatctgattcaacgtaaaaggaggcccgcctctctcatcaaatttctcgtgttaacgtgaacttcgcagaaaaaggtcagtttcaaaacctgtgaacttcccagctgcgcacttttttcatcttagttttttaaataagtatacctcttctttctgctttgccttcttagcgcatttttaaaaacacacatttaagttggtaactccccccagtctctcgagaatcgtctgggttgtttttccatatttttaaatgtataaaatactttaattttaaaatttgtttttgctccttggttctccaatttcagcttctgccaaatagcagttaagaaataagttcccttcctctttttctctcccgtttgtctttcgatttttttgtttgctcattttttcattgttaacaagatttttttttctatgcaagagtccatcgttgcagctttgcggtgagccaaactccgcggttccagcactcccctgtccagtctctctccagactcccccaaacccgctcctacaaaacccaattctaggccctcgagtaggaaaacgggcaggagccacggagcctgcgtgcctcgtgagatccctggtcctgcgtggagtctggctttccgagtccaagatgcgataggggacgagggatggtcagtgaggcgggaagagggccggctcccgaggtctcaaaggggtaacggagaagcagcggggcgcggagggcgtgcaggctgagtgccgcgggacaggcgcgacattggtgctggcgttggcgtcacagacccagggctgcggcgtgctttttggctttcagtctgagatcggcgatgctggagttcttgctggtggtcttggcggctgctgcggccgccactaccgaggcggcggaagccgaatccgcggccagcgtggcgagcggcagtccgaagggcggtgctgggaacatcatgtagggcgcgtgcgcggccaggtgcggatgcaggtggtggtgcgcgtgcgccacagcgctgtccagctgcagctgcgcctgaacctgaaaggacaagggcgtcacgttgcaatgactatcctagggtgacaacagaatagaaacagagcattaacggcgtccagcttggccagagagagagttgggttgtgtttgggcacggggagagggcatttggctgtgcatcttgttccgacagtgaccttctcagcctgttgctgaatctggggttccacatggatactccagtgtcccctgggtctccgaaactcgcttagggtaggcttatttccccggaccccgggaacccccttctgcaggaatgtggccgtctcccctgggaactgggctgaaatggtatattggaaagactgcgggtgaggacccttaccctcttctcttgatgctgccatgcatctcttaccccactaccaaaacaacggagtatcaacaagcaaactacgcaactttttaatgtcattcagggaagttactccatcttcaccacaataggcacaattgtgagtcggtacttctaacctttattgccataacaattacccagggatctcgttaaaattcagattctgattcgcaaggtctaggttggggcctgagattctgaatttctaacaatctcccagacggtgttgttgctgctggcccaaagattaccttttgagtagtaaaggtctccttttaaagtcacccctccctctttcttcagtcattctctcttgtttccaaagccaagcatcaggatgccagagtcctgtggcaccaagagagctaagatctccctaacaccgactctcaattcatggtcacacctatctgtctccccggtccaagtcactggagactgagagctaattcatgacatcacaggcatgccaagcaggggtcctggctgaggatagggaaggtcagacactggttgggaaaggatttcgccctcaggcttggaaagtggggagttcagagcactgggatttcccaggattaaatcattgctgcaggctgtccttttaaaatgtactcttaaaggtttctttatggctaagggattgcaaaggcagggcagccctgggaagaccaagacttctctctttaccagagatgaagctttgtttgcagaaacagatttaaaaacaaaagaacgaaaaaacaaaagtatgagctgggagtacttgtgtttatttttcttctgtcagagttatttaatgtgactaagaggtagaaaatacctagaaaagtatctagagggttgccttagaaataccttaggactaacctctgtattggatttgacaaaatcttaatcaaaaggcttttccttcctctctctctaagggcaatctttagtgtatttttaaaggaccagttactgcccccaggccccttcagggattcccatgaggacagaagggactaggcattcaggcctttaataccgtataggtttttaaagggtagtgggtccactatatcatctcaaaatgattctccaactatgcagacattgacatctgggctcagagacaggtgatgttcaacagttcaagcaaacattcaggtattttcatatttaaaaacagcttgaaactaggctgtacttgcctgctgaaatggcatccttaaagcacctacgttgacataaggtgcgactctacaagcttcaaactggctggcggcccctatgagaacacctgtaaaaagtacaagagaaaaataatgtgaggttaacgtttgtagcatttttcagggttccaaagggtacaagacagaacgaattcctttgaaaatggactattatctttctaaactgcatccaaaatttaaataaatgtaacttactcccaaactttaggactccattaacagcctttgaagattattaagtcaagaactattgtcatttttgaggttcctttttttctgtcattgtaatagtaatattcactacttctctctttgctcagactatcaaatgttccttgtataagaagcatacctttatggagttgattttcttgttttctacatttagctcttcgattttgaaaccaaacctataggttggagggggaaaaaaaataaaacctagatgttatgactaaaaatttttttaaattacaaaagtacaaagagaaaagagtcgtggtttagacaagaattgggagatacagcaagcaatctttgaattgtataaaacagcctcaaattccaaagttaaacatgtaaattaactacagaaatagaaagatagattttatagactttcacaagatagattactattagtcctgatttcatcttttcacataacagagtccccatggcctaaaactctgcttggtacctgataccaacatgataatggtttagatatttgcccagttaagacctgagaagaatgtgttcattaatagcctgtaatgttaattagcttcatcttaaagaaaaaccaaacaccagagcaaataatgactcctttgcggagaaggagttgtaagggttggggagaggtaattagtgtcataaaagcctcactacaaagaagtttagaatttctctgttctgctgtgcagcagagtgaaacacagtagcagaataattgtgcaccattatcatgataatctaatttgcttctttagaaaaagccactgtttgcagttaggatagtaggaaaacatctagaaaataactaaaatgtaaataaaagtatgaaacaacaataaagttcacttcttaagagaaaaaaagaagagcactcagcgaaaggagaatgaagtatatataaagcaagtaactcttaggtactttgttctcctggatgttacctgcttaaacttgacaacttttacaggcagtatgacagattttttaaaattcaaataaagcttgcttttgtattccactttcaataataaagcatattgattgactcacaaattaaagtttttaaaaccccattaaagtaaaaatactgcttcagagctttcattggtatgtttttgaaagcaagtgtcatagtcaatgtaaatcttcaaaaatttctctgctatttagtacaacagagggtggattttgtgccagggtggcatcctttgtgtaattcacatggcctgaggcttcttgcctttttttcccctagaagagttggaattaagatactaataatcataactgtaataattatcatcatgtttttaaggggttgctgagcctctggagatctcagtgcacagagcgaagcaggagaggcttaaaaaggaaggctctgctgctgatgctgtgatgaccaaggcagcctattattacttttcctcttcaacgtgactgctctgtttttttttttcctgatcctgttacaagatgtggggaagccccttaagaattgtgcatagttcgctacagatggtggtctctgtgcccagagaccgaaagagtatctgttacagcgatactttgtaacagctgcagatgtgcgggcctaaaactgctccatgtgttcaaaccaataataactatcattttattctattttattatctgagcggaaatacaatctgaaacatttaaatttgttccccctaaagctagaaggatatgtgtatgtgaatgtacacaatttgctcctttaaatgcccccaaagcctatttttgctattacatttgcctccacaaccaataaagaaaatctgtttcagtcctaacttcattgggtatggcacgtgctggagggcaggctttggaaatataaatactaatatatgtggtttgttttaaaatctaatgtatttagggtcagtttcgttgccttggttaaatgaatgaacaaagagtgaccaaaatgcgtttatttctccgctatttcacacacagtttgagatcgaccgctaacagcagactgtaaatttctgcacttaacagttactaattttctttatagtctgcacatctataaacctaattgggatatccgcttgacaaacctagaccctgcagagcagactttcaaggaaatccaactccagagaacaaaatactctttggaacttttaaggcatccagagataaaacgttttctgtgggatttttttttaagccttataaaagtaggcccattaggattaaaccacattaaagccagtgcgaggcaaggggggtgtggtttatagaaactctgggagaaagcccagcacagctttcttcttctgggaacatatgttaggatggaaaaaaaaaaaaggaggggagtgctataggactaagatctactttggaaaataagacctctagtaaagatatctctttccttctcatcttacaccagacactagaagcaccacctcccgagtgtgtcccccaagcctctttccaaccccaggctctctctgctccctgggccctcgacctcctggcagctgggtacctgcactcgggcctccgacaggcccagtcgctggctcagttcctctcgcatgaaggcgtcgggatagtgggtctcgtcaaaaagcctctccagctcattgagttgttccagggtgaaattggtccgacttcgcctctgcttgattttggtctggccttcgtcctccatccctttcgcatcctctttgcgatctttcagctccggggacactggagggggcaccccagcggggccacacgtgcatccacacgaacacacacacacacgcacacacacacggacgaaaacagcacagcaaatcctgttaccagatttgtcgcaaaaaagaatagagaatccttccccctcgtggaatcctggtccggcttctcaacccacccggaggaagaatatcccggagaagcgcaaaggtcaagtctgagcggccgcctggggcaaggtgggcagtcatccaccacctccttcacaaacgtacctcgactcaccgcctcactattcaccccccagtcaccggccttccccagcaaacaccaaatggtttccactcactttcgcactcacttgctccgcagcactcacagaaatcaaaggcgcagcgccaggcctctcacagagcaaagtctggacataagggcgcggcaaggccacccactggcctctcacaccggccattcccagaaggctggctcgttctcaggccagctctcaccaccgctggctctctgcctaccgcaaacttgctggtctaatttaggaacaattgggccgaaaggtatcagcgagagcaacagaccccggtgttgtgccgcacagggagccgcatccgcagacgcccctcgctgcccctgggctcgggccaaaccctgcataaggtcccctggacagccaggtaatctccgtcccgcctgcccgaccggggtcgcacgagcacaggcgcccacgccatgttggctgcccaaagggctcgccgcccaagccgggccagaaggcaggaggcggaaaaccagcctccggtggcgggcgaaagcaaccgctctttctgttctctcttcgccctccctcgtggaaacgcagactcgaccctaaacgcttaacccacagagatcaacaggttcaagcggaatattcgcgatcctcggtttctattggttgctcaaagccttttcatgcaaccagcagctcggatgtttaataaaatatgaatttttctgctctgggtttgcacacatccgttgccactttcctgtccttctcctccctttaaaaacaggagctcttctagtgtgagcatatacagccaccatatatatatccccctggaacctttatcctctgcgcccgaatcagagactccgggttttaagaagtcaaagctgagtttagatagtcgaggctgtttgcagttcttcccagcgcaggatccaggccagcatgggtgttaaaaatcatttccggaagagccggtctgcagagaaattaatacttgccctgagcttggaaatcaacaatcccaggaactgcgcccgccgggggttcagacctgaacctcgccgtcaggcgcgctggcgaacctgtagttcgggcctgtagttgtggactcatggccgggaggctgcactgggcaccttgacggtaactccaccactttacaaatttaggaagacctggtgctggtttggaggaagaagagtcgctagatctcggccctgccgggcattctagctacggtgatcttaaatattgccgtattttctagttctcagagttagttttgtggacatttgtcctttctttaggaaggaacattatagtaaacattatgggccaatttaatagcaaagtatttttactgatattgtattgaaatttaaatatgaaaacacacccttcaaattctgccaatgaggtgggaaggctttgtctattacatgtcatctacacagtggatgccactcaaagttttggggcttcgaggagatgtccaaatttcaagttgatccatagacttaataaaatctctatttttgtccccaaatttccaattactgatctaaacaggaacagaccgtgtcataaacaacttaattgcttgcttaaatatgtgattccacgcatgccacatattttcttaattggaaaaatatggcagtacaccagttactgcaggcaccaagtgccaaatcaattttattgttgaactcctttgaattggtatagaacgctctgccattgagataaagacgtttcctaacactgagctagcaccaaaggatttttgctctaagagaaccgagtactgggtgattgaaaacactaaaggaattgtgtaaatatagatccccacctcccccgccgccccccccccccgcccccaacacaccaagaaaccgaaacgcctcgcccgggagaagcagcgtagcctggacctcagctttcgttggcttccttctaccttgaaagagaatgggaagttttggcccctaagatccaaagattcaggggctctgcggagttcgaggggtcttggcggccccaaacacctaggcgaccggagggttaagctttccacctctgccccttctccctcccggcaaactctgcgctagaggctagctttagtcccgccgtctggttcagcaccccctcctgcagcccggccccgcgaacttccacgtccgcctgcgggccatccgggctgcgggcccatcctcccgctggtgaaaaatgcatttcggtggaatgggaacatctgggaagggcgcgcacaaaccccacacgcttccccccacgccacccccacaacacactaggaccccacccatgaccgagcataccaccggacccccacccttccactatcactctagctgacaaagctggggtccagggccctctccgtgtccctctcccctccccgctgggcctcggagtcctctcccgcccgagaggagtccggaaggcgggaaggggaaccgctgccgggggtcagtcaggtcgttaccctccgtcagtcgcgggctgcccggctccctgcttctctcggcggcgcccatgtccagctcccggacgggagagcgccctcctccagctcctccgcctgctcctcctcctcctacacctcctccgcctcctccgccgccgcctccgcctcctccgccgccgcctccgccggccgcccggactgccgggctgctgcggtcgtcgcggcccgcctcggtgcagccggtcggctccttggccccgcgcagcggcccgctctccagcacctcccggtacgtgatcgcctccttcttctccttcactttctggtcaaaagacttggagacgaacgccgtaagttcttccatcgccgccgcacgtcagcccggcgctcaacctctgccagcagagccccgctctttttttccttcttctttttttactgctccagcccccccaataataacacatcaatgggacaggaggtgggggaggagagggaggaggagaaagaagaagaaaaagaggagaagaaagaagaaagaggaggagaagtagaaggagaaaaagaagtaagaagaggaggaggaggaagaagaggaagagggggagaagggtgaagaggagagggaggaggaggaggagaagagaaggggcgggggctgccggagggaaatgggaactgatgcttccctgccggagcgcgcttgttcaatgttaagatctttgacaattcttcctgcggagagttcagatctgatagcgacgctgcgcttgaagtcccactatttatacttcctaacggcggcctcttgttttgagtaaattacctcccctcgcaactcgg

SEQ ID NO: 2 - DNA, 10550 bp, Artificial Sequence
  SEQ ID NO 1 converted C to T
ggaaagattatttcggatattttaatagtaataggaataaagatgattcgaattttattaaatgaaacgatatttttatttaattgatcgtaaagtgtttttaattaataatttggtatttatttaaaaggtttaatagaattggaggcgatatattttgtattgataatacgttatgagataatattttttgtttaaaataatttaatatattagaaaaaaatatgttaaatataaataattttgtttttatggagaataaatagttataaagtttaatcgtatattaaagtttagttagaagagtttttttttttttttgtaaattagaaggtaaaataaaaataaaaataaaattttataaatttaggtttgttagcgaattgggatatagattggtttagtttaaggtattttagtttaaatagttatatgaattgaaataaataattaaaagaaaaaaaaaaataaaaaagaaataaataaataaaaaaaaaaggttatttgaatagatatatttttgtgagataaaatgtaaatgttgaaagttagtttatatattatagttaaaataaattttgttcgtgtgtattaatatattttatataaatttagtgtttgtatttagttgggtgttatttaaggtgaatttgacgggatagagagggggaaataaattattgtttttaaataggattttaggaaattaataagaaggaatataagaaaaggggaatggtgggaattaatattgattgagcgtttattgtgtttggtatagcgaggcgttgtatttttattattttatttggtggttttgtttttttttttttttttttatatattttgtttatttttgtttttttaggataatgagtgtaattttttttttttttgattttttttttttgttttttaggattaaagatagggtaaatatatatatatatatatatatatatatatatatatatatatggtaaatatatgatatatatatatggatatatatatattaatttttagatatttttggtattgttttttatagtgaagatgaaaatggagtgagtgtatgggagataagggggtgggaggagatatattattaatggaatataagtaatatgtaaatcggaatttattgttagtagtttagatagttttgttataataattttttagagaaattattgtttgtggtatttttttgtgttaatattattttttttttttttcgttttaatattttttttttttttgtttttttatatatggtgggtttaaatttaaagtatttgatttaacgtaaaaggaggttcgttttttttattaaatttttcgtgttaacgtgaatttcgtagaaaaaggttagttttaaaatttgtgaattttttagttgcgtattttttttattttagttttttaaataagtatattttttttttttgttttgtttttttagcgtatttttaaaaatatatatttaagttggtaattttttttagtttttcgagaatcgtttgggttgtttttttatatttttaaatgtataaaatattttaattttaaaatttgtttttgttttttggttttttaattttagtttttgttaaatagtagttaagaaataagttttttttttttttttttttttcgtttgtttttcgatttttttgtttgtttatttttttattgttaataagatttttttttttatgtaagagtttatcgttgtagttttgcggtgagttaaatttcgcggttttagtatttttttgtttagtttttttttagatttttttaaattcgtttttataaaatttaattttaggttttcgagtaggaaaacgggtaggagttacggagtttgcgtgtttcgtgagatttttggttttgcgtggagtttggtttttcgagtttaagatgcgataggggacgagggatggttagtgaggcgggaagagggtcggttttcgaggttttaaaggggtaacggagaagtagcggggcgcggagggcgtgtaggttgagtgtcgcgggataggcgcgatattggtgttggcgttggcgttatagatttagggttgcggcgtgttttttggtttttagtttgagatcggcgatgttggagtttttgttggtggttttggcggttgttgcggtcgttattatcgaggcggcggaagtcgaattcgcggttagcgtggcgagcggtagttcgaagggcggtgttgggaatattatgtagggcgcgtgcgcggttaggtgcggatgtaggtggtggtgcgcgtgcgttatagcgttgtttagttgtagttgcgtttgaatttgaaaggataagggcgttacgttgtaatgattattttagggtgataatagaatagaaatagagtattaacggcgtttagtttggttagagagagagttgggttgtgtttgggtacggggagagggtatttggttgtgtattttgtttcgatagtgatttttttagtttgttgttgaatttggggttttatatggatattttagtgttttttgggttttcgaaattcgtttagggtaggtttattttttcggatttcgggaatttttttttgtaggaatgtggtcgttttttttgggaattgggttgaaatggtatattggaaagattgcgggtgaggatttttattttttttttttgatgttgttatgtattttttattttattattaaaataacggagtattaataagtaaattacgtaattttttaatgttatttagggaagttattttatttttattataataggtataattgtgagtcggtatttttaatttttattgttataataattatttagggatttcgttaaaatttagattttgattcgtaaggtttaggttggggtttgagattttgaatttttaataattttttagacggtgttgttgttgttggtttaaagattattttttgagtagtaaaggtttttttttaaagttatttttttttttttttttagttatttttttttgtttttaaagttaagtattaggatgttagagttttgtggtattaagagagttaagatttttttaatatcgatttttaatttatggttatatttatttgttttttcggtttaagttattggagattgagagttaatttatgatattataggtatgttaagtaggggttttggttgaggatagggaaggttagatattggttgggaaaggatttcgtttttaggtttggaaagtggggagtttagagtattgggattttttaggattaaattattgttgtaggttgtttttttaaaatgtatttttaaaggtttttttatggttaagggattgtaaaggtagggtagttttgggaagattaagatttttttttttattagagatgaagttttgtttgtagaaatagatttaaaaataaaagaacgaaaaaataaaagtatgagttgggagtatttgtgtttattttttttttgttagagttatttaatgtgattaagaggtagaaaatatttagaaaagtatttagagggttgttttagaaatattttaggattaatttttgtattggatttgataaaattttaattaaaaggttttttttttttttttttttaagggtaatttttagtgtatttttaaaggattagttattgtttttaggtttttttagggatttttatgaggatagaagggattaggtatttaggtttttaatatcgtataggtttttaaagggtagtgggtttattatattattttaaaatgattttttaattatgtagatattgatatttgggtttagagataggtgatgtttaatagtttaagtaaatatttaggtatttttatatttaaaaatagtttgaaattaggttgtatttgtttgttgaaatggtatttttaaagtatttacgttgatataaggtgcgattttataagttttaaattggttggcggtttttatgagaatatttgtaaaaagtataagagaaaaataatgtgaggttaacgtttgtagtattttttagggttttaaagggtataagatagaacgaatttttttgaaaatggattattatttttttaaattgtatttaaaatttaaataaatgtaatttatttttaaattttaggattttattaatagtttttgaagattattaagttaagaattattgttatttttgaggtttttttttttttgttattgtaatagtaatatttattatttttttttttgtttagattattaaatgttttttgtataagaagtatatttttatggagttgatttttttgttttttatatttagtttttcgattttgaaattaaatttataggttggagggggaaaaaaaataaaatttagatgttatgattaaaaatttttttaaattataaaagtataaagagaaaagagtcgtggtttagataagaattgggagatatagtaagtaatttttgaattgtataaaatagttttaaattttaaagttaaatatgtaaattaattatagaaatagaaagatagattttatagatttttataagatagattattattagttttgattttatttttttatataatagagtttttatggtttaaaattttgtttggtatttgatattaatatgataatggtttagatatttgtttagttaagatttgagaagaatgtgtttattaatagtttgtaatgttaattagttttattttaaagaaaaattaaatattagagtaaataatgatttttttgcggagaaggagttgtaagggttggggagaggtaattagtgttataaaagttttattataaagaagtttagaatttttttgttttgttgtgtagtagagtgaaatatagtagtagaataattgtgtattattattatgataatttaatttgtttttttagaaaaagttattgtttgtagttaggatagtaggaaaatatttagaaaataattaaaatgtaaataaaagtatgaaataataataaagtttattttttaagagaaaaaaagaagagtatttagcgaaaggagaatgaagtatatataaagtaagtaatttttaggtattttgtttttttggatgttatttgtttaaatttgataatttttataggtagtatgatagattttttaaaatttaaataaagtttgtttttgtattttatttttaataataaagtatattgattgatttataaattaaagtttttaaaattttattaaagtaaaaatattgttttagagtttttattggtatgtttttgaaagtaagtgttatagttaatgtaaatttttaaaaatttttttgttatttagtataatagagggtggattttgtgttagggtggtattttttgtgtaatttatatggtttgaggttttttgttttttttttttttagaagagttggaattaagatattaataattataattgtaataattattattatgtttttaaggggttgttgagtttttggagattttagtgtatagagcgaagtaggagaggtttaaaaaggaaggttttgttgttgatgttgtgatgattaaggtagtttattattattttttttttttaacgtgattgttttgttttttttttttttgattttgttataagatgtggggaagttttttaagaattgtgtatagttcgttatagatggtggtttttgtgtttagagatcgaaagagtatttgttatagcgatattttgtaatagttgtagatgtgcgggtttaaaattgttttatgtgtttaaattaataataattattattttattttattttattatttgagcggaaatataatttgaaatatttaaatttgttttttttaaagttagaaggatatgtgtatgtgaatgtatataatttgtttttttaaatgtttttaaagtttatttttgttattatatttgtttttataattaataaagaaaatttgttttagttttaattttattgggtatggtacgtgttggagggtaggttttggaaatataaatattaatatatgtggtttgttttaaaatttaatgtatttagggttagtttcgttgttttggttaaatgaatgaataaagagtgattaaaatgcgtttattttttcgttattttatatatagtttgagatcgatcgttaatagtagattgtaaatttttgtatttaatagttattaattttttttatagtttgtatatttataaatttaattgggatattcgtttgataaatttagattttgtagagtagatttttaaggaaatttaattttagagaataaaatattttttggaatttttaaggtatttagagataaaacgttttttgtgggatttttttttaagttttataaaagtaggtttattaggattaaattatattaaagttagtgcgaggtaaggggggtgtggtttatagaaattttgggagaaagtttagtatagttttttttttttgggaatatatgttaggatggaaaaaaaaaaaaggaggggagtgttataggattaagatttattttggaaaataagatttttagtaaagatattttttttttttttattttatattagatattagaagtattatttttcgagtgtgttttttaagtttttttttaattttaggttttttttgttttttgggttttcgattttttggtagttgggtatttgtattcgggttttcgataggtttagtcgttggtttagtttttttcgtatgaaggcgtcgggatagtgggtttcgttaaaaagtttttttagtttattgagttgttttagggtgaaattggttcgatttcgtttttgtttgattttggtttggttttcgttttttatttttttcgtattttttttgcgattttttagtttcggggatattggagggggtattttagcggggttatacgtgtatttatacgaatatatatatatacgtatatatatacggacgaaaatagtatagtaaattttgttattagatttgtcgtaaaaaagaatagagaatttttttttttcgtggaattttggttcggttttttaatttattcggaggaagaatatttcggagaagcgtaaaggttaagtttgagcggtcgtttggggtaaggtgggtagttatttattattttttttataaacgtatttcgatttatcgttttattatttattttttagttatcggttttttttagtaaatattaaatggtttttatttattttcgtatttatttgtttcgtagtatttatagaaattaaaggcgtagcgttaggttttttatagagtaaagtttggatataagggcgcggtaaggttatttattggttttttatatcggttatttttagaaggttggttcgtttttaggttagtttttattatcgttggttttttgtttatcgtaaatttgttggtttaatttaggaataattgggtcgaaaggtattagcgagagtaatagatttcggtgttgtgtcgtatagggagtcgtattcgtagacgtttttcgttgtttttgggttcgggttaaattttgtataaggttttttggatagttaggtaattttcgtttcgtttgttcgatcggggtcgtacgagtataggcgtttacgttatgttggttgtttaaagggttcgtcgtttaagtcgggttagaaggtaggaggcggaaaattagttttcggtggcgggcgaaagtaatcgttttttttgtttttttttcgttttttttcgtggaaacgtagattcgattttaaacgtttaatttatagagattaataggtttaagcggaatattcgcgattttcggtttttattggttgtttaaagtttttttatgtaattagtagttcggatgtttaataaaatatgaatttttttgttttgggtttgtatatattcgttgttatttttttgttttttttttttttttaaaaataggagtttttttagtgtgagtatatatagttattatatatatatttttttggaatttttattttttgcgttcgaattagagatttcgggttttaagaagttaaagttgagtttagatagtcgaggttgtttgtagttttttttagcgtaggatttaggttagtatgggtgttaaaaattattttcggaagagtcggtttgtagagaaattaatatttgttttgagtttggaaattaataattttaggaattgcgttcgtcgggggtttagatttgaatttcgtcgttaggcgcgttggcgaatttgtagttcgggtttgtagttgtggatttatggtcgggaggttgtattgggtattttgacggtaattttattattttataaatttaggaagatttggtgttggtttggaggaagaagagtcgttagatttcggttttgtcgggtattttagttacggtgattttaaatattgtcgtattttttagtttttagagttagttttgtggatatttgttttttttttaggaaggaatattatagtaaatattatgggttaatttaatagtaaagtatttttattgatattgtattgaaatttaaatatgaaaatatattttttaaattttgttaatgaggtgggaaggttttgtttattatatgttatttatatagtggatgttatttaaagttttggggtttcgaggagatgtttaaattttaagttgatttatagatttaataaaatttttatttttgtttttaaatttttaattattgatttaaataggaatagatcgtgttataaataatttaattgtttgtttaaatatgtgattttacgtatgttatatatttttttaattggaaaaatatggtagtatattagttattgtaggtattaagtgttaaattaattttattgttgaatttttttgaattggtatagaacgttttgttattgagataaagacgttttttaatattgagttagtattaaaggatttttgttttaagagaatcgagtattgggtgattgaaaatattaaaggaattgtgtaaatatagatttttatttttttcgtcgtttttttttttcgtttttaatatattaagaaatcgaaacgtttcgttcgggagaagtagcgtagtttggattttagttttcgttggtttttttttattttgaaagagaatgggaagttttggtttttaagatttaaagatttaggggttttgcggagttcgaggggttttggcggttttaaatatttaggcgatcggagggttaagttttttatttttgttttttttttttttcggtaaattttgcgttagaggttagttttagtttcgtcgtttggtttagtatttttttttgtagttcggtttcgcgaatttttacgttcgtttgcgggttattcgggttgcgggtttattttttcgttggtgaaaaatgtatttcggtggaatgggaatatttgggaagggcgcgtataaattttatacgtttttttttacgttatttttataatatattaggattttatttatgatcgagtatattatcggatttttatttttttattattattttagttgataaagttggggtttagggtttttttcgtgtttttttttttttttcgttgggtttcggagttttttttcgttcgagaggagttcggaaggcgggaaggggaatcgttgtcgggggttagttaggtcgttatttttcgttagtcgcgggttgttcggttttttgtttttttcggcggcgtttatgtttagttttcggacgggagagcgtttttttttagttttttcgtttgttttttttttttttatatttttttcgtttttttcgtcgtcgttttcgtttttttcgtcgtcgttttcgtcggtcgttcggattgtcgggttgttgcggtcgtcgcggttcgtttcggtgtagtcggtcggttttttggtttcgcgtagcggttcgttttttagtatttttcggtacgtgatcgtttttttttttttttttattttttggttaaaagatttggagacgaacgtcgtaagtttttttatcgtcgtcgtacgttagttcggcgtttaatttttgttagtagagtttcgtttttttttttttttttttttttttattgttttagtttttttaataataatatattaatgggataggaggtgggggaggagagggaggaggagaaagaagaagaaaaagaggagaagaaagaagaaagaggaggagaagtagaaggagaaaaagaagtaagaagaggaggaggaggaagaagaggaagagggggagaagggtgaagaggagagggaggaggaggaggagaagagaaggggcgggggttgtcggagggaaatgggaattgatgtttttttgtcggagcgcgtttgtttaatgttaagatttttgataattttttttgcggagagtttagatttgatagcgacgttgcgtttgaagttttattatttatattttttaacggcggttttttgttttgagtaaattattttttttcgtaattcgg

SEQ ID NO: 3 - DNA, 10550 bp, Artificial Sequence
  SEQ ID NO 1 converted C to T, reverse complement
ccgaattacgaaaaaaaataatttactcaaaacaaaaaaccgccgttaaaaaatataaataataaaacttcaaacgcaacgtcgctatcaaatctaaactctccgcaaaaaaaattatcaaaaatcttaacattaaacaaacgcgctccgacaaaaaaacatcaattcccatttccctccgacaacccccgccccttctcttctcctcctcctcctccctctcctcttcacccttctccccctcttcctcttcttcctcctcctcctcttcttacttctttttctccttctacttctcctcctctttcttctttcttctcctctttttcttcttctttctcctcctccctctcctcccccacctcctatcccattaatatattattattaaaaaaactaaaacaataaaaaaaaaaaaaaaaaaaaaaaaacgaaactctactaacaaaaattaaacgccgaactaacgtacgacgacgataaaaaaacttacgacgttcgtctccaaatcttttaaccaaaaaataaaaaaaaaaaaaaaaacgatcacgtaccgaaaaatactaaaaaacgaaccgctacgcgaaaccaaaaaaccgaccgactacaccgaaacgaaccgcgacgaccgcaacaacccgacaatccgaacgaccgacgaaaacgacgacgaaaaaaacgaaaacgacgacgaaaaaaacgaaaaaaatataaaaaaaaaaaaaacaaacgaaaaaactaaaaaaaaacgctctcccgtccgaaaactaaacataaacgccgccgaaaaaaacaaaaaaccgaacaacccgcgactaacgaaaaataacgacctaactaacccccgacaacgattccccttcccgccttccgaactcctctcgaacgaaaaaaaactccgaaacccaacgaaaaaaaaaaaaaaacacgaaaaaaaccctaaaccccaactttatcaactaaaataataataaaaaaataaaaatccgataatatactcgatcataaataaaatcctaatatattataaaaataacgtaaaaaaaaacgtataaaatttatacgcgcccttcccaaatattcccattccaccgaaatacatttttcaccaacgaaaaaataaacccgcaacccgaataacccgcaaacgaacgtaaaaattcgcgaaaccgaactacaaaaaaaaatactaaaccaaacgacgaaactaaaactaacctctaacgcaaaatttaccgaaaaaaaaaaaaaacaaaaataaaaaacttaaccctccgatcgcctaaatatttaaaaccgccaaaacccctcgaactccgcaaaacccctaaatctttaaatcttaaaaaccaaaacttcccattctctttcaaaataaaaaaaaaccaacgaaaactaaaatccaaactacgctacttctcccgaacgaaacgtttcgatttcttaatatattaaaaacgaaaaaaaaaaacgacgaaaaaaataaaaatctatatttacacaattcctttaatattttcaatcacccaatactcgattctcttaaaacaaaaatcctttaatactaactcaatattaaaaaacgtctttatctcaataacaaaacgttctataccaattcaaaaaaattcaacaataaaattaatttaacacttaatacctacaataactaatatactaccatatttttccaattaaaaaaatatataacatacgtaaaatcacatatttaaacaaacaattaaattatttataacacgatctattcctatttaaatcaataattaaaaatttaaaaacaaaaataaaaattttattaaatctataaatcaacttaaaatttaaacatctcctcgaaaccccaaaactttaaataacatccactatataaataacatataataaacaaaaccttcccacctcattaacaaaatttaaaaaatatattttcatatttaaatttcaatacaatatcaataaaaatactttactattaaattaacccataatatttactataatattccttcctaaaaaaaaaacaaatatccacaaaactaactctaaaaactaaaaaatacgacaatatttaaaatcaccgtaactaaaatacccgacaaaaccgaaatctaacgactcttcttcctccaaaccaacaccaaatcttcctaaatttataaaataataaaattaccgtcaaaatacccaatacaacctcccgaccataaatccacaactacaaacccgaactacaaattcgccaacgcgcctaacgacgaaattcaaatctaaacccccgacgaacgcaattcctaaaattattaatttccaaactcaaaacaaatattaatttctctacaaaccgactcttccgaaaataatttttaacacccatactaacctaaatcctacgctaaaaaaaactacaaacaacctcgactatctaaactcaactttaacttcttaaaacccgaaatctctaattcgaacgcaaaaaataaaaattccaaaaaaatatatatataataactatatatactcacactaaaaaaactcctatttttaaaaaaaaaaaaaaaacaaaaaaataacaacgaatatatacaaacccaaaacaaaaaaattcatattttattaaacatccgaactactaattacataaaaaaactttaaacaaccaataaaaaccgaaaatcgcgaatattccgcttaaacctattaatctctataaattaaacgtttaaaatcgaatctacgtttccacgaaaaaaaacgaaaaaaaaacaaaaaaaacgattactttcgcccgccaccgaaaactaattttccgcctcctaccttctaacccgacttaaacgacgaaccctttaaacaaccaacataacgtaaacgcctatactcgtacgaccccgatcgaacaaacgaaacgaaaattacctaactatccaaaaaaccttatacaaaatttaacccgaacccaaaaacaacgaaaaacgtctacgaatacgactccctatacgacacaacaccgaaatctattactctcgctaatacctttcgacccaattattcctaaattaaaccaacaaatttacgataaacaaaaaaccaacgataataaaaactaacctaaaaacgaaccaaccttctaaaaataaccgatataaaaaaccaataaataaccttaccgcgcccttatatccaaactttactctataaaaaacctaacgctacgcctttaatttctataaatactacgaaacaaataaatacgaaaataaataaaaaccatttaatatttactaaaaaaaaccgataactaaaaaataaataataaaacgataaatcgaaatacgtttataaaaaaaataataaataactacccaccttaccccaaacgaccgctcaaacttaacctttacgcttctccgaaatattcttcctccgaataaattaaaaaaccgaaccaaaattccacgaaaaaaaaaaattctctattcttttttacgacaaatctaataacaaaatttactatactattttcgtccgtatatatatacgtatatatatatattcgtataaatacacgtataaccccgctaaaataccccctccaatatccccgaaactaaaaaatcgcaaaaaaaatacgaaaaaaataaaaaacgaaaaccaaaccaaaatcaaacaaaaacgaaatcgaaccaatttcaccctaaaacaactcaataaactaaaaaaactttttaacgaaacccactatcccgacgccttcatacgaaaaaaactaaaccaacgactaaacctatcgaaaacccgaatacaaatacccaactaccaaaaaatcgaaaacccaaaaaacaaaaaaaacctaaaattaaaaaaaaacttaaaaaacacactcgaaaaataatacttctaatatctaatataaaataaaaaaaaaaaaaatatctttactaaaaatcttattttccaaaataaatcttaatcctataacactcccctccttttttttttttccatcctaacatatattcccaaaaaaaaaaaactatactaaactttctcccaaaatttctataaaccacaccccccttacctcgcactaactttaatataatttaatcctaataaacctacttttataaaacttaaaaaaaaatcccacaaaaaacgttttatctctaaataccttaaaaattccaaaaaatattttattctctaaaattaaatttccttaaaaatctactctacaaaatctaaatttatcaaacgaatatcccaattaaatttataaatatacaaactataaaaaaaattaataactattaaatacaaaaatttacaatctactattaacgatcgatctcaaactatatataaaataacgaaaaaataaacgcattttaatcactctttattcattcatttaaccaaaacaacgaaactaaccctaaatacattaaattttaaaacaaaccacatatattaatatttatatttccaaaacctaccctccaacacgtaccatacccaataaaattaaaactaaaacaaattttctttattaattataaaaacaaatataataacaaaaataaactttaaaaacatttaaaaaaacaaattatatacattcacatacacatatccttctaactttaaaaaaaacaaatttaaatatttcaaattatatttccgctcaaataataaaataaaataaaataataattattattaatttaaacacataaaacaattttaaacccgcacatctacaactattacaaaatatcgctataacaaatactctttcgatctctaaacacaaaaaccaccatctataacgaactatacacaattcttaaaaaacttccccacatcttataacaaaatcaaaaaaaaaaaaaacaaaacaatcacgttaaaaaaaaaaaataataataaactaccttaatcatcacaacatcaacaacaaaaccttcctttttaaacctctcctacttcgctctatacactaaaatctccaaaaactcaacaaccccttaaaaacataataataattattacaattataattattaatatcttaattccaactcttctaaaaaaaaaaaaaacaaaaaacctcaaaccatataaattacacaaaaaataccaccctaacacaaaatccaccctctattatactaaataacaaaaaaatttttaaaaatttacattaactataacacttactttcaaaaacataccaataaaaactctaaaacaatatttttactttaataaaattttaaaaactttaatttataaatcaatcaatatactttattattaaaaataaaatacaaaaacaaactttatttaaattttaaaaaatctatcatactacctataaaaattatcaaatttaaacaaataacatccaaaaaaacaaaatacctaaaaattacttactttatatatacttcattctcctttcgctaaatactcttctttttttctcttaaaaaataaactttattattatttcatacttttatttacattttaattattttctaaatattttcctactatcctaactacaaacaataactttttctaaaaaaacaaattaaattatcataataataatacacaattattctactactatatttcactctactacacaacaaaacaaaaaaattctaaacttctttataataaaacttttataacactaattacctctccccaacccttacaactccttctccgcaaaaaaatcattatttactctaatatttaatttttctttaaaataaaactaattaacattacaaactattaataaacacattcttctcaaatcttaactaaacaaatatctaaaccattatcatattaatatcaaataccaaacaaaattttaaaccataaaaactctattatataaaaaaataaaatcaaaactaataataatctatcttataaaaatctataaaatctatctttctatttctataattaatttacatatttaactttaaaatttaaaactattttatacaattcaaaaattacttactatatctcccaattcttatctaaaccacgactcttttctctttatacttttataatttaaaaaaatttttaatcataacatctaaattttattttttttccccctccaacctataaatttaatttcaaaatcgaaaaactaaatataaaaaacaaaaaaatcaactccataaaaatatacttcttatacaaaaaacatttaataatctaaacaaaaaaaaaaataataaatattactattacaataacaaaaaaaaaaaaacctcaaaaataacaataattcttaacttaataatcttcaaaaactattaataaaatcctaaaatttaaaaataaattacatttatttaaattttaaatacaatttaaaaaaataataatccattttcaaaaaaattcgttctatcttataccctttaaaaccctaaaaaatactacaaacgttaacctcacattatttttctcttatactttttacaaatattctcataaaaaccgccaaccaatttaaaacttataaaatcgcaccttatatcaacgtaaatactttaaaaataccatttcaacaaacaaatacaacctaatttcaaactatttttaaatataaaaatacctaaatatttacttaaactattaaacatcacctatctctaaacccaaatatcaatatctacataattaaaaaatcattttaaaataatataataaacccactaccctttaaaaacctatacgatattaaaaacctaaatacctaatcccttctatcctcataaaaatccctaaaaaaacctaaaaacaataactaatcctttaaaaatacactaaaaattacccttaaaaaaaaaaaaaaaaaaaaccttttaattaaaattttatcaaatccaatacaaaaattaatcctaaaatatttctaaaacaaccctctaaatacttttctaaatattttctacctcttaatcacattaaataactctaacaaaaaaaaaataaacacaaatactcccaactcatacttttattttttcgttcttttatttttaaatctatttctacaaacaaaacttcatctctaataaaaaaaaaaatcttaatcttcccaaaactaccctacctttacaatcccttaaccataaaaaaacctttaaaaatacattttaaaaaaacaacctacaacaataatttaatcctaaaaaatcccaatactctaaactccccactttccaaacctaaaaacgaaatcctttcccaaccaatatctaaccttccctatcctcaaccaaaacccctacttaacatacctataatatcataaattaactctcaatctccaataacttaaaccgaaaaaacaaataaatataaccataaattaaaaatcgatattaaaaaaatcttaactctcttaataccacaaaactctaacatcctaatacttaactttaaaaacaaaaaaaaataactaaaaaaaaaaaaaaaaataactttaaaaaaaaacctttactactcaaaaaataatctttaaaccaacaacaacaacaccgtctaaaaaattattaaaaattcaaaatctcaaaccccaacctaaaccttacgaatcaaaatctaaattttaacgaaatccctaaataattattataacaataaaaattaaaaataccgactcacaattatacctattataataaaaataaaataacttccctaaataacattaaaaaattacgtaatttacttattaatactccgttattttaataataaaataaaaaatacataacaacatcaaaaaaaaaaaataaaaatcctcacccgcaatctttccaatataccatttcaacccaattcccaaaaaaaacgaccacattcctacaaaaaaaaattcccgaaatccgaaaaaataaacctaccctaaacgaatttcgaaaacccaaaaaacactaaaatatccatataaaaccccaaattcaacaacaaactaaaaaaatcactatcgaaacaaaatacacaaccaaataccctctccccgtacccaaacacaacccaactctctctctaaccaaactaaacgccgttaatactctatttctattctattatcaccctaaaataatcattacaacgtaacgcccttatcctttcaaattcaaacgcaactacaactaaacaacgctataacgcacgcgcaccaccacctacatccgcacctaaccgcgcacgcgccctacataatattcccaacaccgcccttcgaactaccgctcgccacgctaaccgcgaattcgacttccgccgcctcgataataacgaccgcaacaaccgccaaaaccaccaacaaaaactccaacatcgccgatctcaaactaaaaaccaaaaaacacgccgcaaccctaaatctataacgccaacgccaacaccaatatcgcgcctatcccgcgacactcaacctacacgccctccgcgccccgctacttctccgttacccctttaaaacctcgaaaaccgaccctcttcccgcctcactaaccatccctcgtcccctatcgcatcttaaactcgaaaaaccaaactccacgcaaaaccaaaaatctcacgaaacacgcaaactccgtaactcctacccgttttcctactcgaaaacctaaaattaaattttataaaaacgaatttaaaaaaatctaaaaaaaaactaaacaaaaaaatactaaaaccgcgaaatttaactcaccgcaaaactacaacgataaactcttacataaaaaaaaaaatcttattaacaataaaaaaataaacaaacaaaaaaatcgaaaaacaaacgaaaaaaaaaaaaaaaaaaaaaacttatttcttaactactatttaacaaaaactaaaattaaaaaaccaaaaaacaaaaacaaattttaaaattaaaatattttatacatttaaaaatataaaaaaacaacccaaacgattctcgaaaaactaaaaaaaattaccaacttaaatatatatttttaaaaatacgctaaaaaaacaaaacaaaaaaaaaaaatatacttatttaaaaaactaaaataaaaaaaatacgcaactaaaaaattcacaaattttaaaactaacctttttctacgaaattcacgttaacacgaaaaatttaataaaaaaaacgaacctccttttacgttaaatcaaatactttaaatttaaacccaccatatataaaaaaacaaaaaaaaaaaaaatattaaaacgaaaaaaaaaaaaaataatattaacacaaaaaaataccacaaacaataatttctctaaaaaattattataacaaaactatctaaactactaacaataaattccgatttacatattacttatattccattaataatatatctcctcccacccccttatctcccatacactcactccattttcatcttcactataaaaaacaataccaaaaatatctaaaaattaatatatatatatccatatatatatatcatatatttaccatatatatatatatatatatatatatatatatatatatatatttaccctatctttaatcctaaaaaacaaaaaaaaaaaatcaaaaaaaaaaaaaattacactcattatcctaaaaaaacaaaaataaacaaaatatataaaaaaaaaaaaaaaaaaacaaaaccaccaaataaaataataaaaatacaacgcctcgctataccaaacacaataaacgctcaatcaatattaattcccaccattccccttttcttatattccttcttattaatttcctaaaatcctatttaaaaacaataatttatttccccctctctatcccgtcaaattcaccttaaataacacccaactaaatacaaacactaaatttatataaaatatattaatacacacgaacaaaatttattttaactataatatataaactaactttcaacatttacattttatctcacaaaaatatatctattcaaataaccttttttttttatttatttatttcttttttatttttttttttcttttaattatttatttcaattcatataactatttaaactaaaataccttaaactaaaccaatctatatcccaattcgctaacaaacctaaatttataaaattttatttttatttttattttaccttctaatttacaaaaaaaaaaaaaaactcttctaactaaactttaatatacgattaaactttataactatttattctccataaaaacaaaattatttatatttaacatatttttttctaatatattaaattattttaaacaaaaaatattatctcataacgtattatcaatacaaaatatatcgcctccaattctattaaaccttttaaataaataccaaattattaattaaaaacactttacgatcaattaaataaaaatatcgtttcatttaataaaattcgaatcatctttattcctattactattaaaatatccgaaataatctttcc

SEQ ID NO: 4 - DNA, 10550 bp, Artificial Sequence
  SEQ ID NO 1 converted G to A
aaaaaaactatcccgaatattttaataataacaaaaacaaaaataactcgaaccccatcaaataaaacgatattttcattcaattaatcgcaaaatatcttcaattaataacttaacacttatttaaaaaatttaacaaaattaaaaacgacacattttatactaacaacacgtcataaaataacatcttttatttaaaataacttaatacactaaaaaaaaatatatcaaatataaataattttattttcataaaaaacaaataattacaaaactcaaccgcataccaaaattcaattaaaaaaacttttcctctttcttataaattaaaaaataaaacaaaaacaaaaacaaaaccccacaaacttaaacttactaacgaattaaaatacaaactaacttaatccaaaatatcccaatttaaataactacataaattaaaacaaacaaccaaaaaaaaaaaaaaaacaaaaaaaaaacaaacaaacaaaaaaaaaaaattacttaaataaatacacctttataaaataaaatacaaatattaaaaatcaatccacacattataatcaaaataaactttattcgtatatatcaacatatcttacacaaacctaatacctatatctaactaaatattatttaaaataaatttaacgaaataaaaaaaaaaaaataaaccactatcttcaaataaaacttcaaaaaaccaacaaaaaaaaacacaaaaaaaaaaaaataataaaaactaatactaattaaacgcctactatatctaacacaacgaaacgctataccttcattatttcatttaataatcttattttctttttcctttccccacatattctacccacctctatcttcttaaaacaataaatataattttttccctttctaacttttttcttttattccccaaaatcaaaaacaaaacaaatatatatatatatatatatatatatatatatatatatatataacaaatatataatatatatatataaatatatatatatcaatttccaaatacttttaatattatttttcataataaaaataaaaataaaataaatacataaaaaataaaaaaataaaaaaaaacacaccatcaataaaatacaaataacatacaaaccgaaatttactatcaacaatccaaacaattttaccataataatttctcaaaaaaatcattatctataacatttttttatattaatattatttttctctctcctcgttttaatattttctcttttcttactcttccatacataataaatttaaactcaaaacatctaattcaacgtaaaaaaaaacccgcctctctcatcaaatttctcgtattaacgtaaacttcgcaaaaaaaaatcaatttcaaaacctataaacttcccaactacgcacttttttcatcttaattttttaaataaatatacctcttctttctactttaccttcttaacgcatttttaaaaacacacatttaaattaataactccccccaatctctcgaaaatcgtctaaattatttttccatatttttaaatatataaaatactttaattttaaaatttatttttactccttaattctccaatttcaacttctaccaaataacaattaaaaaataaattcccttcctctttttctctcccgtttatctttcgatttttttatttactcattttttcattattaacaaaatttttttttctatacaaaaatccatcgttacaactttacgataaaccaaactccgcgattccaacactcccctatccaatctctctccaaactcccccaaacccgctcctacaaaacccaattctaaaccctcgaataaaaaaacgaacaaaaaccacgaaacctacgtacctcgtaaaatccctaatcctacgtaaaatctaactttccgaatccaaaatacgataaaaaacgaaaaataatcaataaaacgaaaaaaaaaccgactcccgaaatctcaaaaaaataacgaaaaaacaacgaaacgcgaaaaacgtacaaactaaataccgcgaaacaaacgcgacattaatactaacgttaacgtcacaaacccaaaactacgacgtactttttaactttcaatctaaaatcgacgatactaaaattcttactaataatcttaacgactactacgaccgccactaccgaaacgacgaaaaccgaatccgcgaccaacgtaacgaacgacaatccgaaaaacgatactaaaaacatcatataaaacgcgtacgcgaccaaatacgaatacaaataataatacgcgtacgccacaacgctatccaactacaactacgcctaaacctaaaaaaacaaaaacgtcacgttacaataactatcctaaaataacaacaaaataaaaacaaaacattaacgacgtccaacttaaccaaaaaaaaaattaaattatatttaaacacgaaaaaaaaacatttaactatacatcttattccgacaataaccttctcaacctattactaaatctaaaattccacataaatactccaatatcccctaaatctccgaaactcgcttaaaataaacttatttccccgaaccccgaaaacccccttctacaaaaatataaccgtctcccctaaaaactaaactaaaataatatattaaaaaaactacgaataaaaacccttaccctcttctcttaatactaccatacatctcttaccccactaccaaaacaacgaaatatcaacaaacaaactacgcaactttttaatatcattcaaaaaaattactccatcttcaccacaataaacacaattataaatcgatacttctaacctttattaccataacaattacccaaaaatctcgttaaaattcaaattctaattcgcaaaatctaaattaaaacctaaaattctaaatttctaacaatctcccaaacgatattattactactaacccaaaaattaccttttaaataataaaaatctccttttaaaatcacccctccctctttcttcaatcattctctcttatttccaaaaccaaacatcaaaataccaaaatcctataacaccaaaaaaactaaaatctccctaacaccgactctcaattcataatcacacctatctatctccccgatccaaatcactaaaaactaaaaactaattcataacatcacaaacataccaaacaaaaatcctaactaaaaataaaaaaaatcaaacactaattaaaaaaaaatttcgccctcaaacttaaaaaataaaaaattcaaaacactaaaatttcccaaaattaaatcattactacaaactatccttttaaaatatactcttaaaaatttctttataactaaaaaattacaaaaacaaaacaaccctaaaaaaaccaaaacttctctctttaccaaaaataaaactttatttacaaaaacaaatttaaaaacaaaaaaacgaaaaaacaaaaatataaactaaaaatacttatatttatttttcttctatcaaaattatttaatataactaaaaaataaaaaatacctaaaaaaatatctaaaaaattaccttaaaaataccttaaaactaacctctatattaaatttaacaaaatcttaatcaaaaaacttttccttcctctctctctaaaaacaatctttaatatatttttaaaaaaccaattactacccccaaaccccttcaaaaattcccataaaaacaaaaaaaactaaacattcaaacctttaataccgtataaatttttaaaaaataataaatccactatatcatctcaaaataattctccaactatacaaacattaacatctaaactcaaaaacaaataatattcaacaattcaaacaaacattcaaatattttcatatttaaaaacaacttaaaactaaactatacttacctactaaaataacatccttaaaacacctacgttaacataaaatacgactctacaaacttcaaactaactaacgacccctataaaaacacctataaaaaatacaaaaaaaaaataatataaaattaacgtttataacatttttcaaaattccaaaaaatacaaaacaaaacgaattcctttaaaaataaactattatctttctaaactacatccaaaatttaaataaatataacttactcccaaactttaaaactccattaacaacctttaaaaattattaaatcaaaaactattatcatttttaaaattcctttttttctatcattataataataatattcactacttctctctttactcaaactatcaaatattccttatataaaaaacatacctttataaaattaattttcttattttctacatttaactcttcgattttaaaaccaaacctataaattaaaaaaaaaaaaaaaataaaacctaaatattataactaaaaatttttttaaattacaaaaatacaaaaaaaaaaaaatcgtaatttaaacaaaaattaaaaaatacaacaaacaatctttaaattatataaaacaacctcaaattccaaaattaaacatataaattaactacaaaaataaaaaaataaattttataaactttcacaaaataaattactattaatcctaatttcatcttttcacataacaaaatccccataacctaaaactctacttaatacctaataccaacataataataatttaaatatttacccaattaaaacctaaaaaaaatatattcattaataacctataatattaattaacttcatcttaaaaaaaaaccaaacaccaaaacaaataataactcctttacgaaaaaaaaattataaaaattaaaaaaaaataattaatatcataaaaacctcactacaaaaaaatttaaaatttctctattctactatacaacaaaataaaacacaataacaaaataattatacaccattatcataataatctaatttacttctttaaaaaaaaccactatttacaattaaaataataaaaaaacatctaaaaaataactaaaatataaataaaaatataaaacaacaataaaattcacttcttaaaaaaaaaaaaaaaaaacactcaacgaaaaaaaaataaaatatatataaaacaaataactcttaaatactttattctcctaaatattacctacttaaacttaacaacttttacaaacaatataacaaattttttaaaattcaaataaaacttacttttatattccactttcaataataaaacatattaattaactcacaaattaaaatttttaaaaccccattaaaataaaaatactacttcaaaactttcattaatatatttttaaaaacaaatatcataatcaatataaatcttcaaaaatttctctactatttaatacaacaaaaaataaattttataccaaaataacatcctttatataattcacataacctaaaacttcttacctttttttcccctaaaaaaattaaaattaaaatactaataatcataactataataattatcatcatatttttaaaaaattactaaacctctaaaaatctcaatacacaaaacgaaacaaaaaaaacttaaaaaaaaaaactctactactaatactataataaccaaaacaacctattattacttttcctcttcaacgtaactactctatttttttttttcctaatcctattacaaaatataaaaaaaccccttaaaaattatacataattcgctacaaataataatctctatacccaaaaaccgaaaaaatatctattacaacgatactttataacaactacaaatatacgaacctaaaactactccatatattcaaaccaataataactatcattttattctattttattatctaaacgaaaatacaatctaaaacatttaaatttattccccctaaaactaaaaaaatatatatatataaatatacacaatttactcctttaaatacccccaaaacctatttttactattacatttacctccacaaccaataaaaaaaatctatttcaatcctaacttcattaaatataacacgtactaaaaaacaaactttaaaaatataaatactaatatatataatttattttaaaatctaatatatttaaaatcaatttcgttaccttaattaaataaataaacaaaaaataaccaaaatacgtttatttctccgctatttcacacacaatttaaaatcgaccgctaacaacaaactataaatttctacacttaacaattactaattttctttataatctacacatctataaacctaattaaaatatccgcttaacaaacctaaaccctacaaaacaaactttcaaaaaaatccaactccaaaaaacaaaatactctttaaaacttttaaaacatccaaaaataaaacgttttctataaaatttttttttaaaccttataaaaataaacccattaaaattaaaccacattaaaaccaatacgaaacaaaaaaaatataatttataaaaactctaaaaaaaaacccaacacaactttcttcttctaaaaacatatattaaaataaaaaaaaaaaaaaaaaaaaaaatactataaaactaaaatctactttaaaaaataaaacctctaataaaaatatctctttccttctcatcttacaccaaacactaaaaacaccacctcccgaatatatcccccaaacctctttccaaccccaaactctctctactccctaaaccctcgacctcctaacaactaaatacctacactcgaacctccgacaaacccaatcgctaactcaattcctctcgcataaaaacgtcgaaataataaatctcgtcaaaaaacctctccaactcattaaattattccaaaataaaattaatccgacttcgcctctacttaattttaatctaaccttcgtcctccatccctttcgcatcctctttacgatctttcaactccgaaaacactaaaaaaaacaccccaacgaaaccacacgtacatccacacgaacacacacacacacgcacacacacacgaacgaaaacaacacaacaaatcctattaccaaatttatcgcaaaaaaaaataaaaaatccttccccctcgtaaaatcctaatccgacttctcaacccacccgaaaaaaaaatatcccgaaaaaacgcaaaaatcaaatctaaacgaccgcctaaaacaaaataaacaatcatccaccacctccttcacaaacgtacctcgactcaccgcctcactattcaccccccaatcaccgaccttccccaacaaacaccaaataatttccactcactttcgcactcacttactccgcaacactcacaaaaatcaaaaacgcaacgccaaacctctcacaaaacaaaatctaaacataaaaacgcgacaaaaccacccactaacctctcacaccgaccattcccaaaaaactaactcgttctcaaaccaactctcaccaccgctaactctctacctaccgcaaacttactaatctaatttaaaaacaattaaaccgaaaaatatcaacgaaaacaacaaaccccgatattataccgcacaaaaaaccgcatccgcaaacgcccctcgctacccctaaactcgaaccaaaccctacataaaatcccctaaacaaccaaataatctccgtcccgcctacccgaccgaaatcgcacgaacacaaacgcccacgccatattaactacccaaaaaactcgccgcccaaaccgaaccaaaaaacaaaaaacgaaaaaccaacctccgataacgaacgaaaacaaccgctctttctattctctcttcgccctccctcgtaaaaacgcaaactcgaccctaaacgcttaacccacaaaaatcaacaaattcaaacgaaatattcgcgatcctcgatttctattaattactcaaaaccttttcatacaaccaacaactcgaatatttaataaaatataaatttttctactctaaatttacacacatccgttaccactttcctatccttctcctccctttaaaaacaaaaactcttctaatataaacatatacaaccaccatatatatatccccctaaaacctttatcctctacgcccgaatcaaaaactccgaattttaaaaaatcaaaactaaatttaaataatcgaaactatttacaattcttcccaacgcaaaatccaaaccaacataaatattaaaaatcatttccgaaaaaaccgatctacaaaaaaattaatacttaccctaaacttaaaaatcaacaatcccaaaaactacgcccgccgaaaattcaaacctaaacctcgccgtcaaacgcgctaacgaacctataattcgaacctataattataaactcataaccgaaaaactacactaaacaccttaacgataactccaccactttacaaatttaaaaaaacctaatactaatttaaaaaaaaaaaaatcgctaaatctcgaccctaccgaacattctaactacgataatcttaaatattaccgtattttctaattctcaaaattaattttataaacatttatcctttctttaaaaaaaaacattataataaacattataaaccaatttaataacaaaatatttttactaatattatattaaaatttaaatataaaaacacacccttcaaattctaccaataaaataaaaaaactttatctattacatatcatctacacaataaataccactcaaaattttaaaacttcgaaaaaatatccaaatttcaaattaatccataaacttaataaaatctctatttttatccccaaatttccaattactaatctaaacaaaaacaaaccgtatcataaacaacttaattacttacttaaatatataattccacgcataccacatattttcttaattaaaaaaatataacaatacaccaattactacaaacaccaaataccaaatcaattttattattaaactcctttaaattaatataaaacgctctaccattaaaataaaaacgtttcctaacactaaactaacaccaaaaaatttttactctaaaaaaaccgaatactaaataattaaaaacactaaaaaaattatataaatataaatccccacctcccccgccgccccccccccccgcccccaacacaccaaaaaaccgaaacgcctcgcccgaaaaaaacaacgtaacctaaacctcaactttcgttaacttccttctaccttaaaaaaaaataaaaaattttaacccctaaaatccaaaaattcaaaaactctacgaaattcgaaaaatcttaacgaccccaaacacctaaacgaccgaaaaattaaactttccacctctaccccttctccctcccgacaaactctacgctaaaaactaactttaatcccgccgtctaattcaacaccccctcctacaacccgaccccgcgaacttccacgtccgcctacgaaccatccgaactacgaacccatcctcccgctaataaaaaatacatttcgataaaataaaaacatctaaaaaaaacgcgcacaaaccccacacgcttccccccacgccacccccacaacacactaaaaccccacccataaccgaacataccaccgaacccccacccttccactatcactctaactaacaaaactaaaatccaaaaccctctccgtatccctctcccctccccgctaaacctcgaaatcctctcccgcccgaaaaaaatccgaaaaacgaaaaaaaaaaccgctaccgaaaatcaatcaaatcgttaccctccgtcaatcgcgaactacccgactccctacttctctcgacgacgcccatatccaactcccgaacgaaaaaacgccctcctccaactcctccgcctactcctcctcctcctacacctcctccgcctcctccgccgccgcctccgcctcctccgccgccgcctccgccgaccgcccgaactaccgaactactacgatcgtcgcgacccgcctcgatacaaccgatcgactccttaaccccgcgcaacgacccgctctccaacacctcccgatacgtaatcgcctccttcttctccttcactttctaatcaaaaaacttaaaaacgaacgccgtaaattcttccatcgccgccgcacgtcaacccgacgctcaacctctaccaacaaaaccccgctctttttttccttcttctttttttactactccaacccccccaataataacacatcaataaaacaaaaaataaaaaaaaaaaaaaaaaaaaaaaaaaaaaaaaaaaaaaaaaaaaaaaaaaaaaaaaaaaaaaaaaaaataaaaaaaaaaaaaaaaataaaaaaaaaaaaaaaaaaaaaaaaaaaaaaaaaaaaaaaaaaataaaaaaaaaaaaaaaaaaaaaaaaaaaaaaaaaaaaaacgaaaactaccgaaaaaaaataaaaactaatacttccctaccgaaacgcgcttattcaatattaaaatctttaacaattcttcctacgaaaaattcaaatctaataacgacgctacgcttaaaatcccactatttatacttcctaacgacgacctcttattttaaataaattacctcccctcgcaactcga

SEQ ID NO: 5 - DNA, 10550 bp, Artificial Sequence
  SEQ ID NO 1 converted G to A, reverse complement
tcgagttgcgaggggaggtaatttatttaaaataagaggtcgtcgttaggaagtataaatagtgggattttaagcgtagcgtcgttattagatttgaatttttcgtaggaagaattgttaaagattttaatattgaataagcgcgtttcggtagggaagtattagtttttattttttttcggtagttttcgttttttttttttttttttttttttttttttttttttttatttttttttttttttttttttttttttttttttttttttttttatttttttttttttttttattttttttttttttttttttttttttttttttttttttttttttttttttttttttttttttttttttattttttgttttattgatgtgttattattgggggggttggagtagtaaaaaaagaagaaggaaaaaaagagcggggttttgttggtagaggttgagcgtcgggttgacgtgcggcggcgatggaagaatttacggcgttcgtttttaagttttttgattagaaagtgaaggagaagaaggaggcgattacgtatcgggaggtgttggagagcgggtcgttgcgcggggttaaggagtcgatcggttgtatcgaggcgggtcgcgacgatcgtagtagttcggtagttcgggcggtcggcggaggcggcggcggaggaggcggaggcggcggcggaggaggcggaggaggtgtaggaggaggaggagtaggcggaggagttggaggagggcgttttttcgttcgggagttggatatgggcgtcgtcgagagaagtagggagtcgggtagttcgcgattgacggagggtaacgatttgattgattttcggtagcggttttttttttcgtttttcggatttttttcgggcgggagaggatttcgaggtttagcggggaggggagagggatacggagagggttttggattttagttttgttagttagagtgatagtggaagggtgggggttcggtggtatgttcggttatgggtggggttttagtgtgttgtgggggtggcgtggggggaagcgtgtggggtttgtgcgcgttttttttagatgtttttattttatcgaaatgtattttttattagcgggaggatgggttcgtagttcggatggttcgtaggcggacgtggaagttcgcggggtcgggttgtaggagggggtgttgaattagacggcgggattaaagttagtttttagcgtagagtttgtcgggagggagaaggggtagaggtggaaagtttaatttttcggtcgtttaggtgtttggggtcgttaagatttttcgaatttcgtagagtttttgaatttttggattttaggggttaaaattttttatttttttttaaggtagaaggaagttaacgaaagttgaggtttaggttacgttgtttttttcgggcgaggcgtttcggttttttggtgtgttgggggcggggggggggggcggcgggggaggtggggatttatatttatataatttttttagtgtttttaattatttagtattcggtttttttagagtaaaaattttttggtgttagtttagtgttaggaaacgtttttattttaatggtagagcgttttatattaatttaaaggagtttaataataaaattgatttggtatttggtgtttgtagtaattggtgtattgttatatttttttaattaagaaaatatgtggtatgcgtggaattatatatttaagtaagtaattaagttgtttatgatacggtttgtttttgtttagattagtaattggaaatttggggataaaaatagagattttattaagtttatggattaatttgaaatttggatattttttcgaagttttaaaattttgagtggtatttattgtgtagatgatatgtaatagataaagtttttttattttattggtagaatttgaagggtgtgtttttatatttaaattttaatataatattagtaaaaatattttgttattaaattggtttataatgtttattataatgtttttttttaaagaaaggataaatgtttataaaattaattttgagaattagaaaatacggtaatatttaagattatcgtagttagaatgttcggtagggtcgagatttagcgatttttttttttttaaattagtattaggtttttttaaatttgtaaagtggtggagttatcgttaaggtgtttagtgtagtttttcggttatgagtttataattataggttcgaattataggttcgttagcgcgtttgacggcgaggtttaggtttgaattttcggcgggcgtagtttttgggattgttgatttttaagtttagggtaagtattaatttttttgtagatcggttttttcggaaatgatttttaatatttatgttggtttggattttgcgttgggaagaattgtaaatagtttcgattatttaaatttagttttgattttttaaaattcggagtttttgattcgggcgtagaggataaaggttttagggggatatatatatggtggttgtatatgtttatattagaagagtttttgtttttaaagggaggagaaggataggaaagtggtaacggatgtgtgtaaatttagagtagaaaaatttatattttattaaatattcgagttgttggttgtatgaaaaggttttgagtaattaatagaaatcgaggatcgcgaatatttcgtttgaatttgttgatttttgtgggttaagcgtttagggtcgagtttgcgtttttacgagggagggcgaagagagaatagaaagagcggttgttttcgttcgttatcggaggttggtttttcgttttttgttttttggttcggtttgggcggcgagttttttgggtagttaatatggcgtgggcgtttgtgttcgtgcgatttcggtcgggtaggcgggacggagattatttggttgtttaggggattttatgtagggtttggttcgagtttaggggtagcgaggggcgtttgcggatgcggttttttgtgcggtataatatcggggtttgttgttttcgttgatatttttcggtttaattgtttttaaattagattagtaagtttgcggtaggtagagagttagcggtggtgagagttggtttgagaacgagttagttttttgggaatggtcggtgtgagaggttagtgggtggttttgtcgcgtttttatgtttagattttgttttgtgagaggtttggcgttgcgtttttgatttttgtgagtgttgcggagtaagtgagtgcgaaagtgagtggaaattatttggtgtttgttggggaaggtcggtgattggggggtgaatagtgaggcggtgagtcgaggtacgtttgtgaaggaggtggtggatgattgtttattttgttttaggcggtcgtttagatttgatttttgcgttttttcgggatatttttttttcgggtgggttgagaagtcggattaggattttacgagggggaaggattttttatttttttttgcgataaatttggtaataggatttgttgtgttgttttcgttcgtgtgtgtgtgcgtgtgtgtgtgtgttcgtgtggatgtacgtgtggtttcgttggggtgttttttttagtgttttcggagttgaaagatcgtaaagaggatgcgaaagggatggaggacgaaggttagattaaaattaagtagaggcgaagtcggattaattttattttggaataatttaatgagttggagaggttttttgacgagatttattatttcgacgtttttatgcgagaggaattgagttagcgattgggtttgtcggaggttcgagtgtaggtatttagttgttaggaggtcgagggtttagggagtagagagagtttggggttggaaagaggtttgggggatatattcgggaggtggtgtttttagtgtttggtgtaagatgagaaggaaagagatatttttattagaggttttattttttaaagtagattttagttttatagtatttttttttttttttttttttttattttaatatatgtttttagaagaagaaagttgtgttgggtttttttttagagtttttataaattatattttttttgtttcgtattggttttaatgtggtttaattttaatgggtttatttttataaggtttaaaaaaaaattttatagaaaacgttttatttttggatgttttaaaagttttaaagagtattttgttttttggagttggatttttttgaaagtttgttttgtagggtttaggtttgttaagcggatattttaattaggtttatagatgtgtagattataaagaaaattagtaattgttaagtgtagaaatttatagtttgttgttagcggtcgattttaaattgtgtgtgaaatagcggagaaataaacgtattttggttattttttgtttatttatttaattaaggtaacgaaattgattttaaatatattagattttaaaataaattatatatattagtatttatatttttaaagtttgttttttagtacgtgttatatttaatgaagttaggattgaaatagattttttttattggttgtggaggtaaatgtaatagtaaaaataggttttgggggtatttaaaggagtaaattgtgtatatttatatatatatatttttttagttttagggggaataaatttaaatgttttagattgtattttcgtttagataataaaatagaataaaatgatagttattattggtttgaatatatggagtagttttaggttcgtatatttgtagttgttataaagtatcgttgtaatagatattttttcggtttttgggtatagagattattatttgtagcgaattatgtataatttttaaggggtttttttatattttgtaataggattaggaaaaaaaaaaatagagtagttacgttgaagaggaaaagtaataataggttgttttggttattatagtattagtagtagagtttttttttttaagttttttttgtttcgttttgtgtattgagatttttagaggtttagtaattttttaaaaatatgatgataattattatagttatgattattagtattttaattttaatttttttaggggaaaaaaaggtaagaagttttaggttatgtgaattatataaaggatgttattttggtataaaatttattttttgttgtattaaatagtagagaaatttttgaagatttatattgattatgatatttgtttttaaaaatatattaatgaaagttttgaagtagtatttttattttaatggggttttaaaaattttaatttgtgagttaattaatatgttttattattgaaagtggaatataaaagtaagttttatttgaattttaaaaaatttgttatattgtttgtaaaagttgttaagtttaagtaggtaatatttaggagaataaagtatttaagagttatttgttttatatatattttatttttttttcgttgagtgttttttttttttttttttaagaagtgaattttattgttgttttatatttttatttatattttagttattttttagatgtttttttattattttaattgtaaatagtggttttttttaaagaagtaaattagattattatgataatggtgtataattattttgttattgtgttttattttgttgtatagtagaatagagaaattttaaatttttttgtagtgaggtttttatgatattaattatttttttttaatttttataatttttttttcgtaaaggagttattatttgttttggtgtttggtttttttttaagatgaagttaattaatattataggttattaatgaatatattttttttaggttttaattgggtaaatatttaaattattattatgttggtattaggtattaagtagagttttaggttatggggattttgttatgtgaaaagatgaaattaggattaatagtaatttattttgtgaaagtttataaaatttatttttttatttttgtagttaatttatatgtttaattttggaatttgaggttgttttatataatttaaagattgtttgttgtattttttaatttttgtttaaattacgatttttttttttttgtatttttgtaatttaaaaaaatttttagttataatatttaggttttattttttttttttttttaatttataggtttggttttaaaatcgaagagttaaatgtagaaaataagaaaattaattttataaaggtatgttttttatataaggaatatttgatagtttgagtaaagagagaagtagtgaatattattattataatgatagaaaaaaaggaattttaaaaatgataatagtttttgatttaataatttttaaaggttgttaatggagttttaaagtttgggagtaagttatatttatttaaattttggatgtagtttagaaagataatagtttatttttaaaggaattcgttttgttttgtattttttggaattttgaaaaatgttataaacgttaattttatattattttttttttgtattttttataggtgtttttataggggtcgttagttagtttgaagtttgtagagtcgtattttatgttaacgtaggtgttttaaggatgttattttagtaggtaagtatagtttagttttaagttgtttttaaatatgaaaatatttgaatgtttgtttgaattgttgaatattatttgtttttgagtttagatgttaatgtttgtatagttggagaattattttgagatgatatagtggatttattattttttaaaaatttatacggtattaaaggtttgaatgtttagttttttttgtttttatgggaatttttgaaggggtttgggggtagtaattggttttttaaaaatatattaaagattgtttttagagagagaggaaggaaaagttttttgattaagattttgttaaatttaatatagaggttagttttaaggtatttttaaggtaattttttagatatttttttaggtattttttattttttagttatattaaataattttgatagaagaaaaataaatataagtatttttagtttatatttttgttttttcgtttttttgtttttaaatttgtttttgtaaataaagttttatttttggtaaagagagaagttttggtttttttagggttgttttgtttttgtaattttttagttataaagaaatttttaagagtatattttaaaaggatagtttgtagtaatgatttaattttgggaaattttagtgttttgaattttttattttttaagtttgagggcgaaatttttttttaattagtgtttgattttttttatttttagttaggatttttgtttggtatgtttgtgatgttatgaattagtttttagtttttagtgatttggatcggggagatagataggtgtgattatgaattgagagtcggtgttagggagattttagtttttttggtgttataggattttggtattttgatgtttggttttggaaataagagagaatgattgaagaaagagggaggggtgattttaaaaggagatttttattatttaaaaggtaatttttgggttagtagtaataatatcgtttgggagattgttagaaatttagaattttaggttttaatttagattttgcgaattagaatttgaattttaacgagatttttgggtaattgttatggtaataaaggttagaagtatcgatttataattgtgtttattgtggtgaagatggagtaatttttttgaatgatattaaaaagttgcgtagtttgtttgttgatatttcgttgttttggtagtggggtaagagatgtatggtagtattaagagaagagggtaagggtttttattcgtagtttttttaatatattattttagtttagtttttaggggagacggttatatttttgtagaagggggttttcggggttcggggaaataagtttattttaagcgagtttcggagatttaggggatattggagtatttatgtggaattttagatttagtaataggttgagaaggttattgtcggaataagatgtatagttaaatgtttttttttcgtgtttaaatataatttaattttttttttggttaagttggacgtcgttaatgttttgtttttattttgttgttattttaggatagttattgtaacgtgacgtttttgtttttttaggtttaggcgtagttgtagttggatagcgttgtggcgtacgcgtattattatttgtattcgtatttggtcgcgtacgcgttttatatgatgtttttagtatcgtttttcggattgtcgttcgttacgttggtcgcggattcggttttcgtcgtttcggtagtggcggtcgtagtagtcgttaagattattagtaagaattttagtatcgtcgattttagattgaaagttaaaaagtacgtcgtagttttgggtttgtgacgttaacgttagtattaatgtcgcgtttgtttcgcggtatttagtttgtacgtttttcgcgtttcgttgttttttcgttatttttttgagatttcgggagtcggtttttttttcgttttattgattatttttcgttttttatcgtattttggattcggaaagttagattttacgtaggattagggattttacgaggtacgtaggtttcgtggtttttgttcgtttttttattcgagggtttagaattgggttttgtaggagcgggtttgggggagtttggagagagattggataggggagtgttggaatcgcggagtttggtttatcgtaaagttgtaacgatggatttttgtatagaaaaaaaaattttgttaataatgaaaaaatgagtaaataaaaaaatcgaaagataaacgggagagaaaaagaggaagggaatttattttttaattgttatttggtagaagttgaaattggagaattaaggagtaaaaataaattttaaaattaaagtattttatatatttaaaaatatggaaaaataatttagacgattttcgagagattggggggagttattaatttaaatgtgtgtttttaaaaatgcgttaagaaggtaaagtagaaagaagaggtatatttatttaaaaaattaagatgaaaaaagtgcgtagttgggaagtttataggttttgaaattgatttttttttgcgaagtttacgttaatacgagaaatttgatgagagaggcgggtttttttttacgttgaattagatgttttgagtttaaatttattatgtatggaagagtaagaaaagagaaaatattaaaacgaggagagagaaaaataatattaatataaaaaaatgttatagataatgatttttttgagaaattattatggtaaaattgtttggattgttgatagtaaatttcggtttgtatgttatttgtattttattgatggtgtgtttttttttatttttttattttttatgtatttattttatttttatttttattatgaaaaataatattaaaagtatttggaaattgatatatatatatttatatatatatattatatatttgttatatatatatatatatatatatatatatatatatatatatatttgttttgtttttgattttggggaataaaagaaaaaagttagaaagggaaaaaattatatttattgttttaagaagatagaggtgggtagaatatgtggggaaaggaaaaagaaaataagattattaaatgaaataatgaaggtatagcgtttcgttgtgttagatatagtaggcgtttaattagtattagtttttattatttttttttttttgtgtttttttttgttggttttttgaagttttatttgaagatagtggtttatttttttttttttatttcgttaaatttattttaaataatatttagttagatataggtattaggtttgtgtaagatatgttgatatatacgaataaagtttattttgattataatgtgtggattgatttttaatatttgtattttattttataaaggtgtatttatttaagtaattttttttttttgtttgtttgttttttttttgttttttttttttttttggttgtttgttttaatttatgtagttatttaaattgggatattttggattaagttagtttgtattttaattcgttagtaagtttaagtttgtggggttttgtttttgtttttgttttattttttaatttataagaaagaggaaaagtttttttaattgaattttggtatgcggttgagttttgtaattatttgttttttatgaaaataaaattatttatatttgatatatttttttttagtgtattaagttattttaaataaaagatgttattttatgacgtgttgttagtataaaatgtgtcgtttttaattttgttaaattttttaaataagtgttaagttattaattgaagatattttgcgattaattgaatgaaaatatcgttttatttgatggggttcgagttatttttgtttttgttattattaaaatattcgggatagttttttt

SEQ ID NO: 6 - DNA, 5473 bp, Homo sapiens
gtaatgttaattagcttcatcttaaagaaaaaccaaacaccagagcaaataatgactcctttgcggagaaggagttgtaagggttggggagaggtaattagtgtcataaaagcctcactacaaagaagtttagaatttctctgttctgctgtgcagcagagtgaaacacagtagcagaataattgtgcaccattatcatgataatctaatttgcttctttagaaaaagccactgtttgcagttaggatagtaggaaaacatctagaaaataactaaaatgtaaataaaagtatgaaacaacaataaagttcacttcttaagagaaaaaaagaagagcactcagcgaaaggagaatgaagtatatataaagcaagtaactcttaggtactttgttctcctggatgttacctgcttaaacttgacaacttttacaggcagtatgacagattttttaaaattcaaataaagcttgcttttgtattccactttcaataataaagcatattgattgactcacaaattaaagtttttaaaaccccattaaagtaaaaatactgcttcagagctttcattggtatgtttttgaaagcaagtgtcatagtcaatgtaaatcttcaaaaatttctctgctatttagtacaacagagggtggattttgtgccagggtggcatcctttgtgtaattcacatggcctgaggcttcttgcctttttttcccctagaagagttggaattaagatactaataatcataactgtaataattatcatcatgtttttaaggggttgctgagcctctggagatctcagtgcacagagcgaagcaggagaggcttaaaaaggaaggctctgctgctgatgctgtgatgaccaaggcagcctattattacttttcctcttcaacgtgactgctctgtttttttttttcctgatcctgttacaagatgtggggaagccccttaagaattgtgcatagttcgctacagatggtggtctctgtgcccagagaccgaaagagtatctgttacagcgatactttgtaacagctgcagatgtgcgggcctaaaactgctccatgtgttcaaaccaataataactatcattttattctattttattatctgagcggaaatacaatctgaaacatttaaatttgttccccctaaagctagaaggatatgtgtatgtgaatgtacacaatttgctcctttaaatgcccccaaagcctatttttgctattacatttgcctccacaaccaataaagaaaatctgtttcagtcctaacttcattgggtatggcacgtgctggagggcaggctttggaaatataaatactaatatatgtggtttgttttaaaatctaatgtatttagggtcagtttcgttgccttggttaaatgaatgaacaaagagtgaccaaaatgcgtttatttctccgctatttcacacacagtttgagatcgaccgctaacagcagactgtaaatttctgcacttaacagttactaattttctttatagtctgcacatctataaacctaattgggatatccgcttgacaaacctagaccctgcagagcagactttcaaggaaatccaactccagagaacaaaatactctttggaacttttaaggcatccagagataaaacgttttctgtgggatttttttttaagccttataaaagtaggcccattaggattaaaccacattaaagccagtgcgaggcaaggggggtgtggtttatagaaactctgggagaaagcccagcacagctttcttcttctgggaacatatgttaggatggaaaaaaaaaaaaggaggggagtgctataggactaagatctactttggaaaataagacctctagtaaagatatctctttccttctcatcttacaccagacactagaagcaccacctcccgagtgtgtcccccaagcctctttccaaccccaggctctctctgctccctgggccctcgacctcctggcagctgggtacctgcactcgggcctccgacaggcccagtcgctggctcagttcctctcgcatgaaggcgtcgggatagtgggtctcgtcaaaaagcctctccagctcattgagttgttccagggtgaaattggtccgacttcgcctctgcttgattttggtctggccttcgtcctccatccctttcgcatcctctttgcgatctttcagctccggggacactggagggggcaccccagcggggccacacgtgcatccacacgaacacacacacacacgcacacacacacggacgaaaacagcacagcaaatcctgttaccagatttgtcgcaaaaaagaatagagaatccttccccctcgtggaatcctggtccggcttctcaacccacccggaggaagaatatcccggagaagcgcaaaggtcaagtctgagcggccgcctggggcaaggtgggcagtcatccaccacctccttcacaaacgtacctcgactcaccgcctcactattcaccccccagtcaccggccttccccagcaaacaccaaatggtttccactcactttcgcactcacttgctccgcagcactcacagaaatcaaaggcgcagcgccaggcctctcacagagcaaagtctggacataagggcgcggcaaggccacccactggcctctcacaccggccattcccagaaggctggctcgttctcaggccagctctcaccaccgctggctctctgcctaccgcaaacttgctggtctaatttaggaacaattgggccgaaaggtatcagcgagagcaacagaccccggtgttgtgccgcacagggagccgcatccgcagacgcccctcgctgcccctgggctcgggccaaaccctgcataaggtcccctggacagccaggtaatctccgtcccgcctgcccgaccggggtcgcacgagcacaggcgcccacgccatgttggctgcccaaagggctcgccgcccaagccgggccagaaggcaggaggcggaaaaccagcctccggtggcgggcgaaagcaaccgctctttctgttctctcttcgccctccctcgtggaaacgcagactcgaccctaaacgcttaacccacagagatcaacaggttcaagcggaatattcgcgatcctcggtttctattggttgctcaaagccttttcatgcaaccagcagctcggatgtttaataaaatatgaatttttctgctctgggtttgcacacatccgttgccactttcctgtccttctcctccctttaaaaacaggagctcttctagtgtgagcatatacagccaccatatatatatccccctggaacctttatcctctgcgcccgaatcagagactccgggttttaagaagtcaaagctgagtttagatagtcgaggctgtttgcagttcttcccagcgcaggatccaggccagcatgggtgttaaaaatcatttccggaagagccggtctgcagagaaattaatacttgccctgagcttggaaatcaacaatcccaggaactgcgcccgccgggggttcagacctgaacctcgccgtcaggcgcgctggcgaacctgtagttcgggcctgtagttgtggactcatggccgggaggctgcactgggcaccttgacggtaactccaccactttacaaatttaggaagacctggtgctggtttggaggaagaagagtcgctagatctcggccctgccgggcattctagctacggtgatcttaaatattgccgtattttctagttctcagagttagttttgtggacatttgtcctttctttaggaaggaacattatagtaaacattatgggccaatttaatagcaaagtatttttactgatattgtattgaaatttaaatatgaaaacacacccttcaaattctgccaatgaggtgggaaggctttgtctattacatgtcatctacacagtggatgccactcaaagttttggggcttcgaggagatgtccaaatttcaagttgatccatagacttaataaaatctctatttttgtccccaaatttccaattactgatctaaacaggaacagaccgtgtcataaacaacttaattgcttgcttaaatatgtgattccacgcatgccacatattttcttaattggaaaaatatggcagtacaccagttactgcaggcaccaagtgccaaatcaattttattgttgaactcctttgaattggtatagaacgctctgccattgagataaagacgtttcctaacactgagctagcaccaaaggatttttgctctaagagaaccgagtactgggtgattgaaaacactaaaggaattgtgtaaatatagatccccacctcccccgccgccccccccccccgcccccaacacaccaagaaaccgaaacgcctcgcccgggagaagcagcgtagcctggacctcagctttcgttggcttccttctaccttgaaagagaatgggaagttttggcccctaagatccaaagattcaggggctctgcggagttcgaggggtcttggcggccccaaacacctaggcgaccggagggttaagctttccacctctgccccttctccctcccggcaaactctgcgctagaggctagctttagtcccgccgtctggttcagcaccccctcctgcagcccggccccgcgaacttccacgtccgcctgcgggccatccgggctgcgggcccatcctcccgctggtgaaaaatgcatttcggtggaatgggaacatctgggaagggcgcgcacaaaccccacacgcttccccccacgccacccccacaacacactaggaccccacccatgaccgagcataccaccggacccccacccttccactatcactctagctgacaaagctggggtccagggccctctccgtgtccctctcccctccccgctgggcctcggagtcctctcccgcccgagaggagtccggaaggcgggaaggggaaccgctgccgggggtcagtcaggtcgttaccctccgtcagtcgcgggctgcccggctccctgcttctctcggcggcgcccatgtccagctcccggacgggagagcgccctcctccagctcctccgcctgctcctcctcctcctacacctcctccgcctcctccgccgccgcctccgcctcctccgccgccgcctccgccggccgcccggactgccgggctgctgcggtcgtcgcggcccgcctcggtgcagccggtcggctccttggccccgcgcagcggcccgctctccagcacctcccggtacgtgatcgcctccttcttctccttcactttctggtcaaaagacttggagacgaacgccgtaagttcttccatcgccgccgcacgtcagcccggcgctcaacctctgccagcagagccccgctctttttttccttcttctttttttactgctccagcccccccaataataacacatcaatgggacaggaggtgggg

SEQ ID NO: 7 - DNA, 5473 bp, Artificial Sequence
  SEQ ID NO 6 converted C to T
gtaatgttaattagttttattttaaagaaaaattaaatattagagtaaataatgatttttttgcggagaaggagttgtaagggttggggagaggtaattagtgttataaaagttttattataaagaagtttagaatttttttgttttgttgtgtagtagagtgaaatatagtagtagaataattgtgtattattattatgataatttaatttgtttttttagaaaaagttattgtttgtagttaggatagtaggaaaatatttagaaaataattaaaatgtaaataaaagtatgaaataataataaagtttattttttaagagaaaaaaagaagagtatttagcgaaaggagaatgaagtatatataaagtaagtaatttttaggtattttgtttttttggatgttatttgtttaaatttgataatttttataggtagtatgatagattttttaaaatttaaataaagtttgtttttgtattttatttttaataataaagtatattgattgatttataaattaaagtttttaaaattttattaaagtaaaaatattgttttagagtttttattggtatgtttttgaaagtaagtgttatagttaatgtaaatttttaaaaatttttttgttatttagtataatagagggtggattttgtgttagggtggtattttttgtgtaatttatatggtttgaggttttttgttttttttttttttagaagagttggaattaagatattaataattataattgtaataattattattatgtttttaaggggttgttgagtttttggagattttagtgtatagagcgaagtaggagaggtttaaaaaggaaggttttgttgttgatgttgtgatgattaaggtagtttattattattttttttttttaacgtgattgttttgttttttttttttttgattttgttataagatgtggggaagttttttaagaattgtgtatagttcgttatagatggtggtttttgtgtttagagatcgaaagagtatttgttatagcgatattttgtaatagttgtagatgtgcgggtttaaaattgttttatgtgtttaaattaataataattattattttattttattttattatttgagcggaaatataatttgaaatatttaaatttgttttttttaaagttagaaggatatgtgtatgtgaatgtatataatttgtttttttaaatgtttttaaagtttatttttgttattatatttgtttttataattaataaagaaaatttgttttagttttaattttattgggtatggtacgtgttggagggtaggttttggaaatataaatattaatatatgtggtttgttttaaaatttaatgtatttagggttagtttcgttgttttggttaaatgaatgaataaagagtgattaaaatgcgtttattttttcgttattttatatatagtttgagatcgatcgttaatagtagattgtaaatttttgtatttaatagttattaattttttttatagtttgtatatttataaatttaattgggatattcgtttgataaatttagattttgtagagtagatttttaaggaaatttaattttagagaataaaatattttttggaatttttaaggtatttagagataaaacgttttttgtgggatttttttttaagttttataaaagtaggtttattaggattaaattatattaaagttagtgcgaggtaaggggggtgtggtttatagaaattttgggagaaagtttagtatagttttttttttttgggaatatatgttaggatggaaaaaaaaaaaaggaggggagtgttataggattaagatttattttggaaaataagatttttagtaaagatattttttttttttttattttatattagatattagaagtattatttttcgagtgtgttttttaagtttttttttaattttaggttttttttgttttttgggttttcgattttttggtagttgggtatttgtattcgggttttcgataggtttagtcgttggtttagtttttttcgtatgaaggcgtcgggatagtgggtttcgttaaaaagtttttttagtttattgagttgttttagggtgaaattggttcgatttcgtttttgtttgattttggtttggttttcgttttttatttttttcgtattttttttgcgattttttagtttcggggatattggagggggtattttagcggggttatacgtgtatttatacgaatatatatatatacgtatatatatacggacgaaaatagtatagtaaattttgttattagatttgtcgtaaaaaagaatagagaatttttttttttcgtggaattttggttcggttttttaatttattcggaggaagaatatttcggagaagcgtaaaggttaagtttgagcggtcgtttggggtaaggtgggtagttatttattattttttttataaacgtatttcgatttatcgttttattatttattttttagttatcggttttttttagtaaatattaaatggtttttatttattttcgtatttatttgtttcgtagtatttatagaaattaaaggcgtagcgttaggttttttatagagtaaagtttggatataagggcgcggtaaggttatttattggttttttatatcggttatttttagaaggttggttcgtttttaggttagtttttattatcgttggttttttgtttatcgtaaatttgttggtttaatttaggaataattgggtcgaaaggtattagcgagagtaatagatttcggtgttgtgtcgtatagggagtcgtattcgtagacgtttttcgttgtttttgggttcgggttaaattttgtataaggttttttggatagttaggtaattttcgtttcgtttgttcgatcggggtcgtacgagtataggcgtttacgttatgttggttgtttaaagggttcgtcgtttaagtcgggttagaaggtaggaggcggaaaattagttttcggtggcgggcgaaagtaatcgttttttttgtttttttttcgttttttttcgtggaaacgtagattcgattttaaacgtttaatttatagagattaataggtttaagcggaatattcgcgattttcggtttttattggttgtttaaagtttttttatgtaattagtagttcggatgtttaataaaatatgaatttttttgttttgggtttgtatatattcgttgttatttttttgttttttttttttttttaaaaataggagtttttttagtgtgagtatatatagttattatatatatatttttttggaatttttattttttgcgttcgaattagagatttcgggttttaagaagttaaagttgagtttagatagtcgaggttgtttgtagttttttttagcgtaggatttaggttagtatgggtgttaaaaattattttcggaagagtcggtttgtagagaaattaatatttgttttgagtttggaaattaataattttaggaattgcgttcgtcgggggtttagatttgaatttcgtcgttaggcgcgttggcgaatttgtagttcgggtttgtagttgtggatttatggtcgggaggttgtattgggtattttgacggtaattttattattttataaatttaggaagatttggtgttggtttggaggaagaagagtcgttagatttcggttttgtcgggtattttagttacggtgattttaaatattgtcgtattttttagtttttagagttagttttgtggatatttgttttttttttaggaaggaatattatagtaaatattatgggttaatttaatagtaaagtatttttattgatattgtattgaaatttaaatatgaaaatatattttttaaattttgttaatgaggtgggaaggttttgtttattatatgttatttatatagtggatgttatttaaagttttggggtttcgaggagatgtttaaattttaagttgatttatagatttaataaaatttttatttttgtttttaaatttttaattattgatttaaataggaatagatcgtgttataaataatttaattgtttgtttaaatatgtgattttacgtatgttatatatttttttaattggaaaaatatggtagtatattagttattgtaggtattaagtgttaaattaattttattgttgaatttttttgaattggtatagaacgttttgttattgagataaagacgttttttaatattgagttagtattaaaggatttttgttttaagagaatcgagtattgggtgattgaaaatattaaaggaattgtgtaaatatagatttttatttttttcgtcgtttttttttttcgtttttaatatattaagaaatcgaaacgtttcgttcgggagaagtagcgtagtttggattttagttttcgttggtttttttttattttgaaagagaatgggaagttttggtttttaagatttaaagatttaggggttttgcggagttcgaggggttttggcggttttaaatatttaggcgatcggagggttaagttttttatttttgttttttttttttttcggtaaattttgcgttagaggttagttttagtttcgtcgtttggtttagtatttttttttgtagttcggtttcgcgaatttttacgttcgtttgcgggttattcgggttgcgggtttattttttcgttggtgaaaaatgtatttcggtggaatgggaatatttgggaagggcgcgtataaattttatacgtttttttttacgttatttttataatatattaggattttatttatgatcgagtatattatcggatttttatttttttattattattttagttgataaagttggggtttagggtttttttcgtgtttttttttttttttcgttgggtttcggagttttttttcgttcgagaggagttcggaaggcgggaaggggaatcgttgtcgggggttagttaggtcgttatttttcgttagtcgcgggttgttcggttttttgtttttttcggcggcgtttatgtttagttttcggacgggagagcgtttttttttagttttttcgtttgttttttttttttttatatttttttcgtttttttcgtcgtcgttttcgtttttttcgtcgtcgttttcgtcggtcgttcggattgtcgggttgttgcggtcgtcgcggttcgtttcggtgtagtcggtcggttttttggtttcgcgtagcggttcgttttttagtatttttcggtacgtgatcgtttttttttttttttttattttttggttaaaagatttggagacgaacgtcgtaagtttttttatcgtcgtcgtacgttagttcggcgtttaatttttgttagtagagtttcgtttttttttttttttttttttttttattgttttagtttttttaataataatatattaatgggataggaggtgggg

SEQ ID NO: 8 - DNA, 5473 bp, Artificial Sequence
  SEQ ID NO 6 converted C to T, reverse complement
ccccacctcctatcccattaatatattattattaaaaaaactaaaacaataaaaaaaaaaaaaaaaaaaaaaaaacgaaactctactaacaaaaattaaacgccgaactaacgtacgacgacgataaaaaaacttacgacgttcgtctccaaatcttttaaccaaaaaataaaaaaaaaaaaaaaaacgatcacgtaccgaaaaatactaaaaaacgaaccgctacgcgaaaccaaaaaaccgaccgactacaccgaaacgaaccgcgacgaccgcaacaacccgacaatccgaacgaccgacgaaaacgacgacgaaaaaaacgaaaacgacgacgaaaaaaacgaaaaaaatataaaaaaaaaaaaaacaaacgaaaaaactaaaaaaaaacgctctcccgtccgaaaactaaacataaacgccgccgaaaaaaacaaaaaaccgaacaacccgcgactaacgaaaaataacgacctaactaacccccgacaacgattccccttcccgccttccgaactcctctcgaacgaaaaaaaactccgaaacccaacgaaaaaaaaaaaaaaacacgaaaaaaaccctaaaccccaactttatcaactaaaataataataaaaaaataaaaatccgataatatactcgatcataaataaaatcctaatatattataaaaataacgtaaaaaaaaacgtataaaatttatacgcgcccttcccaaatattcccattccaccgaaatacatttttcaccaacgaaaaaataaacccgcaacccgaataacccgcaaacgaacgtaaaaattcgcgaaaccgaactacaaaaaaaaatactaaaccaaacgacgaaactaaaactaacctctaacgcaaaatttaccgaaaaaaaaaaaaaacaaaaataaaaaacttaaccctccgatcgcctaaatatttaaaaccgccaaaacccctcgaactccgcaaaacccctaaatctttaaatcttaaaaaccaaaacttcccattctctttcaaaataaaaaaaaaccaacgaaaactaaaatccaaactacgctacttctcccgaacgaaacgtttcgatttcttaatatattaaaaacgaaaaaaaaaaacgacgaaaaaaataaaaatctatatttacacaattcctttaatattttcaatcacccaatactcgattctcttaaaacaaaaatcctttaatactaactcaatattaaaaaacgtctttatctcaataacaaaacgttctataccaattcaaaaaaattcaacaataaaattaatttaacacttaatacctacaataactaatatactaccatatttttccaattaaaaaaatatataacatacgtaaaatcacatatttaaacaaacaattaaattatttataacacgatctattcctatttaaatcaataattaaaaatttaaaaacaaaaataaaaattttattaaatctataaatcaacttaaaatttaaacatctcctcgaaaccccaaaactttaaataacatccactatataaataacatataataaacaaaaccttcccacctcattaacaaaatttaaaaaatatattttcatatttaaatttcaatacaatatcaataaaaatactttactattaaattaacccataatatttactataatattccttcctaaaaaaaaaacaaatatccacaaaactaactctaaaaactaaaaaatacgacaatatttaaaatcaccgtaactaaaatacccgacaaaaccgaaatctaacgactcttcttcctccaaaccaacaccaaatcttcctaaatttataaaataataaaattaccgtcaaaatacccaatacaacctcccgaccataaatccacaactacaaacccgaactacaaattcgccaacgcgcctaacgacgaaattcaaatctaaacccccgacgaacgcaattcctaaaattattaatttccaaactcaaaacaaatattaatttctctacaaaccgactcttccgaaaataatttttaacacccatactaacctaaatcctacgctaaaaaaaactacaaacaacctcgactatctaaactcaactttaacttcttaaaacccgaaatctctaattcgaacgcaaaaaataaaaattccaaaaaaatatatatataataactatatatactcacactaaaaaaactcctatttttaaaaaaaaaaaaaaaacaaaaaaataacaacgaatatatacaaacccaaaacaaaaaaattcatattttattaaacatccgaactactaattacataaaaaaactttaaacaaccaataaaaaccgaaaatcgcgaatattccgcttaaacctattaatctctataaattaaacgtttaaaatcgaatctacgtttccacgaaaaaaaacgaaaaaaaaacaaaaaaaacgattactttcgcccgccaccgaaaactaattttccgcctcctaccttctaacccgacttaaacgacgaaccctttaaacaaccaacataacgtaaacgcctatactcgtacgaccccgatcgaacaaacgaaacgaaaattacctaactatccaaaaaaccttatacaaaatttaacccgaacccaaaaacaacgaaaaacgtctacgaatacgactccctatacgacacaacaccgaaatctattactctcgctaatacctttcgacccaattattcctaaattaaaccaacaaatttacgataaacaaaaaaccaacgataataaaaactaacctaaaaacgaaccaaccttctaaaaataaccgatataaaaaaccaataaataaccttaccgcgcccttatatccaaactttactctataaaaaacctaacgctacgcctttaatttctataaatactacgaaacaaataaatacgaaaataaataaaaaccatttaatatttactaaaaaaaaccgataactaaaaaataaataataaaacgataaatcgaaatacgtttataaaaaaaataataaataactacccaccttaccccaaacgaccgctcaaacttaacctttacgcttctccgaaatattcttcctccgaataaattaaaaaaccgaaccaaaattccacgaaaaaaaaaaattctctattcttttttacgacaaatctaataacaaaatttactatactattttcgtccgtatatatatacgtatatatatatattcgtataaatacacgtataaccccgctaaaataccccctccaatatccccgaaactaaaaaatcgcaaaaaaaatacgaaaaaaataaaaaacgaaaaccaaaccaaaatcaaacaaaaacgaaatcgaaccaatttcaccctaaaacaactcaataaactaaaaaaactttttaacgaaacccactatcccgacgccttcatacgaaaaaaactaaaccaacgactaaacctatcgaaaacccgaatacaaatacccaactaccaaaaaatcgaaaacccaaaaaacaaaaaaaacctaaaattaaaaaaaaacttaaaaaacacactcgaaaaataatacttctaatatctaatataaaataaaaaaaaaaaaaatatctttactaaaaatcttattttccaaaataaatcttaatcctataacactcccctccttttttttttttccatcctaacatatattcccaaaaaaaaaaaactatactaaactttctcccaaaatttctataaaccacaccccccttacctcgcactaactttaatataatttaatcctaataaacctacttttataaaacttaaaaaaaaatcccacaaaaaacgttttatctctaaataccttaaaaattccaaaaaatattttattctctaaaattaaatttccttaaaaatctactctacaaaatctaaatttatcaaacgaatatcccaattaaatttataaatatacaaactataaaaaaaattaataactattaaatacaaaaatttacaatctactattaacgatcgatctcaaactatatataaaataacgaaaaaataaacgcattttaatcactctttattcattcatttaaccaaaacaacgaaactaaccctaaatacattaaattttaaaacaaaccacatatattaatatttatatttccaaaacctaccctccaacacgtaccatacccaataaaattaaaactaaaacaaattttctttattaattataaaaacaaatataataacaaaaataaactttaaaaacatttaaaaaaacaaattatatacattcacatacacatatccttctaactttaaaaaaaacaaatttaaatatttcaaattatatttccgctcaaataataaaataaaataaaataataattattattaatttaaacacataaaacaattttaaacccgcacatctacaactattacaaaatatcgctataacaaatactctttcgatctctaaacacaaaaaccaccatctataacgaactatacacaattcttaaaaaacttccccacatcttataacaaaatcaaaaaaaaaaaaaacaaaacaatcacgttaaaaaaaaaaaataataataaactaccttaatcatcacaacatcaacaacaaaaccttcctttttaaacctctcctacttcgctctatacactaaaatctccaaaaactcaacaaccccttaaaaacataataataattattacaattataattattaatatcttaattccaactcttctaaaaaaaaaaaaaacaaaaaacctcaaaccatataaattacacaaaaaataccaccctaacacaaaatccaccctctattatactaaataacaaaaaaatttttaaaaatttacattaactataacacttactttcaaaaacataccaataaaaactctaaaacaatatttttactttaataaaattttaaaaactttaatttataaatcaatcaatatactttattattaaaaataaaatacaaaaacaaactttatttaaattttaaaaaatctatcatactacctataaaaattatcaaatttaaacaaataacatccaaaaaaacaaaatacctaaaaattacttactttatatatacttcattctcctttcgctaaatactcttctttttttctcttaaaaaataaactttattattatttcatacttttatttacattttaattattttctaaatattttcctactatcctaactacaaacaataactttttctaaaaaaacaaattaaattatcataataataatacacaattattctactactatatttcactctactacacaacaaaacaaaaaaattctaaacttctttataataaaacttttataacactaattacctctccccaacccttacaactccttctccgcaaaaaaatcattatttactctaatatttaatttttctttaaaataaaactaattaacattac

SEQ ID NO: 9 - DNA, 5473 bp, Artificial Sequence
  SEQ ID NO 6 converted G to A
ataatattaattaacttcatcttaaaaaaaaaccaaacaccaaaacaaataataactcctttacgaaaaaaaaattataaaaattaaaaaaaaataattaatatcataaaaacctcactacaaaaaaatttaaaatttctctattctactatacaacaaaataaaacacaataacaaaataattatacaccattatcataataatctaatttacttctttaaaaaaaaccactatttacaattaaaataataaaaaaacatctaaaaaataactaaaatataaataaaaatataaaacaacaataaaattcacttcttaaaaaaaaaaaaaaaaaacactcaacgaaaaaaaaataaaatatatataaaacaaataactcttaaatactttattctcctaaatattacctacttaaacttaacaacttttacaaacaatataacaaattttttaaaattcaaataaaacttacttttatattccactttcaataataaaacatattaattaactcacaaattaaaatttttaaaaccccattaaaataaaaatactacttcaaaactttcattaatatatttttaaaaacaaatatcataatcaatataaatcttcaaaaatttctctactatttaatacaacaaaaaataaattttataccaaaataacatcctttatataattcacataacctaaaacttcttacctttttttcccctaaaaaaattaaaattaaaatactaataatcataactataataattatcatcatatttttaaaaaattactaaacctctaaaaatctcaatacacaaaacgaaacaaaaaaaacttaaaaaaaaaaactctactactaatactataataaccaaaacaacctattattacttttcctcttcaacgtaactactctatttttttttttcctaatcctattacaaaatataaaaaaaccccttaaaaattatacataattcgctacaaataataatctctatacccaaaaaccgaaaaaatatctattacaacgatactttataacaactacaaatatacgaacctaaaactactccatatattcaaaccaataataactatcattttattctattttattatctaaacgaaaatacaatctaaaacatttaaatttattccccctaaaactaaaaaaatatatatatataaatatacacaatttactcctttaaatacccccaaaacctatttttactattacatttacctccacaaccaataaaaaaaatctatttcaatcctaacttcattaaatataacacgtactaaaaaacaaactttaaaaatataaatactaatatatataatttattttaaaatctaatatatttaaaatcaatttcgttaccttaattaaataaataaacaaaaaataaccaaaatacgtttatttctccgctatttcacacacaatttaaaatcgaccgctaacaacaaactataaatttctacacttaacaattactaattttctttataatctacacatctataaacctaattaaaatatccgcttaacaaacctaaaccctacaaaacaaactttcaaaaaaatccaactccaaaaaacaaaatactctttaaaacttttaaaacatccaaaaataaaacgttttctataaaatttttttttaaaccttataaaaataaacccattaaaattaaaccacattaaaaccaatacgaaacaaaaaaaatataatttataaaaactctaaaaaaaaacccaacacaactttcttcttctaaaaacatatattaaaataaaaaaaaaaaaaaaaaaaaaaatactataaaactaaaatctactttaaaaaataaaacctctaataaaaatatctctttccttctcatcttacaccaaacactaaaaacaccacctcccgaatatatcccccaaacctctttccaaccccaaactctctctactccctaaaccctcgacctcctaacaactaaatacctacactcgaacctccgacaaacccaatcgctaactcaattcctctcgcataaaaacgtcgaaataataaatctcgtcaaaaaacctctccaactcattaaattattccaaaataaaattaatccgacttcgcctctacttaattttaatctaaccttcgtcctccatccctttcgcatcctctttacgatctttcaactccgaaaacactaaaaaaaacaccccaacgaaaccacacgtacatccacacgaacacacacacacacgcacacacacacgaacgaaaacaacacaacaaatcctattaccaaatttatcgcaaaaaaaaataaaaaatccttccccctcgtaaaatcctaatccgacttctcaacccacccgaaaaaaaaatatcccgaaaaaacgcaaaaatcaaatctaaacgaccgcctaaaacaaaataaacaatcatccaccacctccttcacaaacgtacctcgactcaccgcctcactattcaccccccaatcaccgaccttccccaacaaacaccaaataatttccactcactttcgcactcacttactccgcaacactcacaaaaatcaaaaacgcaacgccaaacctctcacaaaacaaaatctaaacataaaaacgcgacaaaaccacccactaacctctcacaccgaccattcccaaaaaactaactcgttctcaaaccaactctcaccaccgctaactctctacctaccgcaaacttactaatctaatttaaaaacaattaaaccgaaaaatatcaacgaaaacaacaaaccccgatattataccgcacaaaaaaccgcatccgcaaacgcccctcgctacccctaaactcgaaccaaaccctacataaaatcccctaaacaaccaaataatctccgtcccgcctacccgaccgaaatcgcacgaacacaaacgcccacgccatattaactacccaaaaaactcgccgcccaaaccgaaccaaaaaacaaaaaacgaaaaaccaacctccgataacgaacgaaaacaaccgctctttctattctctcttcgccctccctcgtaaaaacgcaaactcgaccctaaacgcttaacccacaaaaatcaacaaattcaaacgaaatattcgcgatcctcgatttctattaattactcaaaaccttttcatacaaccaacaactcgaatatttaataaaatataaatttttctactctaaatttacacacatccgttaccactttcctatccttctcctccctttaaaaacaaaaactcttctaatataaacatatacaaccaccatatatatatccccctaaaacctttatcctctacgcccgaatcaaaaactccgaattttaaaaaatcaaaactaaatttaaataatcgaaactatttacaattcttcccaacgcaaaatccaaaccaacataaatattaaaaatcatttccgaaaaaaccgatctacaaaaaaattaatacttaccctaaacttaaaaatcaacaatcccaaaaactacgcccgccgaaaattcaaacctaaacctcgccgtcaaacgcgctaacgaacctataattcgaacctataattataaactcataaccgaaaaactacactaaacaccttaacgataactccaccactttacaaatttaaaaaaacctaatactaatttaaaaaaaaaaaaatcgctaaatctcgaccctaccgaacattctaactacgataatcttaaatattaccgtattttctaattctcaaaattaattttataaacatttatcctttctttaaaaaaaaacattataataaacattataaaccaatttaataacaaaatatttttactaatattatattaaaatttaaatataaaaacacacccttcaaattctaccaataaaataaaaaaactttatctattacatatcatctacacaataaataccactcaaaattttaaaacttcgaaaaaatatccaaatttcaaattaatccataaacttaataaaatctctatttttatccccaaatttccaattactaatctaaacaaaaacaaaccgtatcataaacaacttaattacttacttaaatatataattccacgcataccacatattttcttaattaaaaaaatataacaatacaccaattactacaaacaccaaataccaaatcaattttattattaaactcctttaaattaatataaaacgctctaccattaaaataaaaacgtttcctaacactaaactaacaccaaaaaatttttactctaaaaaaaccgaatactaaataattaaaaacactaaaaaaattatataaatataaatccccacctcccccgccgccccccccccccgcccccaacacaccaaaaaaccgaaacgcctcgcccgaaaaaaacaacgtaacctaaacctcaactttcgttaacttccttctaccttaaaaaaaaataaaaaattttaacccctaaaatccaaaaattcaaaaactctacgaaattcgaaaaatcttaacgaccccaaacacctaaacgaccgaaaaattaaactttccacctctaccccttctccctcccgacaaactctacgctaaaaactaactttaatcccgccgtctaattcaacaccccctcctacaacccgaccccgcgaacttccacgtccgcctacgaaccatccgaactacgaacccatcctcccgctaataaaaaatacatttcgataaaataaaaacatctaaaaaaaacgcgcacaaaccccacacgcttccccccacgccacccccacaacacactaaaaccccacccataaccgaacataccaccgaacccccacccttccactatcactctaactaacaaaactaaaatccaaaaccctctccgtatccctctcccctccccgctaaacctcgaaatcctctcccgcccgaaaaaaatccgaaaaacgaaaaaaaaaaccgctaccgaaaatcaatcaaatcgttaccctccgtcaatcgcgaactacccgactccctacttctctcgacgacgcccatatccaactcccgaacgaaaaaacgccctcctccaactcctccgcctactcctcctcctcctacacctcctccgcctcctccgccgccgcctccgcctcctccgccgccgcctccgccgaccgcccgaactaccgaactactacgatcgtcgcgacccgcctcgatacaaccgatcgactccttaaccccgcgcaacgacccgctctccaacacctcccgatacgtaatcgcctccttcttctccttcactttctaatcaaaaaacttaaaaacgaacgccgtaaattcttccatcgccgccgcacgtcaacccgacgctcaacctctaccaacaaaaccccgctctttttttccttcttctttttttactactccaacccccccaataataacacatcaataaaacaaaaaataaaa

SEQ ID NO: 10 - DNA, 5473 bp, Artificial Sequence
  SEQ ID NO 6 converted G to A, reverse complement
ttttattttttgttttattgatgtgttattattgggggggttggagtagtaaaaaaagaagaaggaaaaaaagagcggggttttgttggtagaggttgagcgtcgggttgacgtgcggcggcgatggaagaatttacggcgttcgtttttaagttttttgattagaaagtgaaggagaagaaggaggcgattacgtatcgggaggtgttggagagcgggtcgttgcgcggggttaaggagtcgatcggttgtatcgaggcgggtcgcgacgatcgtagtagttcggtagttcgggcggtcggcggaggcggcggcggaggaggcggaggcggcggcggaggaggcggaggaggtgtaggaggaggaggagtaggcggaggagttggaggagggcgttttttcgttcgggagttggatatgggcgtcgtcgagagaagtagggagtcgggtagttcgcgattgacggagggtaacgatttgattgattttcggtagcggttttttttttcgtttttcggatttttttcgggcgggagaggatttcgaggtttagcggggaggggagagggatacggagagggttttggattttagttttgttagttagagtgatagtggaagggtgggggttcggtggtatgttcggttatgggtggggttttagtgtgttgtgggggtggcgtggggggaagcgtgtggggtttgtgcgcgttttttttagatgtttttattttatcgaaatgtattttttattagcgggaggatgggttcgtagttcggatggttcgtaggcggacgtggaagttcgcggggtcgggttgtaggagggggtgttgaattagacggcgggattaaagttagtttttagcgtagagtttgtcgggagggagaaggggtagaggtggaaagtttaatttttcggtcgtttaggtgtttggggtcgttaagatttttcgaatttcgtagagtttttgaatttttggattttaggggttaaaattttttatttttttttaaggtagaaggaagttaacgaaagttgaggtttaggttacgttgtttttttcgggcgaggcgtttcggttttttggtgtgttgggggcggggggggggggcggcgggggaggtggggatttatatttatataatttttttagtgtttttaattatttagtattcggtttttttagagtaaaaattttttggtgttagtttagtgttaggaaacgtttttattttaatggtagagcgttttatattaatttaaaggagtttaataataaaattgatttggtatttggtgtttgtagtaattggtgtattgttatatttttttaattaagaaaatatgtggtatgcgtggaattatatatttaagtaagtaattaagttgtttatgatacggtttgtttttgtttagattagtaattggaaatttggggataaaaatagagattttattaagtttatggattaatttgaaatttggatattttttcgaagttttaaaattttgagtggtatttattgtgtagatgatatgtaatagataaagtttttttattttattggtagaatttgaagggtgtgtttttatatttaaattttaatataatattagtaaaaatattttgttattaaattggtttataatgtttattataatgtttttttttaaagaaaggataaatgtttataaaattaattttgagaattagaaaatacggtaatatttaagattatcgtagttagaatgttcggtagggtcgagatttagcgatttttttttttttaaattagtattaggtttttttaaatttgtaaagtggtggagttatcgttaaggtgtttagtgtagtttttcggttatgagtttataattataggttcgaattataggttcgttagcgcgtttgacggcgaggtttaggtttgaattttcggcgggcgtagtttttgggattgttgatttttaagtttagggtaagtattaatttttttgtagatcggttttttcggaaatgatttttaatatttatgttggtttggattttgcgttgggaagaattgtaaatagtttcgattatttaaatttagttttgattttttaaaattcggagtttttgattcgggcgtagaggataaaggttttagggggatatatatatggtggttgtatatgtttatattagaagagtttttgtttttaaagggaggagaaggataggaaagtggtaacggatgtgtgtaaatttagagtagaaaaatttatattttattaaatattcgagttgttggttgtatgaaaaggttttgagtaattaatagaaatcgaggatcgcgaatatttcgtttgaatttgttgatttttgtgggttaagcgtttagggtcgagtttgcgtttttacgagggagggcgaagagagaatagaaagagcggttgttttcgttcgttatcggaggttggtttttcgttttttgttttttggttcggtttgggcggcgagttttttgggtagttaatatggcgtgggcgtttgtgttcgtgcgatttcggtcgggtaggcgggacggagattatttggttgtttaggggattttatgtagggtttggttcgagtttaggggtagcgaggggcgtttgcggatgcggttttttgtgcggtataatatcggggtttgttgttttcgttgatatttttcggtttaattgtttttaaattagattagtaagtttgcggtaggtagagagttagcggtggtgagagttggtttgagaacgagttagttttttgggaatggtcggtgtgagaggttagtgggtggttttgtcgcgtttttatgtttagattttgttttgtgagaggtttggcgttgcgtttttgatttttgtgagtgttgcggagtaagtgagtgcgaaagtgagtggaaattatttggtgtttgttggggaaggtcggtgattggggggtgaatagtgaggcggtgagtcgaggtacgtttgtgaaggaggtggtggatgattgtttattttgttttaggcggtcgtttagatttgatttttgcgttttttcgggatatttttttttcgggtgggttgagaagtcggattaggattttacgagggggaaggattttttatttttttttgcgataaatttggtaataggatttgttgtgttgttttcgttcgtgtgtgtgtgcgtgtgtgtgtgtgttcgtgtggatgtacgtgtggtttcgttggggtgttttttttagtgttttcggagttgaaagatcgtaaagaggatgcgaaagggatggaggacgaaggttagattaaaattaagtagaggcgaagtcggattaattttattttggaataatttaatgagttggagaggttttttgacgagatttattatttcgacgtttttatgcgagaggaattgagttagcgattgggtttgtcggaggttcgagtgtaggtatttagttgttaggaggtcgagggtttagggagtagagagagtttggggttggaaagaggtttgggggatatattcgggaggtggtgtttttagtgtttggtgtaagatgagaaggaaagagatatttttattagaggttttattttttaaagtagattttagttttatagtatttttttttttttttttttttttattttaatatatgtttttagaagaagaaagttgtgttgggtttttttttagagtttttataaattatattttttttgtttcgtattggttttaatgtggtttaattttaatgggtttatttttataaggtttaaaaaaaaattttatagaaaacgttttatttttggatgttttaaaagttttaaagagtattttgttttttggagttggatttttttgaaagtttgttttgtagggtttaggtttgttaagcggatattttaattaggtttatagatgtgtagattataaagaaaattagtaattgttaagtgtagaaatttatagtttgttgttagcggtcgattttaaattgtgtgtgaaatagcggagaaataaacgtattttggttattttttgtttatttatttaattaaggtaacgaaattgattttaaatatattagattttaaaataaattatatatattagtatttatatttttaaagtttgttttttagtacgtgttatatttaatgaagttaggattgaaatagattttttttattggttgtggaggtaaatgtaatagtaaaaataggttttgggggtatttaaaggagtaaattgtgtatatttatatatatatatttttttagttttagggggaataaatttaaatgttttagattgtattttcgtttagataataaaatagaataaaatgatagttattattggtttgaatatatggagtagttttaggttcgtatatttgtagttgttataaagtatcgttgtaatagatattttttcggtttttgggtatagagattattatttgtagcgaattatgtataatttttaaggggtttttttatattttgtaataggattaggaaaaaaaaaaatagagtagttacgttgaagaggaaaagtaataataggttgttttggttattatagtattagtagtagagtttttttttttaagttttttttgtttcgttttgtgtattgagatttttagaggtttagtaattttttaaaaatatgatgataattattatagttatgattattagtattttaattttaatttttttaggggaaaaaaaggtaagaagttttaggttatgtgaattatataaaggatgttattttggtataaaatttattttttgttgtattaaatagtagagaaatttttgaagatttatattgattatgatatttgtttttaaaaatatattaatgaaagttttgaagtagtatttttattttaatggggttttaaaaattttaatttgtgagttaattaatatgttttattattgaaagtggaatataaaagtaagttttatttgaattttaaaaaatttgttatattgtttgtaaaagttgttaagtttaagtaggtaatatttaggagaataaagtatttaagagttatttgttttatatatattttatttttttttcgttgagtgttttttttttttttttttaagaagtgaattttattgttgttttatatttttatttatattttagttattttttagatgtttttttattattttaattgtaaatagtggttttttttaaagaagtaaattagattattatgataatggtgtataattattttgttattgtgttttattttgttgtatagtagaatagagaaattttaaatttttttgtagtgaggtttttatgatattaattatttttttttaatttttataatttttttttcgtaaaggagttattatttgttttggtgtttggtttttttttaagatgaagttaattaatattat

SEQ ID NO: 11 - DNA, 1891 bp, Homo sapiens
ctcatcttacaccagacactagaagcaccacctcccgagtgtgtcccccaagcctctttccaaccccaggctctctctgctccctgggccctcgacctcctggcagctgggtacctgcactcgggcctccgacaggcccagtcgctggctcagttcctctcgcatgaaggcgtcgggatagtgggtctcgtcaaaaagcctctccagctcattgagttgttccagggtgaaattggtccgacttcgcctctgcttgattttggtctggccttcgtcctccatccctttcgcatcctctttgcgatctttcagctccggggacactggagggggcaccccagcggggccacacgtgcatccacacgaacacacacacacacgcacacacacacggacgaaaacagcacagcaaatcctgttaccagatttgtcgcaaaaaagaatagagaatccttccccctcgtggaatcctggtccggcttctcaacccacccggaggaagaatatcccggagaagcgcaaaggtcaagtctgagcggccgcctggggcaaggtgggcagtcatccaccacctccttcacaaacgtacctcgactcaccgcctcactattcaccccccagtcaccggccttccccagcaaacaccaaatggtttccactcactttcgcactcacttgctccgcagcactcacagaaatcaaaggcgcagcgccaggcctctcacagagcaaagtctggacataagggcgcggcaaggccacccactggcctctcacaccggccattcccagaaggctggctcgttctcaggccagctctcaccaccgctggctctctgcctaccgcaaacttgctggtctaatttaggaacaattgggccgaaaggtatcagcgagagcaacagaccccggtgttgtgccgcacagggagccgcatccgcagacgcccctcgctgcccctgggctcgggccaaaccctgcataaggtcccctggacagccaggtaatctccgtcccgcctgcccgaccggggtcgcacgagcacaggcgcccacgccatgttggctgcccaaagggctcgccgcccaagccgggccagaaggcaggaggcggaaaaccagcctccggtggcgggcgaaagcaaccgctctttctgttctctcttcgccctccctcgtggaaacgcagactcgaccctaaacgcttaacccacagagatcaacaggttcaagcggaatattcgcgatcctcggtttctattggttgctcaaagccttttcatgcaaccagcagctcggatgtttaataaaatatgaatttttctgctctgggtttgcacacatccgttgccactttcctgtccttctcctccctttaaaaacaggagctcttctagtgtgagcatatacagccaccatatatatatccccctggaacctttatcctctgcgcccgaatcagagactccgggttttaagaagtcaaagctgagtttagatagtcgaggctgtttgcagttcttcccagcgcaggatccaggccagcatgggtgttaaaaatcatttccggaagagccggtctgcagagaaattaatacttgccctgagcttggaaatcaacaatcccaggaactgcgcccgccgggggttcagacctgaacctcgccgtcaggcgcgctggcgaacctgtagttcgggcctgtagttgtggactcatggccgggaggctgcactgggcaccttgacggtaactccaccactttacaaatttaggaagacctggtgctggtttggaggaagaagagtcgctagatctcggccctgccgggcattctagctacggtgatcttaaatattgccgtattttctagttctcagagttagttttgtgg

SEQ ID NO: 12 - DNA, 1891 bp, Artificial Sequence
  SEQ ID NO 11 converted C to T
tttattttatattagatattagaagtattatttttcgagtgtgttttttaagtttttttttaattttaggttttttttgttttttgggttttcgattttttggtagttgggtatttgtattcgggttttcgataggtttagtcgttggtttagtttttttcgtatgaaggcgtcgggatagtgggtttcgttaaaaagtttttttagtttattgagttgttttagggtgaaattggttcgatttcgtttttgtttgattttggtttggttttcgttttttatttttttcgtattttttttgcgattttttagtttcggggatattggagggggtattttagcggggttatacgtgtatttatacgaatatatatatatacgtatatatatacggacgaaaatagtatagtaaattttgttattagatttgtcgtaaaaaagaatagagaatttttttttttcgtggaattttggttcggttttttaatttattcggaggaagaatatttcggagaagcgtaaaggttaagtttgagcggtcgtttggggtaaggtgggtagttatttattattttttttataaacgtatttcgatttatcgttttattatttattttttagttatcggttttttttagtaaatattaaatggtttttatttattttcgtatttatttgtttcgtagtatttatagaaattaaaggcgtagcgttaggttttttatagagtaaagtttggatataagggcgcggtaaggttatttattggttttttatatcggttatttttagaaggttggttcgtttttaggttagtttttattatcgttggttttttgtttatcgtaaatttgttggtttaatttaggaataattgggtcgaaaggtattagcgagagtaatagatttcggtgttgtgtcgtatagggagtcgtattcgtagacgtttttcgttgtttttgggttcgggttaaattttgtataaggttttttggatagttaggtaattttcgtttcgtttgttcgatcggggtcgtacgagtataggcgtttacgttatgttggttgtttaaagggttcgtcgtttaagtcgggttagaaggtaggaggcggaaaattagttttcggtggcgggcgaaagtaatcgttttttttgtttttttttcgttttttttcgtggaaacgtagattcgattttaaacgtttaatttatagagattaataggtttaagcggaatattcgcgattttcggtttttattggttgtttaaagtttttttatgtaattagtagttcggatgtttaataaaatatgaatttttttgttttgggtttgtatatattcgttgttatttttttgttttttttttttttttaaaaataggagtttttttagtgtgagtatatatagttattatatatatatttttttggaatttttattttttgcgttcgaattagagatttcgggttttaagaagttaaagttgagtttagatagtcgaggttgtttgtagttttttttagcgtaggatttaggttagtatgggtgttaaaaattattttcggaagagtcggtttgtagagaaattaatatttgttttgagtttggaaattaataattttaggaattgcgttcgtcgggggtttagatttgaatttcgtcgttaggcgcgttggcgaatttgtagttcgggtttgtagttgtggatttatggtcgggaggttgtattgggtattttgacggtaattttattattttataaatttaggaagatttggtgttggtttggaggaagaagagtcgttagatttcggttttgtcgggtattttagttacggtgattttaaatattgtcgtattttttagtttttagagttagttttgtgg

SEQ ID NO: 13 - DNA, 1891 bp, Artificial Sequence
  SEQ ID NO 11 converted C to T, reverse complement
ccacaaaactaactctaaaaactaaaaaatacgacaatatttaaaatcaccgtaactaaaatacccgacaaaaccgaaatctaacgactcttcttcctccaaaccaacaccaaatcttcctaaatttataaaataataaaattaccgtcaaaatacccaatacaacctcccgaccataaatccacaactacaaacccgaactacaaattcgccaacgcgcctaacgacgaaattcaaatctaaacccccgacgaacgcaattcctaaaattattaatttccaaactcaaaacaaatattaatttctctacaaaccgactcttccgaaaataatttttaacacccatactaacctaaatcctacgctaaaaaaaactacaaacaacctcgactatctaaactcaactttaacttcttaaaacccgaaatctctaattcgaacgcaaaaaataaaaattccaaaaaaatatatatataataactatatatactcacactaaaaaaactcctatttttaaaaaaaaaaaaaaaacaaaaaaataacaacgaatatatacaaacccaaaacaaaaaaattcatattttattaaacatccgaactactaattacataaaaaaactttaaacaaccaataaaaaccgaaaatcgcgaatattccgcttaaacctattaatctctataaattaaacgtttaaaatcgaatctacgtttccacgaaaaaaaacgaaaaaaaaacaaaaaaaacgattactttcgcccgccaccgaaaactaattttccgcctcctaccttctaacccgacttaaacgacgaaccctttaaacaaccaacataacgtaaacgcctatactcgtacgaccccgatcgaacaaacgaaacgaaaattacctaactatccaaaaaaccttatacaaaatttaacccgaacccaaaaacaacgaaaaacgtctacgaatacgactccctatacgacacaacaccgaaatctattactctcgctaatacctttcgacccaattattcctaaattaaaccaacaaatttacgataaacaaaaaaccaacgataataaaaactaacctaaaaacgaaccaaccttctaaaaataaccgatataaaaaaccaataaataaccttaccgcgcccttatatccaaactttactctataaaaaacctaacgctacgcctttaatttctataaatactacgaaacaaataaatacgaaaataaataaaaaccatttaatatttactaaaaaaaaccgataactaaaaaataaataataaaacgataaatcgaaatacgtttataaaaaaaataataaataactacccaccttaccccaaacgaccgctcaaacttaacctttacgcttctccgaaatattcttcctccgaataaattaaaaaaccgaaccaaaattccacgaaaaaaaaaaattctctattcttttttacgacaaatctaataacaaaatttactatactattttcgtccgtatatatatacgtatatatatatattcgtataaatacacgtataaccccgctaaaataccccctccaatatccccgaaactaaaaaatcgcaaaaaaaatacgaaaaaaataaaaaacgaaaaccaaaccaaaatcaaacaaaaacgaaatcgaaccaatttcaccctaaaacaactcaataaactaaaaaaactttttaacgaaacccactatcccgacgccttcatacgaaaaaaactaaaccaacgactaaacctatcgaaaacccgaatacaaatacccaactaccaaaaaatcgaaaacccaaaaaacaaaaaaaacctaaaattaaaaaaaaacttaaaaaacacactcgaaaaataatacttctaatatctaatataaaataaa

SEQ ID NO: 14 - DNA, 1891 bp, Artificial Sequence
  SEQ ID NO 11 converted G to A
ctcatcttacaccaaacactaaaaacaccacctcccgaatatatcccccaaacctctttccaaccccaaactctctctactccctaaaccctcgacctcctaacaactaaatacctacactcgaacctccgacaaacccaatcgctaactcaattcctctcgcataaaaacgtcgaaataataaatctcgtcaaaaaacctctccaactcattaaattattccaaaataaaattaatccgacttcgcctctacttaattttaatctaaccttcgtcctccatccctttcgcatcctctttacgatctttcaactccgaaaacactaaaaaaaacaccccaacgaaaccacacgtacatccacacgaacacacacacacacgcacacacacacgaacgaaaacaacacaacaaatcctattaccaaatttatcgcaaaaaaaaataaaaaatccttccccctcgtaaaatcctaatccgacttctcaacccacccgaaaaaaaaatatcccgaaaaaacgcaaaaatcaaatctaaacgaccgcctaaaacaaaataaacaatcatccaccacctccttcacaaacgtacctcgactcaccgcctcactattcaccccccaatcaccgaccttccccaacaaacaccaaataatttccactcactttcgcactcacttactccgcaacactcacaaaaatcaaaaacgcaacgccaaacctctcacaaaacaaaatctaaacataaaaacgcgacaaaaccacccactaacctctcacaccgaccattcccaaaaaactaactcgttctcaaaccaactctcaccaccgctaactctctacctaccgcaaacttactaatctaatttaaaaacaattaaaccgaaaaatatcaacgaaaacaacaaaccccgatattataccgcacaaaaaaccgcatccgcaaacgcccctcgctacccctaaactcgaaccaaaccctacataaaatcccctaaacaaccaaataatctccgtcccgcctacccgaccgaaatcgcacgaacacaaacgcccacgccatattaactacccaaaaaactcgccgcccaaaccgaaccaaaaaacaaaaaacgaaaaaccaacctccgataacgaacgaaaacaaccgctctttctattctctcttcgccctccctcgtaaaaacgcaaactcgaccctaaacgcttaacccacaaaaatcaacaaattcaaacgaaatattcgcgatcctcgatttctattaattactcaaaaccttttcatacaaccaacaactcgaatatttaataaaatataaatttttctactctaaatttacacacatccgttaccactttcctatccttctcctccctttaaaaacaaaaactcttctaatataaacatatacaaccaccatatatatatccccctaaaacctttatcctctacgcccgaatcaaaaactccgaattttaaaaaatcaaaactaaatttaaataatcgaaactatttacaattcttcccaacgcaaaatccaaaccaacataaatattaaaaatcatttccgaaaaaaccgatctacaaaaaaattaatacttaccctaaacttaaaaatcaacaatcccaaaaactacgcccgccgaaaattcaaacctaaacctcgccgtcaaacgcgctaacgaacctataattcgaacctataattataaactcataaccgaaaaactacactaaacaccttaacgataactccaccactttacaaatttaaaaaaacctaatactaatttaaaaaaaaaaaaatcgctaaatctcgaccctaccgaacattctaactacgataatcttaaatattaccgtattttctaattctcaaaattaattttataa

SEQ ID NO: 15 - DNA, 1891 bp, Artificial Sequence
  SEQ ID NO 11 converted G to A, reverse complement
ttataaaattaattttgagaattagaaaatacggtaatatttaagattatcgtagttagaatgttcggtagggtcgagatttagcgatttttttttttttaaattagtattaggtttttttaaatttgtaaagtggtggagttatcgttaaggtgtttagtgtagtttttcggttatgagtttataattataggttcgaattataggttcgttagcgcgtttgacggcgaggtttaggtttgaattttcggcgggcgtagtttttgggattgttgatttttaagtttagggtaagtattaatttttttgtagatcggttttttcggaaatgatttttaatatttatgttggtttggattttgcgttgggaagaattgtaaatagtttcgattatttaaatttagttttgattttttaaaattcggagtttttgattcgggcgtagaggataaaggttttagggggatatatatatggtggttgtatatgtttatattagaagagtttttgtttttaaagggaggagaaggataggaaagtggtaacggatgtgtgtaaatttagagtagaaaaatttatattttattaaatattcgagttgttggttgtatgaaaaggttttgagtaattaatagaaatcgaggatcgcgaatatttcgtttgaatttgttgatttttgtgggttaagcgtttagggtcgagtttgcgtttttacgagggagggcgaagagagaatagaaagagcggttgttttcgttcgttatcggaggttggtttttcgttttttgttttttggttcggtttgggcggcgagttttttgggtagttaatatggcgtgggcgtttgtgttcgtgcgatttcggtcgggtaggcgggacggagattatttggttgtttaggggattttatgtagggtttggttcgagtttaggggtagcgaggggcgtttgcggatgcggttttttgtgcggtataatatcggggtttgttgttttcgttgatatttttcggtttaattgtttttaaattagattagtaagtttgcggtaggtagagagttagcggtggtgagagttggtttgagaacgagttagttttttgggaatggtcggtgtgagaggttagtgggtggttttgtcgcgtttttatgtttagattttgttttgtgagaggtttggcgttgcgtttttgatttttgtgagtgttgcggagtaagtgagtgcgaaagtgagtggaaattatttggtgtttgttggggaaggtcggtgattggggggtgaatagtgaggcggtgagtcgaggtacgtttgtgaaggaggtggtggatgattgtttattttgttttaggcggtcgtttagatttgatttttgcgttttttcgggatatttttttttcgggtgggttgagaagtcggattaggattttacgagggggaaggattttttatttttttttgcgataaatttggtaataggatttgttgtgttgttttcgttcgtgtgtgtgtgcgtgtgtgtgtgtgttcgtgtggatgtacgtgtggtttcgttggggtgttttttttagtgttttcggagttgaaagatcgtaaagaggatgcgaaagggatggaggacgaaggttagattaaaattaagtagaggcgaagtcggattaattttattttggaataatttaatgagttggagaggttttttgacgagatttattatttcgacgtttttatgcgagaggaattgagttagcgattgggtttgtcggaggttcgagtgtaggtatttagttgttaggaggtcgagggtttagggagtagagagagtttggggttggaaagaggtttgggggatatattcgggaggtggtgtttttagtgtttggtgtaagatgag

SEQ ID NO: 16 - DNA, 1008 bp, Homo sapiens
cactattcaccccccagtcaccggccttccccagcaaacaccaaatggtttccactcactttcgcactcacttgctccgcagcactcacagaaatcaaaggcgcagcgccaggcctctcacagagcaaagtctggacataagggcgcggcaaggccacccactggcctctcacaccggccattcccagaaggctggctcgttctcaggccagctctcaccaccgctggctctctgcctaccgcaaacttgctggtctaatttaggaacaattgggccgaaaggtatcagcgagagcaacagaccccggtgttgtgccgcacagggagccgcatccgcagacgcccctcgctgcccctgggctcgggccaaaccctgcataaggtcccctggacagccaggtaatctccgtcccgcctgcccgaccggggtcgcacgagcacaggcgcccacgccatgttggctgcccaaagggctcgccgcccaagccgggccagaaggcaggaggcggaaaaccagcctccggtggcgggcgaaagcaaccgctctttctgttctctcttcgccctccctcgtggaaacgcagactcgaccctaaacgcttaacccacagagatcaacaggttcaagcggaatattcgcgatcctcggtttctattggttgctcaaagccttttcatgcaaccagcagctcggatgtttaataaaatatgaatttttctgctctgggtttgcacacatccgttgccactttcctgtccttctcctccctttaaaaacaggagctcttctagtgtgagcatatacagccaccatatatatatccccctggaacctttatcctctgcgcccgaatcagagactccgggttttaagaagtcaaagctgagtttagatagtcgaggctgtttgcagttcttcccagcgcaggatccaggccagcatgggtgttaaaaatcatttccggaagagccggtctgcagagaaattaatacttgccctgagcttgg

SEQ ID NO: 17 - DNA, 1008 bp, Artificial Sequence
  SEQ ID NO 16 converted C to T
tattatttattttttagttatcggttttttttagtaaatattaaatggtttttatttattttcgtatttatttgtttcgtagtatttatagaaattaaaggcgtagcgttaggttttttatagagtaaagtttggatataagggcgcggtaaggttatttattggttttttatatcggttatttttagaaggttggttcgtttttaggttagtttttattatcgttggttttttgtttatcgtaaatttgttggtttaatttaggaataattgggtcgaaaggtattagcgagagtaatagatttcggtgttgtgtcgtatagggagtcgtattcgtagacgtttttcgttgtttttgggttcgggttaaattttgtataaggttttttggatagttaggtaattttcgtttcgtttgttcgatcggggtcgtacgagtataggcgtttacgttatgttggttgtttaaagggttcgtcgtttaagtcgggttagaaggtaggaggcggaaaattagttttcggtggcgggcgaaagtaatcgttttttttgtttttttttcgttttttttcgtggaaacgtagattcgattttaaacgtttaatttatagagattaataggtttaagcggaatattcgcgattttcggtttttattggttgtttaaagtttttttatgtaattagtagttcggatgtttaataaaatatgaatttttttgttttgggtttgtatatattcgttgttatttttttgttttttttttttttttaaaaataggagtttttttagtgtgagtatatatagttattatatatatatttttttggaatttttattttttgcgttcgaattagagatttcgggttttaagaagttaaagttgagtttagatagtcgaggttgtttgtagttttttttagcgtaggatttaggttagtatgggtgttaaaaattattttcggaagagtcggtttgtagagaaattaatatttgttttgagtttgg

SEQ ID NO: 18 - DNA, 1008 bp, Artificial Sequence
  SEQ ID NO 16 converted C to T, reverse complement
ccaaactcaaaacaaatattaatttctctacaaaccgactcttccgaaaataatttttaacacccatactaacctaaatcctacgctaaaaaaaactacaaacaacctcgactatctaaactcaactttaacttcttaaaacccgaaatctctaattcgaacgcaaaaaataaaaattccaaaaaaatatatatataataactatatatactcacactaaaaaaactcctatttttaaaaaaaaaaaaaaaacaaaaaaataacaacgaatatatacaaacccaaaacaaaaaaattcatattttattaaacatccgaactactaattacataaaaaaactttaaacaaccaataaaaaccgaaaatcgcgaatattccgcttaaacctattaatctctataaattaaacgtttaaaatcgaatctacgtttccacgaaaaaaaacgaaaaaaaaacaaaaaaaacgattactttcgcccgccaccgaaaactaattttccgcctcctaccttctaacccgacttaaacgacgaaccctttaaacaaccaacataacgtaaacgcctatactcgtacgaccccgatcgaacaaacgaaacgaaaattacctaactatccaaaaaaccttatacaaaatttaacccgaacccaaaaacaacgaaaaacgtctacgaatacgactccctatacgacacaacaccgaaatctattactctcgctaatacctttcgacccaattattcctaaattaaaccaacaaatttacgataaacaaaaaaccaacgataataaaaactaacctaaaaacgaaccaaccttctaaaaataaccgatataaaaaaccaataaataaccttaccgcgcccttatatccaaactttactctataaaaaacctaacgctacgcctttaatttctataaatactacgaaacaaataaatacgaaaataaataaaaaccatttaatatttactaaaaaaaaccgataactaaaaaataaataata

SEQ ID NO: 19 - DNA, 1008 bp, Artificial Sequence
  SEQ ID NO 16 converted G to A
cactattcaccccccaatcaccgaccttccccaacaaacaccaaataatttccactcactttcgcactcacttactccgcaacactcacaaaaatcaaaaacgcaacgccaaacctctcacaaaacaaaatctaaacataaaaacgcgacaaaaccacccactaacctctcacaccgaccattcccaaaaaactaactcgttctcaaaccaactctcaccaccgctaactctctacctaccgcaaacttactaatctaatttaaaaacaattaaaccgaaaaatatcaacgaaaacaacaaaccccgatattataccgcacaaaaaaccgcatccgcaaacgcccctcgctacccctaaactcgaaccaaaccctacataaaatcccctaaacaaccaaataatctccgtcccgcctacccgaccgaaatcgcacgaacacaaacgcccacgccatattaactacccaaaaaactcgccgcccaaaccgaaccaaaaaacaaaaaacgaaaaaccaacctccgataacgaacgaaaacaaccgctctttctattctctcttcgccctccctcgtaaaaacgcaaactcgaccctaaacgcttaacccacaaaaatcaacaaattcaaacgaaatattcgcgatcctcgatttctattaattactcaaaaccttttcatacaaccaacaactcgaatatttaataaaatataaatttttctactctaaatttacacacatccgttaccactttcctatccttctcctccctttaaaaacaaaaactcttctaatataaacatatacaaccaccatatatatatccccctaaaacctttatcctctacgcccgaatcaaaaactccgaattttaaaaaatcaaaactaaatttaaataatcgaaactatttacaattcttcccaacgcaaaatccaaaccaacataaatattaaaaatcatttccgaaaaaaccgatctacaaaaaaattaatacttaccctaaacttaa

SEQ ID NO: 20 - DNA, 1008 bp, Artificial Sequence
  SEQ ID NO 16 converted G to A, reverse complement
ttaagtttagggtaagtattaatttttttgtagatcggttttttcggaaatgatttttaatatttatgttggtttggattttgcgttgggaagaattgtaaatagtttcgattatttaaatttagttttgattttttaaaattcggagtttttgattcgggcgtagaggataaaggttttagggggatatatatatggtggttgtatatgtttatattagaagagtttttgtttttaaagggaggagaaggataggaaagtggtaacggatgtgtgtaaatttagagtagaaaaatttatattttattaaatattcgagttgttggttgtatgaaaaggttttgagtaattaatagaaatcgaggatcgcgaatatttcgtttgaatttgttgatttttgtgggttaagcgtttagggtcgagtttgcgtttttacgagggagggcgaagagagaatagaaagagcggttgttttcgttcgttatcggaggttggtttttcgttttttgttttttggttcggtttgggcggcgagttttttgggtagttaatatggcgtgggcgtttgtgttcgtgcgatttcggtcgggtaggcgggacggagattatttggttgtttaggggattttatgtagggtttggttcgagtttaggggtagcgaggggcgtttgcggatgcggttttttgtgcggtataatatcggggtttgttgttttcgttgatatttttcggtttaattgtttttaaattagattagtaagtttgcggtaggtagagagttagcggtggtgagagttggtttgagaacgagttagttttttgggaatggtcggtgtgagaggttagtgggtggttttgtcgcgtttttatgtttagattttgttttgtgagaggtttggcgttgcgtttttgatttttgtgagtgttgcggagtaagtgagtgcgaaagtgagtggaaattatttggtgtttgttggggaaggtcggtgattggggggtgaatagtg

SEQ ID NO: 21 - DNA, 112 bp, Homo sapiens
gtcccctggacagccaggtaatctccgtcccgcctgcccgaccggggtcgcacgagcacaggcgcccacgccatgttggctgcccaaagggctcgccgcccaagccgggcca

SEQ ID NO: 22 - DNA, 112 bp, Artificial Sequence
  SEQ ID NO 21 converted C to T
gttttttggatagttaggtaattttcgtttcgtttgttcgatcggggtcgtacgagtataggcgtttacgttatgttggttgtttaaagggttcgtcgtttaagtcgggtta

SEQ ID NO: 23 - DNA, 112 bp, Artificial Sequence
  SEQ ID NO 21 converted C to T, reverse complement
taacccgacttaaacgacgaaccctttaaacaaccaacataacgtaaacgcctatactcgtacgaccccgatcgaacaaacgaaacgaaaattacctaactatccaaaaaac

SEQ ID NO: 24 - DNA, 112 bp, Artificial Sequence
  SEQ ID NO 21 converted G to A
atcccctaaacaaccaaataatctccgtcccgcctacccgaccgaaatcgcacgaacacaaacgcccacgccatattaactacccaaaaaactcgccgcccaaaccgaacca

SEQ ID NO: 25 - DNA, 112 bp, Artificial Sequence
  SEQ ID NO 21 converted G to A, reverse complement
tggttcggtttgggcggcgagttttttgggtagttaatatggcgtgggcgtttgtgttcgtgcgatttcggtcgggtaggcgggacggagattatttggttgtttaggggat

SEQ ID NO: 26 - DNA, 124 bp, Homo sapiens
gtcccctggacagccaggtaatctccgtcccgcctgcccgaccggggtcgcacgagcacaggcgcccacgccatgttggctgcccaaagggctcgccgcccaagccgggccagaaggcaggagg

SEQ ID NO: 27 - DNA, 124 bp, Artificial Sequence
  SEQ ID NO 26 converted C to T
gttttttggatagttaggtaattttcgtttcgtttgttcgatcggggtcgtacgagtataggcgtttacgttatgttggttgtttaaagggttcgtcgtttaagtcgggttagaaggtaggagg

SEQ ID NO: 28 - DNA, 124 bp, Artificial Sequence
  SEQ ID NO 26 converted C to T, reverse complement
cctcctaccttctaacccgacttaaacgacgaaccctttaaacaaccaacataacgtaaacgcctatactcgtacgaccccgatcgaacaaacgaaacgaaaattacctaactatccaaaaaac

SEQ ID NO: 29 - DNA, 124 bp, Artificial Sequence
  SEQ ID NO 26 converted G to A
atcccctaaacaaccaaataatctccgtcccgcctacccgaccgaaatcgcacgaacacaaacgcccacgccatattaactacccaaaaaactcgccgcccaaaccgaaccaaaaaacaaaaaa

SEQ ID NO: 30 - DNA, 120 bp, Artificial Sequence
  SEQ ID NO 26 converted G to A, reverse complement
ttttttgttttttggttcggtttgggcggcgagttttttgggtagttaatatggcgtgggcgtttgtgttcgtgcgatttcggtcgggtaggcgggacggagattatttggttgtttagg

SEQ ID NO: 31 - DNA, 22 bp, Artificial Sequence
  shox2 forward primer
gttttttggatagttaggtaat

SEQ ID NO: 32 - DNA, 37 bp, Artificial Sequence
  shox2 forward blocker
taatttttgttttgtttgtttgattggggttgtatga

SEQ ID NO: 33 - DNA, 20 bp, Artificial Sequence
  shox2 reverse primer
taacccgacttaaacgacga

SEQ ID NO: 34 - DNA, 18 bp, Artificial Sequence
  shox2 probe
ctcgtacgaccccgatcg

SEQ ID NO: 35 - DNA, 18 bp, Artificial Sequence
  shox2 reverse primer A
cctcctaccttctaaccc

SEQ ID NO: 36 - DNA, 26 bp, Artificial Sequence
  shox2 reverse blocker
acccaacttaaacaacaaacccttta